SEQUENCE LISTING (53 sequences)

SEQ ID NO: 1 - protein, 9 aa, synthetic construct
  SITE 8: note = Alanine or Isoleucine or Leucine or Phenylalanine or Valine or Proline or Glycine
VMAPRTLXL

SEQ ID NO: 2 - protein, 9 aa, synthetic construct
VMAPRTLFL

SEQ ID NO: 3 - protein, 9 aa, synthetic construct
VMAPRTLAL

SEQ ID NO: 4 - protein, 9 aa, synthetic construct
VMAPRTLIL

SEQ ID NO: 5 - protein, 9 aa, synthetic construct
VMAPRTLLL

SEQ ID NO: 6 - protein, 9 aa, synthetic construct
VMAPRTLVL

SEQ ID NO: 7 - protein, 9 aa, synthetic construct
VMAPRTLPL

SEQ ID NO: 8 - protein, 9 aa, synthetic construct
VMAPRTLGL

SEQ ID NO: 9 - protein, 9 aa, synthetic construct
VMAPRTVLL

SEQ ID NO: 10 - protein, 9 aa, synthetic construct
VMAPRALLL

SEQ ID NO: 11 - DNA, 27 bp, synthetic construct
  source 1..27: mol_type = other DNA
gtgatggcgccgcgcaccctgtttctg

SEQ ID NO: 12 - DNA, 30 bp, synthetic construct
  source 1..30: mol_type = other DNA
gtgatggcccctagaaccctgttcctgtga

SEQ ID NO: 13 - DNA, 27 bp, synthetic construct
  source 1..27: mol_type = other DNA
gtnatggcnccnmgnacnytnttyytn

SEQ ID NO: 14 - DNA, 27 bp, synthetic construct
  source 1..27: mol_type = other DNA
gtgatggcgccgcgcaccctggcgctg

SEQ ID NO: 15 - DNA, 27 bp, synthetic construct
  source 1..27: mol_type = other DNA
gtnatggcnccnmgnacnytngcnytn

SEQ ID NO: 16 - DNA, 27 bp, synthetic construct
  source 1..27: mol_type = other DNA
gtgatggcgccgcgcaccctgattctg

SEQ ID NO: 17 - DNA, 27 bp, synthetic construct
  source 1..27: mol_type = other DNA
gtnatggcnccnmgnacnytnathytn

SEQ ID NO: 18 - DNA, 27 bp, synthetic construct
  source 1..27: mol_type = other DNA
gtgatggcgccgcgcaccctgctgctg

SEQ ID NO: 19 - DNA, 27 bp, synthetic construct
  source 1..27: mol_type = other DNA
gtnatggcnccnmgnacnytnytnytn

SEQ ID NO: 20 - DNA, 27 bp, synthetic construct
  source 1..27: mol_type = other DNA
gtgatggcgccgcgcaccctggtgctg

SEQ ID NO: 21 - DNA, 27 bp, synthetic construct
  source 1..27: mol_type = other DNA
gtnatggcnccnmgnacnytngtnytn

SEQ ID NO: 22 - DNA, 27 bp, synthetic construct
  source 1..27: mol_type = other DNA
gtgatggcgccgcgcaccctgccgctg

SEQ ID NO: 23 - DNA, 27 bp, synthetic construct
  source 1..27: mol_type = other DNA
gtnatggcnccnmgnacnytnccnytn

SEQ ID NO: 24 - DNA, 27 bp, synthetic construct
  source 1..27: mol_type = other DNA
gtgatggcgccgcgcaccctgggcctg

SEQ ID NO: 25 - DNA, 27 bp, synthetic construct
  source 1..27: mol_type = other DNA
gtnatggcnccnmgnacnytnggnytn

SEQ ID NO: 26 - DNA, 596 bp, synthetic construct
  source 1..596: mol_type = other DNA
gccaccatgcctaatcatcagtccgggtcacctaccggcagttcagacctgctccttgatggcaagaaacaacgagcccatctggcgctgaggagaaaacggcgacgggaaatgcgcaagattaaccgaaaggtgagaagaatgaatctcgcacccattaaagaaaaaacagcctggcagcacctgcaagctctgatcttcgaggcggaagaagtgttgaagacttctcaaactccacagacctccctcacgctgtttctagcactcttggccgtattggccccgccacctgtgagcggggtgatggcccctagaaccctgttcctgtgaagctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgc

SEQ ID NO: 27 - protein, 9 aa, synthetic construct
  VARIANT 1: note = Threonine or Valine
  VARIANT 2: note = Glycine or Asparagine
  VARIANT 4: note = Tryptophan or Glycine
  VARIANT 8: note = Tryptophan or Phenylalanine
XXPXRSLXJ

SEQ ID NO: 28 - protein, 9 aa, synthetic construct
  VARIANT 1: note = Threonine or Valine
  VARIANT 2: note = Alanine or Asparagine
  VARIANT 4: note = Alanine or Glycine
  VARIANT 6: note = Serine or Threonine
  VARIANT 7: note = Leucine or Methionine
XXPXRXXFL

SEQ ID NO: 29 - skipped

SEQ ID NO: 30 - protein, 9 aa, synthetic construct
QMPSRSLLF

SEQ ID NO: 31 - protein, 9 aa, synthetic construct
TLPKRGLFL

SEQ ID NO: 32 - protein, 9 aa, synthetic construct
TGPWRSLWI

SEQ ID NO: 33 - protein, 9 aa, synthetic construct
ILTDRSLWL

SEQ ID NO: 34 - protein, 9 aa, synthetic construct
VNPGRSLFL

SEQ ID NO: 35 - protein, 9 aa, synthetic construct
WNRLFPPLR

SEQ ID NO: 36 - protein, 9 aa, synthetic construct
TLPERTLYL

SEQ ID NO: 37 - protein, 9 aa, synthetic construct
FLPNRSLLF

SEQ ID NO: 38 - protein, 9 aa, synthetic construct
VMGDRSVLY

SEQ ID NO: 39 - protein, 9 aa, synthetic construct
VMADKSIFY

SEQ ID NO: 40 - protein, 9 aa, synthetic construct
VMPPRTLLL

SEQ ID NO: 41 - protein, 9 aa, synthetic construct
VMPGRTLCF

SEQ ID NO: 42 - protein, 9 aa, synthetic construct
RMPPRSVLL

SEQ ID NO: 43 - protein, 9 aa, synthetic construct
TAPARTMFL

SEQ ID NO: 44 - protein, 9 aa, synthetic construct
NMPARTVLF

SEQ ID NO: 45 - protein, 9 aa, synthetic construct
VLPHRTQFL

SEQ ID NO: 46 - protein, 8 aa, synthetic construct
LTDRSLWL

SEQ ID NO: 47 - DNA, 294 bp, synthetic construct
  source 1..294: mol_type = other DNA
atgcctaatcatcagtccgggtcacctaccggcagttcagacctgctccttgatggcaagaaacaacgagcccatctggcgctgaggagaaaacggcgacgggaaatgcgcaagattaaccgaaaggtgagaagaatgaatctcgcacccattaaagaaaaaacagcctggcagcacctgcaagctctgatcttcgaggcggaagaagtgttgaagacttctcaaactccacagacctccctcacgctgtttctagcactcttggccgtattggccccgccacctgtgagcggg

SEQ ID NO: 48 - protein, 98 aa, synthetic construct
MPNHQSGSPTGSSDLLLDGKKQRAHLALRRKRRREMRKINRKVRRMNLAPIKEKTAWQHLQALIFEAEEVLKTSQTPQTSLTLFLALLAVLAPPPVSG

SEQ ID NO: 49 - DNA, 266 bp, synthetic construct
  source 1..266: mol_type = other DNA
agctcgctttcttgctgtccaatttctattaaaggttcctttgttccctaagtccaactactaaactgggggatattatgaagggccttgagcatctggattctgcctaataaaaaacatttattttcattgcagctcgctttcttgctgtccaatttctattaaaggttcctttgttccctaagtccaactactaaactgggggatattatgaagggccttgagcatctggattctgcctaataaaaaacatttattttcattgc

SEQ ID NO: 50 - DNA, 64 bp, synthetic construct
  source 1..64: mol_type = other DNA
ttaaaggttcctttgttccctaagtccaactactaaactgggggatattatgaagggccttgag

SEQ ID NO: 51 - DNA, 14 bp, synthetic construct
  source 1..14: mol_type = other DNA
ttcctttgttccct

SEQ ID NO: 52 - DNA, 12 bp, synthetic construct
  source 1..12: mol_type = other DNA
gggggatattat

SEQ ID NO: 53 - protein, 9 aa, synthetic construct
VMAPQSLLL

(19) United States
(12) Patent Application Publication
(10) Pub. No.: US 2026/0286356 A1
(43) Pub. Date: Sep. 24, 2026

(54) NUCLEIC ACID VACCINE FOR ACTIVATING NKG2C+ NATURAL KILLER CELLS

(71) Applicants: Charité-Universitätsmedizin Berlin, Berlin (DE); Deutsches Rheuma-Forschungszentrum Berlin, Berlin (DE)

(72) Inventors: Chiara Romagnani, Berlin (DE); Timo Rückert, Berlin (DE)

(21) Appl. No.: 19/476,061

(22) PCT Filed: Apr. 17, 2024
(86) PCT No.: PCT/EP2024/060423

§ 371 Date: Oct. 16, 2025

(30) Foreign Application Priority Data
Apr. 18, 2023 (EP) 23168473.9

(51) Int. Cl. C12N 15/113; A61K 31/7105; A61K 38/00; A61K 39/00; A61K 48/00; A61P 35/00; C07K 14/005

(52) U.S. Cl. CPC C12N 15/113; A61K 31/7105; A61K 38/00; A61K 39/00; A61K 48/005; A61P 35/00; C07K 14/005; A61K 2039/53; A61K 2039/585; C07K 2319/04; C12N 2310/317; C12N 2800/80; C12N 2830/50

(57) ABSTRACT

A nucleic acid molecule including: (a) a sequence encoding a human cytomegalovirus (HCMV) UL40 signal peptide, or sequence variant thereof, and (b) an endoplasmic reticulum (ER)-targeting sequence. In a preferred embodiment the nucleic acid molecule further includes a stability inducing motif at its 3′-end. Also disclosed is a pharmaceutical composition including the nucleic acid molecule and a method of treating and/or reducing the risk of a medical condition associated with pathogenic cells expressing HLA-E, including administering a therapeutically effective amount of the nucleic acid molecule to a patient.

The invention is in the fields of biotechnology, molecular therapeutics and medical treatment using nucleic acids.

The invention relates to a nucleic acid molecule, comprising (a) a sequence encoding a human cytomegalovirus (HCMV) UL40 signal peptide, or sequence variant thereof, and (b) an endoplasmic reticulum (ER)-targeting sequence. In a preferred embodiment the nucleic acid molecule further comprises a stability inducing motif at its 3′-end.

The invention therefore relates to a nucleic acid molecule, comprising (a) a nucleic acid sequence encoding a peptide sequence X₁₀X₁₁X₁₂X₁₃RX₁₄X₁₅X₁₆X₁₇ (SEQ ID NO: 29), or a sequence at least 70%, 80%, 90% or 95% identical thereto, wherein X₁₀ is Threonine, Isoleucine, Phenylalanine, Asparagine, Arginine, Glutamine or Valine, X₁₁ is Glycine, Asparagine, Alanine, Leucine or Methionine, X₁₂ is Threonine or Proline, X₁₃ is Alanine, Tryptophan, Glycine, Proline, Histidine, Asparagine, Aspartic acid, Glutamic acid, Lysine or Serine, R is Arginine, X₁₄ is Serine, Glycine or Threonine, X₁₅ is Leucine, Valine, Glutamine or Methionine, X₁₆ is Tryptophan, Tyrosine, Alanine, Isoleucine, Leucine, Phenylalanine, Valine, Proline, Cysteine or Glycine, and X₁₇ is Leucine, Phenylalanine or Isoleucine, and (b) an endoplasmic reticulum (ER)-targeting sequence. In a preferred embodiment the nucleic acid molecule further comprises a stability inducing motif at its 3′-end.

The invention further relates to a nucleic acid molecule, comprising (a) a nucleic acid sequence encoding the peptide sequence VMAPRTLXL (SEQ ID NO: 1), wherein X is an amino acid with a hydrophobic side chain selected from A, I, L, F, V, P and G, and (b) an endoplasmic reticulum (ER)-targeting sequence, preferably with (c) a stability inducing motif at its 3′-end. In another aspect the invention relates to the nucleic acid molecule according to the invention for use as a medicament or as an immunogenic composition. In a further aspect the invention relates to a pharmaceutical composition comprising the nucleic acid molecule according to the invention.

### BACKGROUND OF THE INVENTION

Human cytomegalovirus (HCMV) is widespread in the general population, with the age-adjusted prevalence in Germany being around 30%. While HCMV poses only a relatively mild threat for healthy adult individuals, it is a major cause of morbidity and mortality in immunocompromised individuals and during pregnancy. Especially patients undergoing hematopoietic stem cell transplantation (HSCT) are at high risk of reactivating the virus with potentially lethal consequences. Congenital infection is associated with microcephaly, mental disabilities, and hearing problems. About 1 in 100 to 500 babies is born with congenital HCMV, and of the 10-20% symptomatic infections, 30% are lethal, making this a large-scale global health problem. In addition, a growing body of evidence suggests that the infection is associated with atherosclerosis, malignancies, autoimmune diseases, or reduced responsiveness to vaccination against influenza. Accordingly, significant efforts have been invested into developing a vaccine against HCMV, although so far none of these approaches has been successful.

While large-scale efforts in prevention and significant improvements in treatment strategies have been successful recently in reducing both cancer incidence and mortality rates, especially advanced tumors still remain a challenge in modern medicine. Increasing lifespan in industrialized and developing countries means that absolute incidence and mortality numbers are on the rise, which opens opportunities for developing more specific therapeutic approaches to treat subtypes of cancer. One particularly successful concept which recently has found its way into the clinic with impressive results is cancer immunotherapy, with checkpoint inhibition having shown success. Apart from therapies that target T cells as immune effectors, a multitude of therapeutics enhancing NK cell function, e.g., by engaging/blocking antibodies, but also as cellular therapies, is currently in clinical development. In this context, the importance of the HLA-E/NKG2 axis is well documented. Many tumor types express high levels of the non-classical MHC molecule HLA-E, and therapies targeting the HLA-E-binding inhibitory receptor NKG2A are under development¹, with several clinical trials testing the efficacy of the NKG2A blocking antibody Monalizumab for anti-tumor therapy.

Natural killer (NK) cells are cytotoxic innate immune cells, which contribute to early immune responses against viral infections⁴⁷. Their role in host protection is highlighted by patients with primary NK-cell deficiencies, who suffer from severe and disseminated viral infections caused by herpesviruses such as human cytomegalovirus (HCMV)⁴⁸; and further supported by studies of the murine CMV (MCMV) infection model⁴⁹. HCMV has a high prevalence in the adult human population and establishes life-long latency in healthy individuals. The host innate and adaptive immune systems jointly play a crucial role in restraining viral replication and preventing disease but do not eliminate the virus, which in turn engages in a dynamic interaction with the host, resulting in drastically imprinted immune-cell repertoires⁵⁰.

The anti-viral and anti-tumor effects of NK cells are well described in the scientific literature. Specifically, NK cells are protective against CMV infection in mouse models^{2,3} and have been implicated especially in the control of herpesvirus infections in humans⁴. In the context of murine and human CMV, subsets of NK cells expand specifically, accompanied by epigenetic imprinting that endows these memory NK cells with enhanced effector functions⁵-10. In humans, HCMV-induced memory NK cells express the activating receptor NKG2C, which recognizes the non-classical MHC class I molecule HLA-E loaded with endogenous peptides or peptides derived from the HCMV UL40 protein¹¹⁻¹³.

The non-classical MHC class I molecule HLA-E serves as cognate ligand for NKG2C as well as its inhibitory counterpart CD94/NKG2A (NKG2A^{51, 52, 53}) and has been reported to elicit effector functions in adaptive NKG2C+ NK cells⁴⁶ as well as to contribute to their expansion in vitro^{38,56}. Cell surface stabilization of HLA-E requires loading with peptides, which can be derived from the signal sequences of MHC class I molecules⁵⁷ or other proteins such as HSP60⁵⁸ at steady state. In addition to host peptides, the UL40 gene of HCMV was found to encode HLA-E-stabilizing peptides, which partially mimic MHC class I signal sequences^{59, 60, 61, 62}. Despite HCMV-mediated down-regulation of HLA class I to evade recognition by CD8+ T cells, UL40-derived peptides permit maintenance of HLA-E surface expression on infected cells and thereby preserve inhibition of NK-cell activation via engagement of NKG2A. Indeed, it was demonstrated that co-transfection of UL40 and HLA-E confers protection against NKG2A+ NK-cell lines and infection of fibroblasts with UL40-competent HCMV inhibits cytotoxic activity of NKG2A+ NK cells^{59, 60, 61, 62}.

Therapeutic approaches have been proposed that seek to expand and/or activate NKG2C+ natural killer (NK) cells, as disclosed for example in EP3539553B1 or U.S. Pat. No. 10,864,245B2. Such approaches are based on the therapeutic application of cytomegalovirus (HCMV) UL40 signal peptides, or sequence variants thereof, and are shown to be useful in models for the treatment and/or prevention of a medical condition associated with pathogenic cells expressing HLA-E, such as HCMV infection or cancer. Despite these approaches showing significant promise, the administration of peptide reagents is challenging due to potential issues with poor immunogenicity without adjuvants, low levels of antigen-uptake and potentially low peptide stability.

In light of the prior art, there remains a significant need to provide means or agents for activating and/or expanding therapeutically beneficial NKG2C+ NK cells and/or T cells, and for developing more effective solutions in delivering such agents in medical treatments.

### SUMMARY OF THE INVENTION

In light of the prior art, the technical problem underlying the present invention is to provide alternative and/or improved means for expanding and/or activating NKG2C+ natural killer (NK) cells and/or T cells. A further problem to be solved is the provision of means for the treatment and/or prevention of a medical condition associated with pathogenic cells expressing HLA-E and a peptide comprising an amino acid sequence according to SEQ ID NO 1 (e.g., VMAPRTLXL), such as a HCMV infection or cancer. A further problem underlying the invention is the provision of improved or alternative means for delivering cytomegalovirus (HCMV) UL40 signal peptides, or sequence variants thereof, in medical treatments.

The technical problem underlying the present invention is solved by the features of the independent claims. Preferred embodiments of the present invention are provided by the dependent claims.

The invention therefore relates to a nucleic acid molecule, comprising (a) a sequence encoding a human cytomegalovirus (HCMV) UL40 signal peptide, or sequence variants thereof, and (b) an endoplasmic reticulum (ER)-targeting sequence. In a preferred embodiment the nucleic acid molecule further comprises a stability inducing motif at its 3′-end.

Therefore, in one aspect, the present invention relates to a nucleic acid molecule, comprising:
a. a nucleic acid sequence encoding the peptide sequence X₁₀X₁₁X₁₂X₁₃RX₁₄X₁₅X₁₆X₁₇ (SEQ ID NO: 29), or a sequence at least 70%, 80%, 90% or 95% identical thereto, wherein
   X₁₀ is Threonine, Isoleucine, Phenylalanine, Asparagine, Arginine, Glutamine or Valine,
   X₁₁ is Glycine, Asparagine, Alanine, Leucine or Methionine,
   X₁₂ is Threonine or Proline,
   X₁₃ is Alanine, Tryptophan, Glycine, Proline, Histidine, Asparagine, Aspartic acid, Glutamic acid, Lysine or Serine, R is Arginine,
   X₁₄ is Serine, Glycine or Threonine,
   X₁₅ is Leucine, Valine, Glutamine or Methionine,
   X₁₆ is Tryptophan, Tyrosine, Alanine, Isoleucine, Leucine, Phenylalanine, Valine, Proline, Cysteine or Glycine, and
   X₁₇ is Leucine, Phenylalanine or Isoleucine,
   and
b. an endoplasmic reticulum (ER)-targeting sequence.

In a preferred embodiment the nucleic acid molecule further comprises a stability inducing motif at its 3′-end.

As described in more detail herein, the peptide sequence X₁₀X₁₁X₁₂X₁₃RX₁₄X₁₅X₁₆X₁₇ (SEQ ID NO: 29) encoded by the nucleic acid molecule, relates to a human cytomegalovirus (HCMV) UL40 signal peptide and sequence variants thereof. The HCMV UL40 signal peptide preferably contains a 9-mer sequence. However, HLA-E has been shown to also bind 10-mers. The UL40 peptide preferably has a sequence identity to endogenous HLA-E-binding peptides. The peptide of the invention encoded by the nucleic acid molecule may be referred to as an HLA-E binding peptide, an HCMV UL40 signal peptide or UL40 peptide, or a variant thereof.

The sequence variation encompassed within SEQ ID NO 29 has been demonstrated to not lead to a disruption in biological function, including for example activation of lymphocytes, such as NKG2C+ NK cells and/or T cells resulting in expansion and/or activation of various effector functions. The nucleic acid of the invention, encoding a functional peptide defined for example by SEQ ID NO 29 or other variants thereof including SEQ ID NO 1, is capable therefore of upregulation and stabilization of HLA-E, and accordingly achieve activation of NKG2C+ NK cells and a corresponding therapeutic effect.

In embodiments, the nucleic acid sequence encoding X₁₀X₁₁X₁₂X₁₃RX₁₄X₁₅X₁₆X₁₇ (SEQ ID NO: 29) comprises or consists of a nucleic acid sequence encoding VMAPRTLXL (SEQ ID NO: 1), wherein X is an amino acid with a hydrophobic side chain selected from A, I, L, F, V, P and G.

The present invention is based on the surprising finding that nucleic acid molecules of the present invention are capable of inducing the activation and/or expansion of lymphocytes, such as NKG2C+ NK cells and/or T cells.

In particular, presentation of the peptides encoded by the nucleic acid molecules of the present invention, or fragments thereof, by a non-classical MHC class I molecule, such as preferably HLA-E, leads to the activation of lymphocytes, such as NKG2C+ NK cells and/or T cells resulting in expansion and/or activation of various effector functions. Such functions may include, without being bound by theory, an induction of cell death of the cell presenting the nucleic acid-encoded peptide of the present invention on the non-classical MHC class I molecule, preferably HLA-E, and secretion and/or expression of e.g., TNF-alpha, IFN-gamma, CCL3 and/or CD107a.

The inventors have previously shown that HLA-E stabilizing peptides from the UL40 protein of different HCMV strains are recognized by NKG2C⁺ NK cells, inducing activation and proliferation of NKG2C⁺ NK cells, with the peptide sequence VMAPRTLFL (deriving from rare CMV strains or from the leader sequence of HLA-G) being most efficient¹⁴. The inventors also demonstrated that peptide-specific activation of NKG2C⁺ NK cells in combination with pro-inflammatory cytokines induces their expansion and transcriptional remodeling¹⁴.

As described in more detail below, a nucleic acid molecule, for example encoding the peptide sequence X₁₀X₁₁X₁₂X₁₃RX₁₄X₁₅X₁₆X₁₇ (SEQ ID NO:29) or other preferred sequences as disclosed herein, together with an endoplasmic reticulum (ER)-targeting sequence, and preferably a stability inducing motif at its 3′-end, represents effective means to express the UL40 peptides or derivatives thereof, induce upregulation and stabilization of HLA-E, and accordingly achieve activation of NKG2C⁺ NK cells and the corresponding therapeutic effect. Sequence variation has also been assessed and shown to not cause disruption in the inventive therapeutic properties of the present invention.

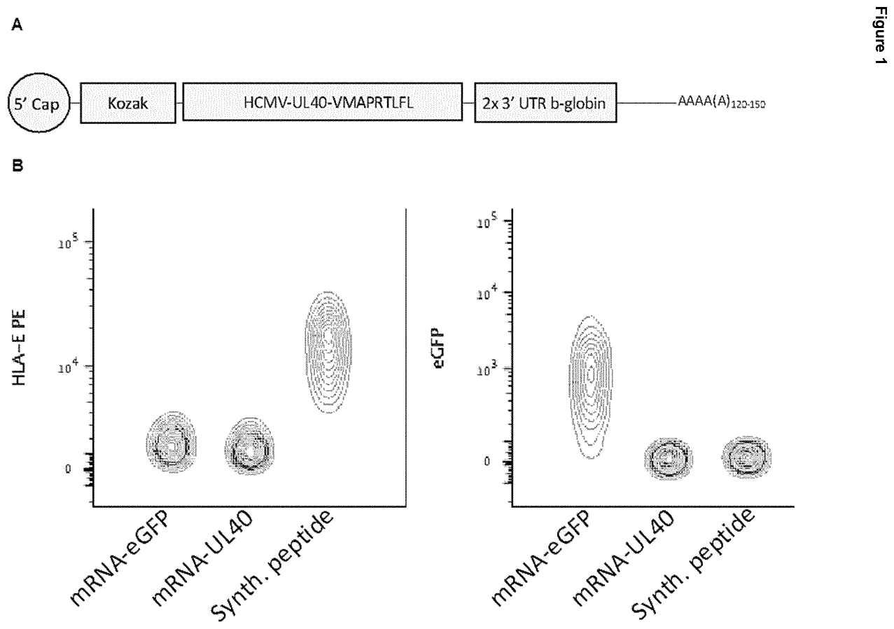

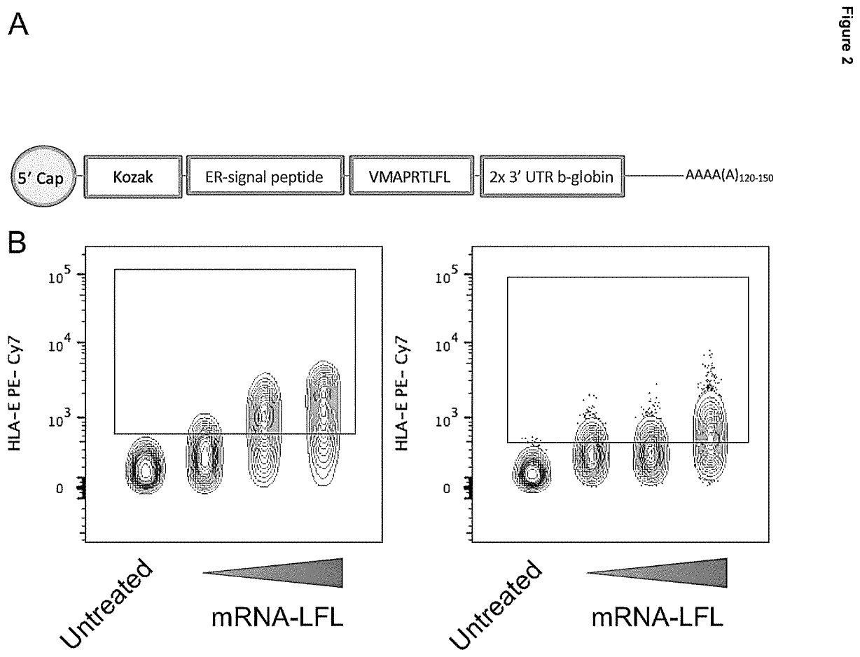

The nucleic acid molecules of the present invention are based on surprising and beneficial effects, as demonstrated in the Examples below. The inventors have previously developed methods involving HLA-E stabilizing target peptides from the UL40 protein of HCMV to stimulate and activate NKG2C⁺ NK cells¹⁴ and EP3539553A1. However, the use of nucleic acid molecules encoding target peptides to be presented on HLA-E for the activation of immune cells has not been described before. When developing the present approach, the inventors surprisingly found that early prototype constructs encoding the full UL40 protein or derivatives thereof did not result in the upregulation of HLA-E on the surface of K562/HLA-E cells, despite efficient expression and delivery of an analogous control mRNA, see, e.g., FIG. 1. These results were entirely unexpected, as previous studies delivering nucleic acids encoding the UL40 protein in recombinant adenoviral vectors had demonstrated upregulation of HLA-E in human fibroblasts [10.1126/science.287.5455.1031]. Further, state of the art methods described for HLA-A presentation in similar systems, such as K562 cells, successfully deliver nucleic acids encoding full length proteins [10.1016/j.imbio.2014.03.003], while the inventors found that additional requirements apply for efficient expression and processing for HLA-E presentation upon transient delivery of nucleic acid molecules.

To solve this problem, the inventors designed new constructs for the expression, processing and delivery of the isolated VMAPRTLFL target peptide and variants thereof into the ER, to be presented on HLA-E and induce the activation of NKG2C⁺ NK cells. For the efficient presentation of the peptide, a signal sequence targeting the ER was placed in front of the peptide-encoding sequence to shuttle it into the ER, where the VMAPRTLFL peptide was cleaved off and loaded onto HLA-E. The incorporation of an endoplasmic reticulum (ER)-targeting sequence produced effective delivery, expression and HLA-E presentation of the peptide, thus effectively solving the unexpected problem of poor HLA-E upregulation and peptide presentation in earlier constructs. Such an ER-exclusive mechanism has not been described before for the HLA-A presentation of mRNA-derived target peptides and ER-targeting is not considered essential for presenting mRNA-derived antigens on HLA-A. Recent post-published studies confirm this observation, describing that unlike classical HLA class I (e.g., HLA-A), which rapidly exits the endoplasmic reticulum (ER) after synthesis, HLA-E is largely retained and accumulates within the ER⁶⁴.

Such efficient translocation of the peptide encoding sequence disclosed herein into the ER protects the peptide encoding sequence from degradation pathways, prolonging its half-life and beneficially increases its efficacy. Cleaving of the signal peptide in the ER is a crucial and highly specific process that ensures that only the desired peptide is released and presented and efficiently ensured by the disclosed peptide encoding sequence for the target peptide. This also minimizes the risk of by-products, off-target products or unwanted immunological reactions. By using the disclosed peptide-encoding sequence, the expression of the target peptide can also be advantageously timed that is essential for its therapeutic application.

It has taken a large number of experiments and the synthesis of various peptide coding sequences to obtain a nucleic acid sequence disclosed herein that satisfies these conditions and has the special features described herein.

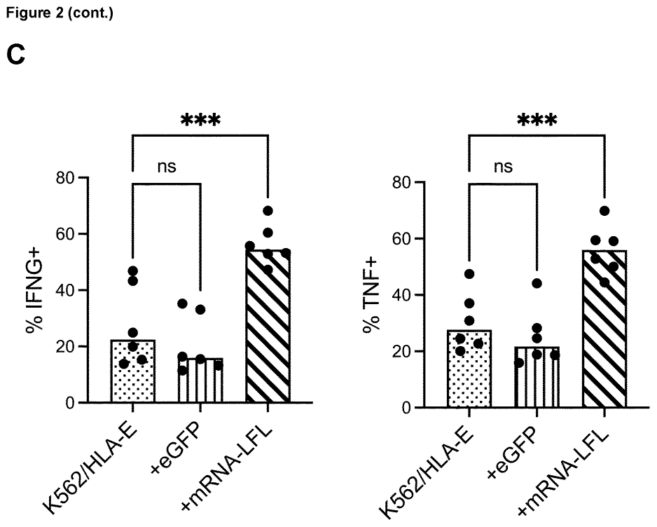

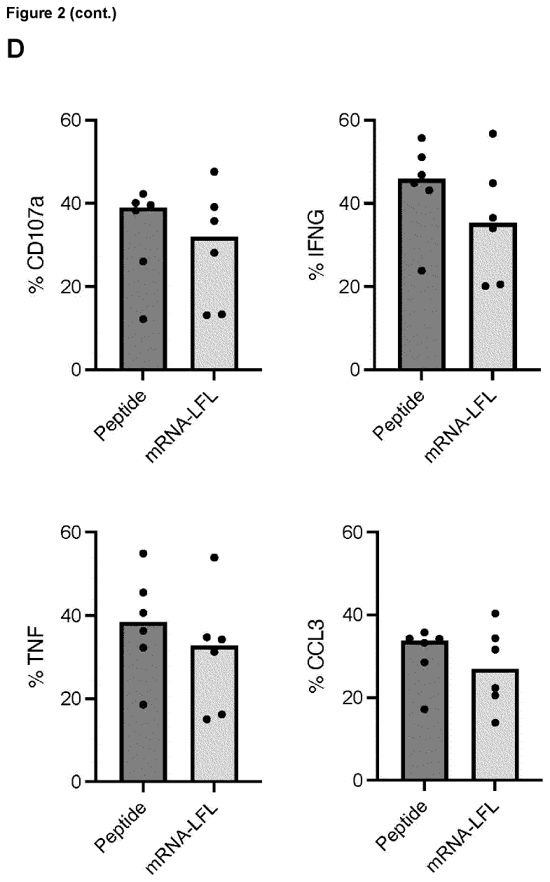

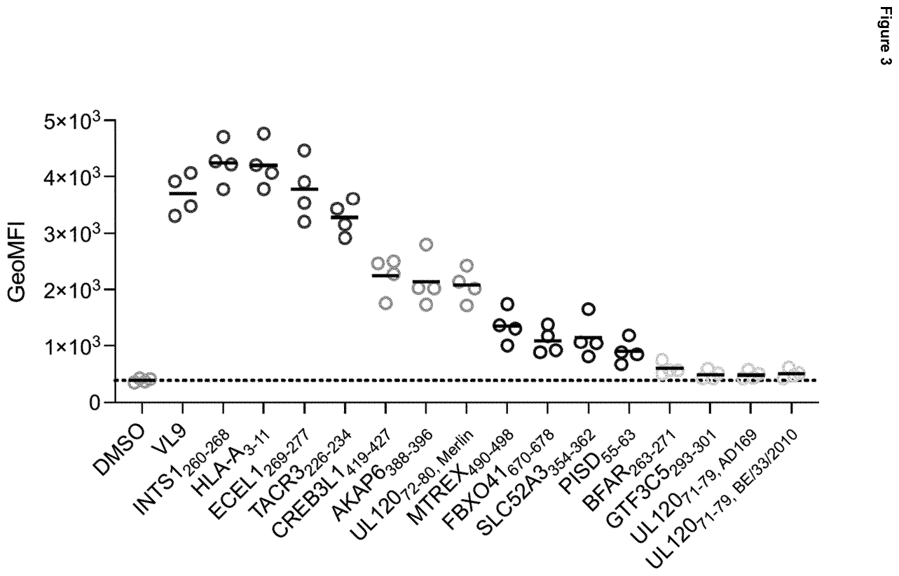

Moreover, the inventors surprisingly found that the present invention enables induction of an efficient and prolonged upregulation of HLA-E on cells, as e.g., shown in Example 1 and FIG. 2B. In their experiments, e.g., see present Examples, the inventors revealed that the present invention facilitates HLA-E stabilization for at least 24 hours after treatment. These results evidence prolonged presentation of a peptide encoded by a nucleic acid sequence according to the invention in comparison to a prior art synthetic peptides¹⁴ and EP3539553A1, which was not detectable anymore after only 6 hours¹⁴ in earlier studies of the inventors. Therefore, this effect of prolonged presentation was highly surprising, because peptides expressed from introduced mRNA do not necessarily have a longer half-life at presentation than introduced peptides. The improved presentation of the peptide encoded by a nucleic acid sequence according to the invention to the cell surface leads to a stronger, longer and more specific immune response than the prior art and the skilled person could not expect.

In embodiments of the invention, the ER-targeting sequence comprises or consists of a nucleic acid sequence of a mouse mammary tumor virus envelope gene. In embodiments, the ER-targeting sequence comprises or consists of a nucleic acid sequence of a mouse mammary tumor virus (MMTV) gp70 envelope gene. In embodiments, the ER-targeting sequence comprises or consists of a sequence according to any one of SEQ ID NO: 47-48.

In embodiments, other suitable ER-targeting sequences are envisaged. In general, signal sequences (e.g., ER-targeting sequences) commonly comprise a generalizable tripartite structure comprising an n-region, a h-region (hydrophobic core), and a c-region. Therefore, starting from the teachings of the current invention and the present disclosure, a skilled person could without undue effort select appropriate signal peptide sequences that may be used alternatively to the targeting sequences disclosed herein (e.g., as comprised within SEQ ID NO: 47-48) from established sources, such as the signal peptide database (http://www.signalpeptide.de/) or using state-of-the-art models to predict signal peptides and their cleavage sites (e.g., according to DOI: 10.1038/s41587-021-01156-3⁶⁸).

In preferred embodiments, a molecule according to the invention would additionally comprise one or more enzyme cleavage sites, such as an (ER-associated) signal-peptidase cleavage site between the ER-targeting sequence and the sequence encoding the peptide of interest, such that the (“bio-active”) peptide of interest, can be cleaved and released from the ER-targeting sequence after delivery to the ER, e.g., such that the encoded peptide can be loaded onto HLA-E.

As shown in the examples below, these particular embodiments surprisingly enable improved biological properties of the inventive nucleic acid and expressed peptide, by effectively inducing upregulation and stabilization of HLA-E. To the knowledge of the inventors, the present invention represents the first successful showing of nucleic acid administration and expression of a UL40 peptide, and it could not have been reasonably expected that using an ER-targeting sequence in the nucleic acid would successfully enable this approach.

Until now, mostly the presentation of antigens via HLA-A has been described and tested in the context of nucleic acid-based expression of target proteins. For example, some prior art studies⁶⁵ used DNA-based anti-cancer vaccines comprising an ER-targeting sequence and a tumor-associated antigen containing B cell epitopes and HLA-A2-presented T cell epitopes to elicit an anti-cancer immune response. In said studies, DNA vaccine-expressed proteins also elicited an antibody-dependent cellular cytotoxicity, comprising the production of antibodies against respective cancer cells marking them for destruction by immune effector cells, e.g., NK cells.

However, the use of a nucleic acid-based construct encoding a short peptide to be presented on HLA-E has not been described before for eliciting an immune response, preferably involving NKG2C+ NK cells. Further, HLA-E is very restrictive regarding the peptides finally being presented on the cellular surface^{66,67}. Hence, the inventors had to develop specific constructs, such as mRNA, expressing a peptide which would, after expression and possible processing, be finally presented by HLA-E. In the state of the art⁶⁵, mostly DNA constructs expressing entire proteins have been described, which could subsequently only be presented by HLA-A or other classical HLA class molecules, as these are less restrictive regarding the sequence of peptides to be presented. The peptide-encoding sequence disclosed herein provides higher biocompatibility, can be advantageously synthesized and can thus be easily adapted to different production volumes, which is advantageous for industrial applications.

In a preferred embodiment the nucleic acid molecule further comprises a stability inducing motif at its 3′-end. In embodiments, the stability inducing motif comprises or consists of at least one copy of a three prime untranslated region (3′UTR) of a beta-globin gene.

In embodiments the beta-globin gene is derived from the group comprising globin genes, such as alpha2-globin, alpha1-globin and beta-globin, preferably human beta-globin gene or a nuclear acid sequence derived therefrom. In specific embodiments the stability inducing motif comprises or consists of a nucleic acid sequence according to any one of SEQ ID NO: 49-52.

In embodiments of the nucleic acid molecule according to the invention the nucleic acid molecule is an RNA molecule, preferably a single-stranded RNA molecule. In embodiments of the nucleic acid molecule according to the invention the nucleic acid molecule is an mRNA (messenger RNA) molecule.

In embodiments of the nucleic acid molecule according to the invention, the nucleic acid sequence encoding VMAPRTLXL and/or X₁₀X₁₁X₁₂X₁₃RX₁₄X₁₅X₁₆X₁₇ comprises or consists of a nucleic acid sequence encoding any one of VMAPRTLFL (SEQ ID NO: 2), VMAPRTLAL (SEQ ID NO: 3), VMAPRTLIL (SEQ ID NO: 4), VMAPRTLLL (SEQ ID NO: 5), VMAPRTLVL (SEQ ID NO: 6), VMAPRTLPL (SEQ ID NO: 7), VMAPRTLGL (SEQ ID NO: 8), or a sequence at least 70%, 80%, 90% or 95% identical thereto.

In embodiments of the nucleic acid molecule according to the invention, the nucleic acid sequence encoding VMAPRTLXL and/or X₁₀X₁₁X₁₂X₁₃RX₁₄X₁₅X₁₆X₁₇ comprises or consists of a nucleic acid sequence encoding any one of VMAPRTLFL (SEQ ID NO: 2), VMAPRTLAL (SEQ ID NO: 3), VMAPRTLIL (SEQ ID NO: 4), VMAPRTLLL (SEQ ID NO: 5), VMAPRTLVL (SEQ ID NO: 6), VMAPRTLPL (SEQ ID NO: 7), VMAPRTLGL (SEQ ID NO: 8), VMAPRTVLL (SEQ ID NO: 9), or VMAPRALLL (SEQ ID NO: 10), or a sequence at least 70%, 80%, 90% or 95% identical thereto.

In embodiments, the nucleic acid sequence comprising the ER-targeting sequence and the stability inducing motif comprises or consist of a nucleic acid sequence according to SEQ ID NO 11-26 respectively, or a sequence at least 70%, 80%, 90% or 95% identical thereto.

In some embodiments of the invention, wherein the nucleic acid molecule is or comprises an RNA molecule, the nucleic acid sequence according to any one or more of SEQ ID NO 11-26 is the respective RNA-sequence corresponding thereto, e.g., wherein thymine (T) is uracil (U).

In one embodiment, the nucleic acid sequence comprising the ER-targeting sequence and the stability inducing motif comprises or consist of a nucleic acid sequence according to SEQ ID NO 26, or a sequence at least 70%, 80%, 90% or 95% identical thereto.

The nucleic acid molecule according to any one of the preceding claims, wherein the nucleic acid sequence comprising the ER-targeting sequence and the stability inducing motif comprises or consist of a nucleic acid sequence according to SEQ ID NO 26, 47, 49-52, respectively.

In embodiments, the nucleic acid molecule comprises a stop codon before its 3′ UTR, namely 3′ (downstream) of the nucleic acid sequence encoding X₁₀X₁₁X₁₂X₁₃RX₁₄X₁₅X₁₆X₁₇ and 5′ (upstream) of the stability inducing motif.

In embodiments, the nucleic acid molecule comprises a cleavage site 5′ (upstream) of the nucleic acid sequence encoding VMAPRTLXL, wherein the cleavage site is preferably an enzyme cleavage site for an ER-associated peptidase.

In embodiments of the nucleic acid molecule according to the invention, the nucleic acid molecule comprises a 5′-cap structure. In embodiments of the nucleic acid molecule according to the invention, the nucleic acid molecule comprises a 5′-cap structure that is selected from the group comprising 7-methylguanosine (m7G) through a 5′-5′-triphosphate bridge (m7GpppN) or with additional methylation at the first nucleotide (m7GpppNmpN) or both first and second nucleotides (m7GpppN^{m}pN^{m}). The 5′-cap structure preferably enhances expression of the nucleic acid molecule and/or preferably avoids innate immune recognition thereof. In embodiments, capping may be achieved enzymatically, e.g., using vaccinia virus capping enzyme, or co-transcriptionally by adding cap analogs, such as, for example, anti-reverse cap analog (ARCA) or, for example, “CleanCaps” co-transcriptional capping provided by CleanCap AG, Germany.

In embodiments, the nucleic acid molecule further comprises a spacer sequence between the nucleic acid sequence encoding VMAPRTLXL and the stability inducing motif and/or the ER-targeting sequence. In embodiments, the spacer sequence preferably encodes a cleavage site, more preferably an enzyme cleavage site for an ER-associated peptidase. The spacer sequence preferably is or comprises a nucleic acid sequence.

In embodiments the nucleic acid sequence encoding the peptide sequence VMAPRTLFL (SEQ ID NO: 2) comprises or consists of a nucleic acid sequence: gtg atg gog ccg cgc acc ctg ttt ctg (SEQ ID NO: 11), gtg atg gcc cct aga acc ctg ttc ctg tga (SEQ ID NO: 12), or gtn atg gen con mgn acn ytn tty ytn (SEQ ID NO: 13), wherein n is g, a, t or c, wherein m is a or c and wherein y is t or c, or a sequence at least 70%, 80%, 90% or 95% identical thereto.

In embodiments the nucleic acid sequence encoding the peptide sequence VMAPRTLAL (SEQ ID NO: 3) comprises or consists of a nucleic acid sequence: gtg atg gcg ccg cgc acc ctg gcg ctg (SEQ ID NO:14) or gtn atg gen con mgn acn ytn gen ytn (SEQ ID NO: 15), wherein n is g, a, tor c, wherein m is a or c and wherein y is t or c, or a sequence at least 70%, 80%, 90% or 95% identical thereto.

In embodiments the nucleic acid sequence encoding the peptide sequence VMAPRTLIL (SEQ ID NO: 4) comprises or consists of a nucleic acid sequence: gtg atg geg ccg cgc acc ctg att ctg (SEQ ID NO:16) or gtn atg gen con mgn acn ytn ath ytn (SEQ ID NO:17), wherein n is g, a, t or c, wherein y is t or c, wherein m is a or c and wherein h is a, tor c, or a sequence at least 70%, 80%, 90% or 95% identical thereto.

In embodiments the nucleic acid sequence encoding the peptide sequence VMAPRTLLL (SEQ ID NO: 5) comprises or consists of a nucleic acid sequence: gtg atg gog ccg cgc acc ctg ctg ctg (SEQ ID NO:18) or gtn atg gen con mgn acn ytn ytn ytn (SEQ ID NO:19), wherein n is g, a, tor c, wherein m is a or c and wherein y is t or c, or a sequence at least 70%, 80%, 90% or 95% identical thereto.

In embodiments the nucleic acid sequence encoding the peptide sequence VMAPRTLVL (SEQ ID NO: 6) comprises or consists of a nucleic acid sequence: gtg atg gog ccg cgc acc ctg gtg ctg (SEQ ID NO: 20) or gtn atg gen con mgn acn ytn gtn ytn (SEQ ID NO:21), wherein n is g, a, tor c, wherein m is a or c and wherein y is t or c, or a sequence at least 70%, 80%, 90% or 95% identical thereto.

In embodiments the nucleic acid sequence encoding the peptide sequence VMAPRTLPL (SEQ ID NO: 7) comprises or consists of a nucleic acid sequence: gtg atg geg ccg cgc acc ctg ccg ctg (SEQ ID NO:22) or gtn atg gen con mgn acn ytn con ytn (SEQ ID NO:23), wherein n is g, a, t or c, wherein m is a or c and wherein y is t or c, or a sequence at least 70%, 80%, 90% or 95% identical thereto.

In embodiments the nucleic acid sequence encoding the peptide sequence VMAPRTLGL (SEQ ID NO: 8) comprises or consists of a nucleic acid sequence: gtg atg gog ccg cgc acc ctg ggc ctg (SEQ ID NO:24) or gtn atg gen con mgn acn ytn ggn ytn (SEQ ID NO:25), wherein n is g, a, tor c, wherein m is a or c and wherein y is t or c, or a sequence at least 70%, 80%, 90% or 95% identical thereto.

In embodiments of the invention, wherein the nucleic acid molecule is or comprises an RNA molecule, any one of the herein disclosed nucleic acid sequences is the respective RNA-sequence corresponding thereto, e.g., wherein thymine (t) is uracil (u). For example, the DNA sequence gtg atg gog ccg cgc acc ctg ttt ctg (SEQ ID NO: 11) also incorporates the RNA sequence gug aug gcg ccg cgc acc cug uuu cug; and the DNA sequence gtg atg gog ccg cgc acc ctg att ctg (SEQ ID NO:16) also incorporates the RNA sequence gug aug geg ccg cgc acc cug auu cug, etc. Therefore, any DNA sequence disclosed herein also discloses the RNA or mRNA sequence corresponding thereto. Moreover, the complementary or reverse complementary sequences of the herein disclosed nucleic acid sequences are also envisaged.

In embodiments of the nucleic acid molecule according to the invention, the nucleic acid sequence comprises or consists of a nucleic acid sequence encoding X₁X₂PX₃RSLX₄X₅ (SEQ ID NO: 27), wherein each X is an amino acid, wherein P is proline, R is Arginine, S is serine, L is Leucine, X₁ is Threonine or Valine, X₂ is Glycine or Asparagine, X₃ is Tryptophan or Glycine, X₄ is Tryptophan or Phenylalanine, and X₅ is Leucine or Isoleucine. The nucleic acid molecule according to the invention may in embodiments comprise or consist of a nucleic acid sequence encoding any of the peptides with the SEQ ID NOs: 1-10, 27-34, and 36-46 and 48, as listed in Table 1.

In embodiments of the nucleic acid molecule according to the invention, the nucleic acid sequence comprises or consists of a nucleic acid sequence encoding X₁X₆PX₇RX₈X₉FL (SEQ ID NO: 28), or a sequence at least 70%, 80%, 90% or 95% identical thereto, wherein each X is an amino acid, wherein P is proline, R is Arginine, F is Phenylalanine, L is Leucine, X₁ is Threonine or Valine, X₆ is Alanine or Asparagine, X₇ is Alanine or Glycine, X₈ is Serine or Threonine, and X₉ is Leucine or Methionine. The nucleic acid molecule according to the invention may in embodiments comprise or consist of a nucleic acid sequence encoding any of the peptides with the SEQ ID NOs: 1-10, 27-34, and 36-46 and 48, as listed in Table 1.

In embodiments of the nucleic acid molecule according to the invention, the nucleic acid sequence comprises or consists of a nucleic acid sequence encoding any one of QMPSRSLLF (SEQ ID NO: 30), TLPKRGLFL (SEQ ID NO: 31), TGPWRSLWI (SEQ ID NO: 32), VNPGRSLFL (SEQ ID NO: 34), TLPERTLYL (SEQ ID NO: 36), FLPNRSLLF (SEQ ID NO: 37), VMPPRTLLL (SEQ ID NO: 40), VMPGRTLCF (SEQ ID NO: 41), RMPPRSVLL (SEQ ID NO: 42), TAPARTMFL (SEQ ID NO: 43), NMPARTVLF (SEQ ID NO: 44), VLPHRTQFL (SEQ ID NO: 45) and LTDRSLWL (SEQ ID NO: 46), or a sequence at least 70%, 80%, 90% or 95% identical thereto.

Without being bound by theory, in some embodiments the nucleic acid molecule encodes a peptide capable of binding to both NKG2A and NKG2C, resulting in activation of NKG2C+ cells and preferably in inhibition of NKG2A+ cells. For example, the nucleic acid molecules encoding the peptides of any one of SEQ ID NO: 1-10 or 27-34, and 36-46 induce inhibitory and activating effects on NKG2A+/NKG2C− and NKG2A-/NKG2C+ cells, respectively.

Without being bound by theory, in some embodiments, each of the peptides disclosed herein may be able to modulate the activities of T cells, preferably in particular of HLA-E-restricted CD8+ T cells, in addition or alternative to NK cells. This effect is considered plausible in view of the earlier findings of the inventors, as described in Pietra et al.⁶².

In embodiments, one or more of the above functions are considered as features for combination with, and defining for, potential sequence variation leading to sequences of at least 70%, 80%, 90% or 95% identity, to any specific sequence disclosed herein.

In preferred embodiments the nucleic acid molecule comprises a nucleic acid sequence encoding the peptide sequence X₁₀X₁₁X₁₂X₁₃RX₁₄X₁₅X₁₆X₁₇ (SEQ ID NO: 29) or a sequence at least 70%, 80%, 90% or 95% identical thereto, wherein X₁₀ is Threonine, Isoleucine, Phenylalanine, Asparagine, Arginine, Glutamine or Valine, X₁₁ is Glycine, Asparagine, Alanine, Leucine or Methionine, X₁₂ is Threonine or Proline, X₁₃ is Alanine, Tryptophan, Glycine, Proline, Histidine, Asparagine, Aspartic acid, Glutamic acid, Lysine or Serine, R is Arginine, X₁₄ is Serine, Glycine or Threonine, X₁₅ is Leucine, Valine, Glutamine or Methionine, X₁₆ is Tryptophan, Tyrosine, Alanine, Isoleucine, Leucine, Phenylalanine, Valine, Proline, Cysteine or Glycine, and X₁₇ is Leucine, Phenylalanine or Isoleucine. The peptides encoded by the nucleic acid molecule according to the invention may in embodiments comprise any of the peptides with the SEQ ID NOs: 1-10, 27-34, and 36-46 and 48, as listed in Table 1.

In embodiments of the nucleic acid molecule according to the invention the nucleic acid sequence encoding X₁₀X₁₁X₁₂X₁₃RX₁₄X₁₅X₁₆X₁₇ comprises or consists of a nucleic acid sequence encoding any one of VMAPRTLFL (SEQ ID NO: 2), VMAPRTLAL (SEQ ID NO: 3), VMAPRTLIL (SEQ ID NO: 4), VMAPRTLLL (SEQ ID NO: 5), VMAPRTLVL (SEQ ID NO: 6), VMAPRTLPL (SEQ ID NO: 7), VMAPRTLGL (SEQ ID NO: 8), QMPSRSLLF (SEQ ID NO: 30), TLPKRGLFL (SEQ ID NO: 31), TGPWRSLWI (SEQ ID NO: 32), ILTDRSLWL (SEQ ID NO: 33), VNPGRSLFL (SEQ ID NO: 34), TLPERTLYL (SEQ ID NO: 36), FLPNRSLLF (SEQ ID NO: 37), VMPPRTLLL (SEQ ID NO: 40), VMPGRTLCF (SEQ ID NO: 41), RMPPRSVLL (SEQ ID NO: 42), TAPARTMFL (SEQ ID NO: 43), NMPARTVLF (SEQ ID NO: 44), VLPHRTQFL (SEQ ID NO: 45) and LTDRSLWL (SEQ ID NO: 46), or a sequence at least 70%, 80%, 90% or 95% identical thereto.

In embodiments of the nucleic acid molecule according to the invention the nucleic acid sequence encoding X₁₀X₁₁X₁₂X₁₃RX₁₄X₁₅X₁₆X₁₇ comprises or consists of a nucleic acid sequence encoding any one of VMAPRTLFL (SEQ ID NO: 2), VMAPRTLAL (SEQ ID NO: 3), VMAPRTLIL (SEQ ID NO: 4), VMAPRTLLL (SEQ ID NO: 5), VMAPRTLVL (SEQ ID NO: 6), VMAPRTLPL (SEQ ID NO: 7), VMAPRTLGL (SEQ ID NO: 8), VMAPRTVLL (SEQ ID NO: 9), VMAPRALLL (SEQ ID NO: 10), QMPSRSLLF (SEQ ID NO: 30), TLPKRGLFL (SEQ ID NO: 31), TGPWRSLWI (SEQ ID NO: 32), ILTDRSLWL (SEQ ID NO: 33), VNPGRSLFL (SEQ ID NO: 34), TLPERTLYL (SEQ ID NO: 36), FLPNRSLLF (SEQ ID NO: 37), VMPPRTLLL (SEQ ID NO: 40), VMPGRTLCF (SEQ ID NO: 41), RMPPRSVLL (SEQ ID NO: 42), TAPARTMFL (SEQ ID NO: 43), NMPARTVLF (SEQ ID NO: 44), VLPHRTQFL (SEQ ID NO: 45) and LTDRSLWL (SEQ ID NO: 46), or a sequence at least 70%, 80%, 90% or 95% identical thereto.

In embodiments the nucleic acid molecule according to the invention comprises or consists of a nucleic acid sequence encoding any one of VMAPRTLFL (SEQ ID NO: 2), VMAPRTLAL (SEQ ID NO: 3), VMAPRTLIL (SEQ ID NO: 4), VMAPRTLLL (SEQ ID NO: 5), VMAPRTLVL (SEQ ID NO: 6), VMAPRTLPL (SEQ ID NO: 7), VMAPRTLGL (SEQ ID NO: 8), QMPSRSLLF (SEQ ID NO: 30), TLPKRGLFL (SEQ ID NO: 31), TGPWRSLWI (SEQ ID NO: 32), ILTDRSLWL (SEQ ID NO: 33), VNPGRSLFL (SEQ ID NO: 34), TLPERTLYL (SEQ ID NO: 36), FLPNRSLLF (SEQ ID NO: 37), VMPPRTLLL (SEQ ID NO: 40), VMPGRTLCF (SEQ ID NO: 41), RMPPRSVLL (SEQ ID NO: 42), TAPARTMFL (SEQ ID NO: 43), NMPARTVLF (SEQ ID NO: 44), VLPHRTQFL (SEQ ID NO: 45) and LTDRSLWL (SEQ ID NO: 46), or a sequence at least 70%, 80%, 90% or 95% identical thereto.

In embodiments the nucleic acid molecule according to the invention comprises or consists of a nucleic acid sequence encoding any one of VMAPRTLFL (SEQ ID NO: 2), VMAPRTLAL (SEQ ID NO: 3), VMAPRTLIL (SEQ ID NO: 4), VMAPRTLLL (SEQ ID NO: 5), VMAPRTLVL (SEQ ID NO: 6), VMAPRTLPL (SEQ ID NO: 7), VMAPRTLGL (SEQ ID NO: 8), VMAPRTVLL (SEQ ID NO: 9), VMAPRALLL (SEQ ID NO: 10), QMPSRSLLF (SEQ ID NO: 30), TLPKRGLFL (SEQ ID NO: 31), TGPWRSLWI (SEQ ID NO: 32), ILTDRSLWL (SEQ ID NO: 33), VNPGRSLFL (SEQ ID NO: 34), TLPERTLYL (SEQ ID NO: 36), FLPNRSLLF (SEQ ID NO: 37), VMPPRTLLL (SEQ ID NO: 40), VMPGRTLCF (SEQ ID NO: 41), RMPPRSVLL (SEQ ID NO: 42), TAPARTMFL (SEQ ID NO: 43), NMPARTVLF (SEQ ID NO: 44), VLPHRTQFL (SEQ ID NO: 45) and LTDRSLWL (SEQ ID NO: 46), or a sequence at least 70%, 80%, 90% or 95% identical thereto.

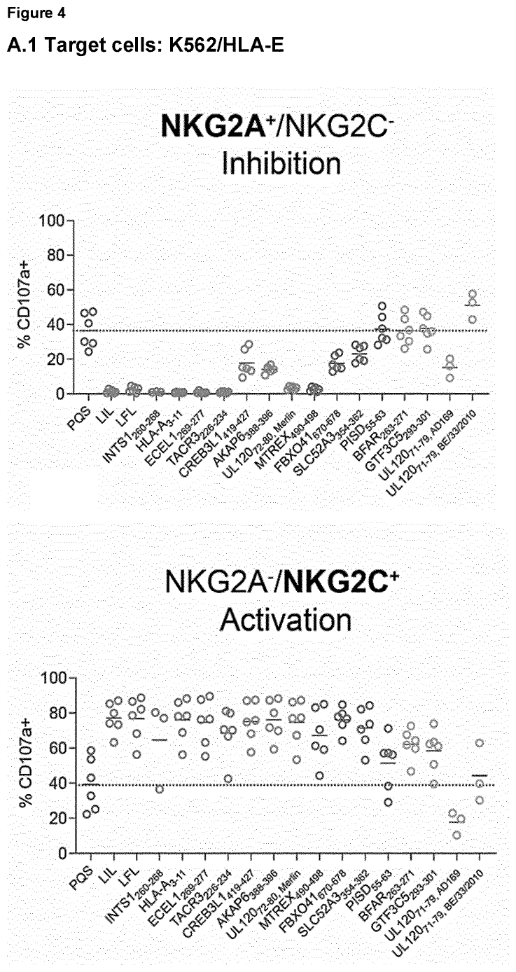

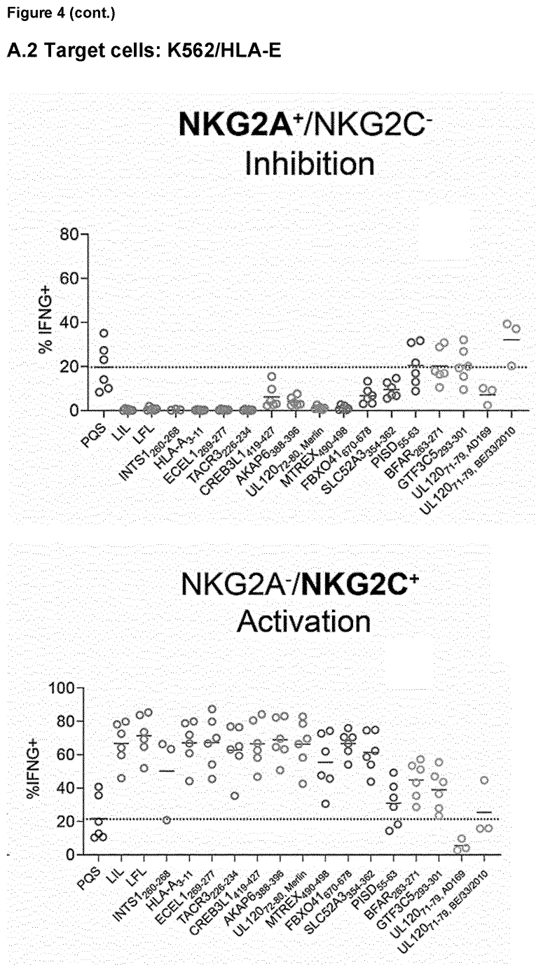

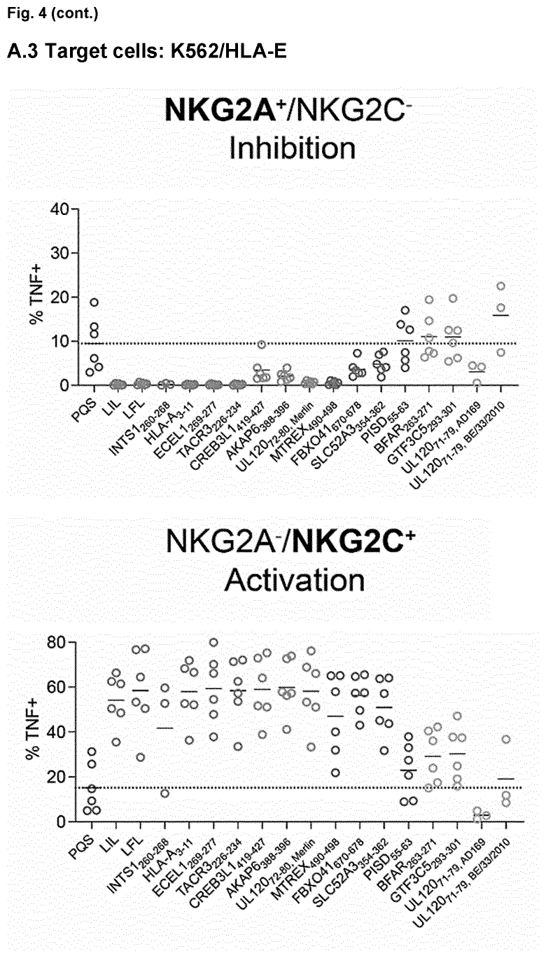

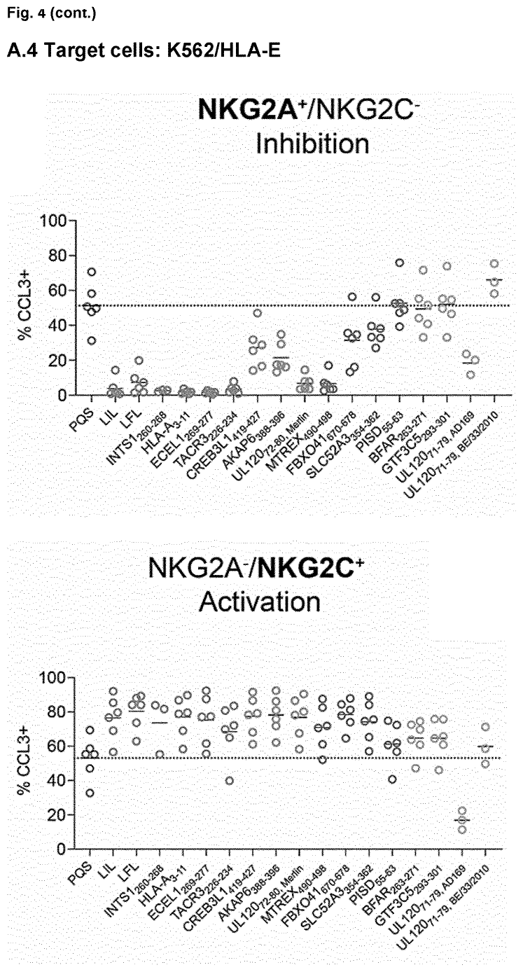

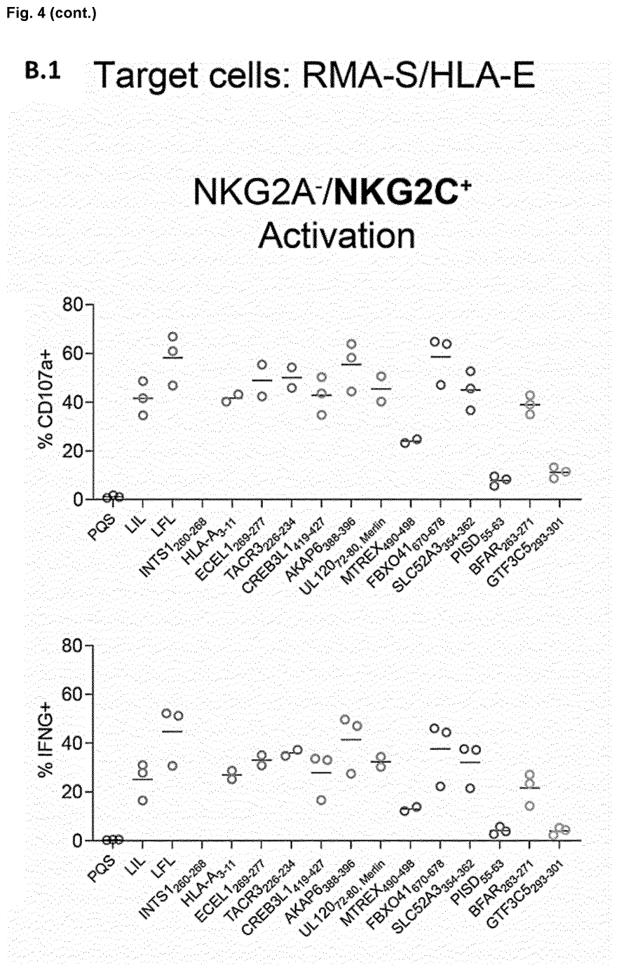

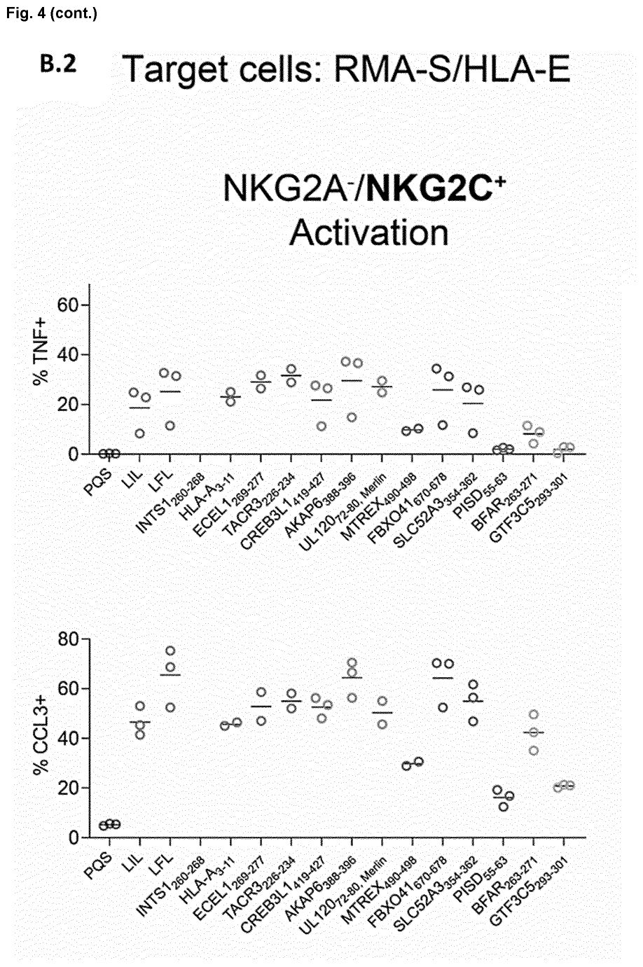

As demonstrated in the examples below, several peptide sequences differing from the presently claimed sequences were assessed (e.g., UBAC2 (SEQ ID NO:35), EMC1 (SEQ ID NO:38), OR5D14 (SEQ ID NO:39), UL120_{71-79AD169 or/BE/33/2010} and PQS (SEQ ID NO:53)) and did not show the relevant activity (refer e.g., FIGS. 3 and 4). The sequences covered by the generic sequence according to SEQ ID NO 29 therefore represent a set of functional peptides shown to have the relevant activity over other related sequences.

In another aspect, the present invention relates to the nucleic acid molecule according to the invention for use as a medicament.

In another aspect, the present invention relates to the nucleic acid molecule according to the invention for use in an immunogenic composition, such as a vaccine, to prevent and/or treat a medical condition associated with a human cytomegalovirus (HCMV) infection.

In embodiments, the invention relates to the nucleic acid molecule described herein for use in an immunogenic composition, such as a vaccine, to inhibit reactivation of human cytomegalovirus (HCMV) infections and/or reduce viral titers in an individual infected with HCMV.

In embodiments, the invention relates to the nucleic acid molecule described herein for use in an immunogenic composition, such as a vaccine, to prevent and/or treat a medical condition associated with a human cytomegalovirus (HCMV) infection and/or to inhibit reactivation of human cytomegalovirus (HCMV) infections and/or reduce viral titers in an individual infected with HCMV.

Although the use of synthetic peptides is a potential strategy to translate the inventors' earlier findings¹⁴ into a therapy, peptide vaccines have had limited success in preventing infectious diseases or as immunotherapeutic interventions to treat malignancies. Potential limitations include low immunogenicity without adjuvants, antigen uptake, and peptide stability^{23,24} In contrast, mRNA immunization approaches have recently emerged as highly efficient tools for immunization²⁷. Therefore, the inventors first developed a prototype mRNA vaccine encoding the HCMV UL40 protein and containing the VMAPRTLFL peptide (“mRNA-UL40”, see FIG. 1A). To stabilize the mRNA and for efficient translation, the inventors placed two copies of the 3′UTR of beta-globin in a head-to-tail orientation at the 3′ end of our construct, as previously described^{27, 28} Surprisingly, this construct did not upregulate HLA-E on the surface of K562/HLA-E cells, although an analogous mRNA encoding an enhanced green fluorescent protein (“mRNA-eGFP”, see FIG. 1B) was efficiently expressed and delivered.

To overcome the limitations of these constructs, the inventors designed a new construct for expression and transport of the peptides according to the invention, exemplified by the construct encoding the VMAPRTLFL peptide, into the ER to present the peptide on HLA-E and induce activation of NKG2C+ NK cells (“mRNA-LFL” encoding a peptide according to a variant of the peptide VMAPRTLXL, FIG. 2A, SEQ ID NO:1). For efficient presentation of the peptide, an ER-targeting signal sequence is placed upstream of the peptide coding sequence to transport it to the ER where it is loaded onto HLA-E.

To avoid the need for further processing in addition to cleavage of the signal sequence, the inventors decided to only include the sequence encoding the VMAPRTLFL peptide without the rest of the UL40 protein. The mRNA was stabilized by incorporation of two copies of the 3′UTR of beta-globin in a head-to-tail orientation, as described above and elsewhere herein. Surprisingly, und importantly, the nucleic acid molecule according to one embodiment of the invention, referred to in the following as “mRNA-LFL” was able to successfully upregulate HLA-E on the surface of K562/HLA-E cells (see FIG. 2B). Stabilization of HLA-E was observed for at least 24 hours after treatment, indicating significantly prolonged peptide presentation compared to synthetic peptide, which was undetectable after only 6 hours. The nucleic acid of the invention therefore enables an unexpected improvement over earlier attempts using peptide-based strategies.

Co-culture of primary human NK cells with K562/HLA-E cells transfected with the nucleic acid molecule of one embodiment of the invention, referred to herein as “mRNA-LFL”, resulted in efficient and specific activation of NKG2C+ NK cells at levels similar to those observed with high concentrations of the synthetic peptide (FIG. 2C). Taken together, these results demonstrate the efficient expression, processing and presentation of nucleic acid molecules according to the invention and strongly support its potential use as a novel mRNA vaccine targeting lymphocytes, such as NKG2C+ NK cells.

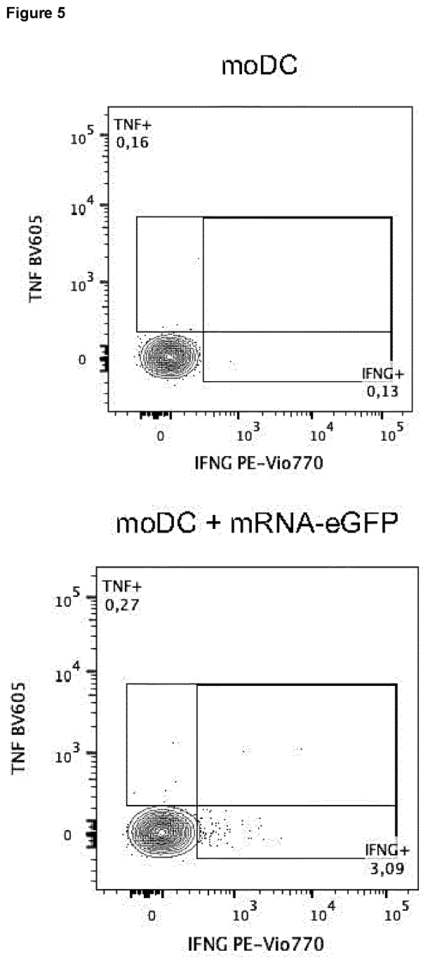

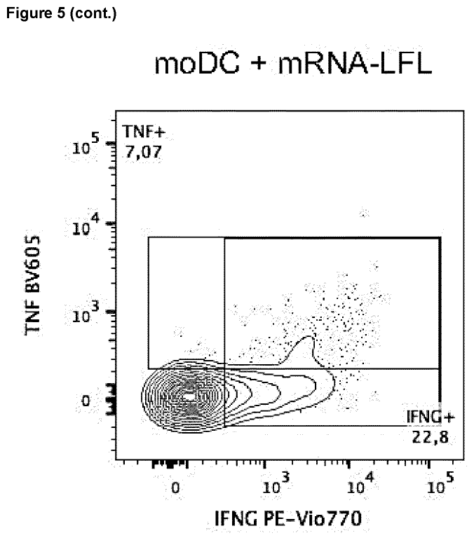

The data presented in Example 1 and FIG. 5 demonstrate the ability of the nucleic acid molecules according to the invention, exemplified by the construct encoding the functional peptide sequence VMAPRTLFL (“mRNA-LFL”), to engage NKG2C⁺ NK cells when translated and processed by autologous, professional antigen-presenting cells, which underlines its functionality under physiological conditions and therefore further supports its therapeutic potential.

These findings of the inventors were entirely surprising. The prior art publication of Lanier et al.³⁰ may describe peptides similar to some of the peptides of interest disclosed herein but does not mention their delivery as an mRNA vaccine. Moreover, as described above, mRNA delivery has major advantages over the delivery of synthetic peptides, such as a prolonged peptide presentation. Furthermore, the inventors unexpectedly found that simply encoding the peptides of interest in a recombinant mRNA, even in their natural protein context, was not enough to effectively achieve the benefits shown for exemplary peptides of the invention (FIG. 1). Instead, the inventors surprisingly found that the respective RNA sequences encoding the peptide sequences of interest had to be taken out of their natural sequence context and had to be placed subsequent to an ER targeting sequence to enable efficient presentation and contacting of NKG2C⁺ NK cells (FIG. 2). The necessity and success of this approach was entirely unexpected from the disclosure of the prior art, but surprisingly overcame the unexpected hurdles the inventors encountered when using RNA sequences corresponding to the peptide sequences disclosed herein and partially in the prior art.

In summary, while the present invention uses an ER targeting sequence, Scheinberg et al. describe a method for identifying epitopes for TCRs. The present invention preferably employs a particularly efficient ER targeting sequence for the delivery of defined peptides into the ER as part of, e.g., the herein disclosed mRNA vaccine, which preferably primarily targets NK cell receptors or TCRs, that are preferably restricted to HLA-E. From these features, significant differences and advantages over the disclosure of the prior art, e.g., of Scheinberg et al.,²⁹, arise, such as an established bioactivity of the present peptides and the encoding mRNAs (e.g., for specific indications, such as, without limitation, for preventing and/or treating a HCMV infection and cancer or other CMV-associated diseases). Moreover, the present molecules are advantageously applicable in a treatment and/or preventative context, such as e.g., as pharmaceutical formulation, e.g., as a vaccine, rather than in the context of an in vitro screening as disclosed in Scheinberg et al.,²⁹. In addition, the molecules according to the invention may in embodiments be used for targeting the non-classical MHC class I molecule HLA-E and show therefore (as shown in the examples herein) activity against NKG2C/A of NK cells (and preferably also on T cells, more preferably on HLA-E-restricted TCRs on T cells). Therefore, the present invention constitutes a significant improvement over the prior art, enabling novel treatment options and applications of the (peptide) sequences disclosed herein.

There are several clinical correlates that point towards NKG2C⁺ memory NK cells in the control of viral infection, in particular of HCMV, and tumor disease. The genetic absence of KLRC2, the gene encoding NKG2C, is a risk factor for HCMV reactivation after lung and hematopoietic stem cell transplantation (HSCT)¹⁵⁻¹⁷. Further, high frequencies of NKG2C⁺ NK cells before kidney transplantation are associated with a reduced incidence of HCMV viremia¹⁸. With regards to tumor control, NKG2C⁺ NK cell expansion has been associated with reduced leukemia relapse risk after HSCT, suggesting that enhanced recognition of HLA-E⁺ blasts might contribute to the control of residual disease¹⁹. The data presented in the examples indicates, without being bound by theory, that one potential mechanism for this effect could be the efficient recognition of HLA-G-derived peptides presented in the context of HLA-E complexes expressed by HLA-E⁺/HLA-G⁺ tumor cells^{20,21} by NKG2C⁺ NK cells. Accordingly, the exploitability of memory NK cells for anti-tumor therapy has been the subject of continuous discussions in the field²². Overall, this suggests that a therapy, which induces NKG2C⁺ memory NK cells and enhances their effector functions, could be effective to treat both HCMV infection, as well as HLA-E⁺/HLA-G⁺ tumors. The combined anti-tumor and anti-viral effects of the present invention could, in embodiments, be especially advantageous in the context of hematopoietic stem cell transplantation after chemotherapy.

Accordingly, the nucleic acid molecules according to the invention can be applied in the context of the prevention and/or treatment of diseases associated with pathogenic cells expressing HLA-E and a peptide of the present invention.

In embodiments the invention relates to the nucleic acid molecule according to the invention for use as a medicament to expand and/or activate NKG2C+ natural killer (NK) cells in the treatment and/or prevention of a medical condition associated with pathogenic cells expressing HLA-E.

In embodiments the invention relates to the nucleic acid molecule described herein for use in an immunogenic composition, such as a vaccine, to expand and/or activate NKG2C+ natural killer (NK) cells in the treatment and/or prevention of a medical condition associated with pathogenic cells expressing HLA-E.

In embodiments, the invention relates to the nucleic acid molecule described herein for use to inhibit reactivation of human cytomegalovirus (HCMV) infections and/or reduce viral titers in an individual infected with HCMV to expand and/or activate NKG2C+ natural killer (NK) cells in the treatment and/or prevention of a medical condition associated with pathogenic cells expressing HLA-E.

In embodiments the invention relates to the nucleic acid molecule described herein for any use described herein to expand and/or activate T-cells in the treatment and/or prevention of a medical condition associated with pathogenic cells expressing HLA-E.

Accordingly, the nucleic acid molecules according to the invention can be used in the context of prevention and/or treating diseases, which are associated with pathogenic cells, which express HLA-E and peptides comprising an amino acid sequence of a peptide of the present invention, for example cells expressing HLA-G or UL-40 of HCMV.

Furthermore, the peptide of the present invention can be used as a medicament to expand and/or activate NKG2C+ natural killer (NK) cells in the treatment and/or prevention of a medical condition associated with pathogenic cells expressing HLA-E and a peptide comprising an amino acid sequence of a peptide of the invention.

In embodiments, the invention relates to the nucleic acid molecule according to the invention for use as a medicament to treat cancer. In embodiments, said cancer expresses HLA-E. In embodiments, the expression of HLA-E is above levels in healthy control cells.

The cancer to be treated may, without limitation, be selected from the group consisting of leukemia, Melanoma, choriocarcinoma, breast cancer, endometrial cancer, ovarian cancer, cervical cancer, esophageal squamous cell carcinoma, colorectal cancer, gastric cancer, hepatocellular carcinoma, glioblastoma, lung cancer, nasopharyngeal carcinoma, pancreatic adenocarcinoma, thyroid carcinoma and renal carcinoma.

In embodiments, the invention relates to the nucleic acid molecule described herein for use in an immunogenic composition, such as a vaccine, to treat and/or prevent cancer, wherein said cancer expresses HLA-E, preferably wherein the expression of HLA-E is above levels in healthy control cells, and wherein the cancer is preferably selected from the group consisting of leukemia, Melanoma, choriocarcinoma, breast cancer, endometrial cancer, ovarian cancer, cervical cancer, esophageal squamous cell carcinoma, colorectal cancer, gastric cancer, hepatocellular carcinoma, glioblastoma, lung cancer, nasopharyngeal carcinoma, pancreatic adenocarcinoma, thyroid carcinoma and renal carcinoma.

In another aspect, the present invention relates to a pharmaceutical composition comprising the nucleic acid molecule described herein, and at least one pharmaceutically acceptable carrier, preferably a carrier enabling intracellular delivery. Preferably, the introduction of a nucleic acid molecule according to the invention into a cell, in particular in an in vivo context, results in the expression of the encoded (poly) peptide or portions thereof in said cell.

In embodiments, the composition is configured for delivery by nanoparticle, lipofection and/or lipo-nanoparticles. In embodiments, a composition configured for delivery of the nucleic acid molecule according to the invention comprises, for example, lipid-containing carrier or vehicles, such as liposomes, cationic lipids, cationic liposomes, and micelles, nanoparticles and lipo-nanoparticles. As an example, cationic lipids can form complexes with the negatively charged nucleic acids. In embodiments carriers enabling intracellular delivery may be selected from the afore-mentioned substances.

In another aspect the present invention relates to an in vitro method for cultivating and/or expanding NKG2C+ natural killer (NK) cells, said method comprising:
a. providing leukocyte cells from a donor, wherein said leukocytes comprise NK cells;
b. contacting said NK cells with a nucleic acid molecule according to the invention; and
c. optionally isolating or enriching NKG2C+ NK cells.

In a further aspect, the invention relates to an isolated population of NKG2C+ natural killer (NK) cells produced by the method according to the invention.

In another aspect, the present invention relates to an in vitro method for cultivating and/or expanding T cells, said method comprising:
a. providing leukocyte cells from a donor, wherein said leukocytes comprise T cells;
b. contacting said T cells with a nucleic acid molecule according to the invention; and
c. optionally isolating or enriching T cells.

This aspect of the invention is based on the surprising finding that cells presenting the complex formed by HLA-E and the peptide encoded by the nucleic acid molecule according to the present invention on their surface are recognized by NKG2C+ NK cells through engagement of the complex with the CD94/NKG2C heterodimer. This leads to activation of the NKG2C+ NK cells resulting in secretion of several effector proteins and induction of cytotoxicity towards the cells presenting the complex. Such cells are in most cases pathogenic cells, such as tumor/cancer cells expressing HLA-G, comprising a peptide of the present invention in its leader/signal sequence, and HLA-E, or HCMV infected cells comprising actively replicating HCMV expressing UL-40. The pathogenic cells can be more efficiently fought by the immune system after recognition by NKG2C+ NK cells. Accordingly, provision of an increased number of NKG2C+ NK cells is beneficial for the treatment of disease associated with pathogenic cells expressing peptides comprising the amino acid sequence of a peptide of the present invention, preferably on HLA-E. Such an increased number of NKG2C+ NK cells can be provided or achieved by administration of the nucleic acid molecule according to the present invention leading to in vivo expansion of the NKG2C+ NK cells, or administration of NKG2C+ NK cells of the present invention, which have been generated by the in vitro method of the present invention.

In embodiments, the nucleic acid molecule according to the invention is used as a medicament to expand and/or activate NKG2C+ natural killer (NK) cells. In embodiments, the nucleic acid molecule according to the invention is used as a medicament to expand and/or activate T cells, preferably HLA-E-restricted CD8+ T cells. T cells, particularly HLA-E-restricted CD8+ T cells, have been demonstrated to recognize similar peptides presented by HLA-E resulting in their proliferation and cytolytic effector functions^{62, 63}.

The present invention is effective in the treatment of diseases associated with pathogenic cells that present peptides according to the invention on HLA-E on their surface. The non-classical MHC class I molecule HLA-G comprises the peptide of the invention in its signal sequence. HLA-G is expressed or upregulated in particular cancer cells. Such cancer cells can be identified by expression analysis of HLA-G and HLA-E, by various methods known to the person skilled in the art.

Expression of HLA-G has been reported for several cancers, which are preferably treated with the peptide of the present invention, which comprise, without limitation, leukemia, Melanoma, choriocarcinoma, breast cancer, endometrial cancer, ovarian cancer, cervical cancer, esophageal squamous cell carcinoma, colorectal cancer, gastric cancer, hepatocellular carcinoma, glioblastoma, lung cancer, nasopharyngeal carcinoma, pancreatic adenocarcinoma, thyroid carcinoma and renal carcinoma. Cancers expressing HLA-G are known to the skilled person and are disclosed in the art (see for example Curigliano G, Criscitiello C, Gelao L, Goldhirsch A. Molecular pathways: human leukocyte antigen G (HLA-G). Clin Cancer Res. 2013; 19(20):5564-71; Lin A, Yan W H. HLA-G expression in cancers: roles in immune evasion, metastasis and target for therapy. Mol Med. 2015; Seliger B, Schlaf G. Structure, expression and function of HLA-G in renal cell carcinoma. Semin Cancer Biol. 2007; 17(6):444-50).

In embodiments of the invention, the nucleic acid molecule according to the invention is used as a medicament to treat a cancer associated with expression of HLA-E, wherein the cancer is identified by
a. providing a sample comprising cancer cells from a patient and
b. determining expression of HLA-E in said sample.

Expression of HLA-E in cancer cells may be determined on the protein or the nucleic acid level. For example, mRNA expression levels of HLA-E encoding mRNA may be determined by qRT-PCR or sequencing analysis, as known to the person skilled in the art (see for example Paul, P., et al. (2000). “HLA-G, -E, -F preworkshop: tools and protocols for analysis of non-classical class/genes transcription and protein expression.” Human Immunology 61(11): 1177-1195). Furthermore, expression may be analyzed on the protein level for example by cytometric analysis of HLA-G and HLA-E expression on the cells surface.

In embodiments, the nucleic acid molecule according to the invention is used as a medicament to treat a cancer associated with elevated expression of HLA-E compared to non-cancerous cells, or a cancer susceptible to NKG2C+ NK cell cytotoxic activity. In the context of the method of the invention, the expression level of HLA-E determined in a sample comprising cancer cells from a subject may be compared to the expression of HLA-E in a reference standard sample and/or in a corresponding sample isolated from a healthy individual, and/or in a corresponding sample that does not comprise cancer cells. A corresponding sample may be a sample that has been isolated form the same tissue or bodily fluid, but does not comprise any cancer cells, for example because it has been isolated from a healthy individual.

In embodiments, the nucleic acid molecule according to the invention is used as a medicament to treat a cancer associated with elevated expression of HLA-E. In further embodiments, the peptide of the invention is used as a medicament to treat a cancer susceptible to NKG2C+ NK cell cytotoxic activity.

In a preferred embodiment of the invention, the nucleic acid molecule according to the invention is used as a medicament to treat leukemia and inhibit reactivation of HCMV infections in subjects having received hematopoietic stem cell transplantation (HSCT). The activation and expansion of NKG2C+ NK cells by peptides encoded by the nucleic acid molecule according to the present invention may be particular advantageous in the context of HSCT, because NK cells are among the first lymphocyte populations to recover after transplantation and therefore can be targeted by the using the approach of the present invention, thus potentially protecting against HCMV reactivation and tumor relapse in leukemia patients after HSCT.

In a further preferred embodiment of the invention, the nucleic acid molecule described herein or the pharmaceutical composition described herein is administered in combination with an adjuvant, preferably selected from an adjuvant enhancing production of or comprising IL-15, IL-12 and/or IL-18. In embodiments, the nucleic acid molecule according to the invention is administered in combination with IL-15, IL-12 and/or IL-18. In preferred embodiments, the adjuvant is inducing or enhancing the production of pro-inflammatory cytokines.

In embodiments, the nucleic acid molecule according to the invention are administered in combination with one or more pro-inflammatory cytokines. It was surprising, that combined stimulation of NKG2C+ NK cells with the complex consisting of HLA-E and the nucleic acid molecule according to the invention and pro-inflammatory cytokines, such as for example IL-15, IL-12 and/or IL-18, induces accumulation of NKG2C+ NK cells, also of NK cells from or in HCMV-individuals.

In preferred embodiments of the invention, the nucleic acid molecule is administered in combination with a check point inhibitor, preferably an inhibitor of an inhibitory receptor selected from the group comprising LILRB1, inhibitory KIRs, NKG2A, PD-1, CTLA-4, TIM-3, TIGIT and LAG-3.

Check point inhibitors have gained much attention in the context of cancer treatment, as it has been found that the inhibition or blockage of inhibitory receptors expressed by immune cells and in particular immune effector cells, such as for example T cells, but also NK cells, enables robust activation of the effector cells to elicit an effective immune response against, for example, cancer cells. This is due to the fact that in many pathological conditions, especially cancer and viral infections, the pathogenic cells prevent an effective immune response by activating inhibitory receptors on immune cells, thereby preventing an effector response of the immune system against the pathogenic cells. However, check point inhibitors make it possible to overcome this pathological mechanism by preventing activation of the inhibitory receptors and therefore enabling and potentiating the activation of an effective immune response against the pathogenic cells.

In the context of the present invention, combined administration of the nucleic acid molecule described herein together with inhibitors of check point molecules, in particular check point molecules that are expressed by NK cells, such as for example LILRB1, inhibitory KIRs, NKG2A, PD-1, CTLA-4, TIM-3, TIGIT and LAG-3, potentiates the activating effect of the nucleic acid molecule according to the invention on NKG2C+ NK cells. Preferably, such a combined administration may be carried out in the context of the treatment of cancer or HCMV infection.

In a preferred embodiment of the present invention, the nucleic acid molecule is administered in combination with an activator of the co-stimulatory receptor CD2. It can be advantageous to use the nucleic acid molecule according to the invention in combination with an activator or stimulator of CD2, which can act as a co-stimulatory receptor on NK cells and particularly on NKG2C+ NK cells, since surprisingly the combined activation leads to an enhanced poly-functional response of the NKG2C+ NK cells including activation of cytotoxic activity as well as secretion of inflammatory mediators such as CCL3, CD107a, IFN-gamma and TNF-alpha, which cannot be explained by the addition of the individual effects of the peptides and the CD2-activators, but instead argue for the presence of a synergistic effect. Accordingly, the engagement of the co-stimulatory receptor CD2 can lower the activation threshold of NKG2C+ NK cells by the nucleic acid molecule according to the invention and therefore enable the peptides of the present invention to optimally trigger multiple effector functions in adaptive NKG2C+ NK cells.

Further embodiments of the invention relate to administration of the nucleic acid molecule in combination with IFN-alpha. IFN-alpha is known to trigger antiviral NK-cell functions and in the context of the present invention it was found that the combined administration the nucleic acid molecule according to the invention and IFN-alpha leads to an enhanced differential activation of NKG2C+ NK cells.

In embodiments of the invention, the nucleic acid molecule for use as a medicament is administered by a vector comprising the nucleic acid molecule according to the invention.

This embodiment relates to the use of the nucleic acid molecule according to the invention, which may be comprised in liposomes or other suitable formulation for administration.

For example, in embodiments a cell carrying an exogenous nucleic acid molecule comprising a nucleic acid sequence or DNA vector encoding the nucleic acid molecule of the present invention, wherein in said embodiment the nucleic acid molecule of the present invention is an RNA molecule, under the control of a constitutive or inducible promoter may be used as a vector to provide expression of the nucleic acid molecules of the invention in a subject after administration of the cells.

Similarly, a viral vector may be used to infect cells of a subject or patient in need of activation of NKG2C+ NK cells. The viral vector may comprise or encode a nucleic acid molecule according to the invention which enables expression of a peptide according to the invention in a cell of the subject upon infection with the viral vector. Alternatively, an exogenous nucleic acid molecule, such as a DNA plasmid, may be administered to a subject in need of activation of NKG2C+ NK cells, for example by means of a liposomal formulation, enabling delivery of the plasmid to a host cell of the subject, which subsequently expresses a nucleic acid molecule (e.g., RNA or DNA) of the invention or a peptide of the invention. The person skilled in the art is aware of further suitable vectors and means of administering such vectors comprising or encoding a nucleic acid molecule (e.g., RNA or DNA) or a peptide of the present invention.

In embodiments of the invention, the nucleic acid molecule according to the invention, is an RNA molecule, which is encoded by a DNA nucleic acid molecule operably linked to a promoter for expression in mammalian, preferably human subjects. In further embodiments, the nucleic acid molecule is a recombinant nucleic acid molecule. In embodiments it can be advantageous to use DNA nucleic acid molecules comprising promoters for transcription of the nucleic acid molecule or expression of a peptide of the invention in cells of the subject in need NKG2C+ NK cell activation, since it is possible to provide a source of renewed production of a peptide of the present invention in a subject with a single administration. In embodiments use of a recombinant DNA molecule is advantageous, since the transcription of the RNA molecule according to the invention can be controlled in said embodiments by a suitable promoter or promoter/enhancer combination, which is specifically selected and suitable for the specific application. In embodiments it is possible to use controllable promoters, to be able to control transcription levels of the RNA molecule according to the invention and the expression levels of peptides of the invention.

In embodiments of the invention, the vector is a genetically modified virus selected from the group comprising attenuated HCMV, vaccinia virus, adenovirus, adeno-associated virus, retrovirus, or lentivirus.

Furthermore, the invention relates in one aspect to a genetically modified virus encoding a nucleic acid molecule encoding a polypeptide of the present invention for use as a medicament to expand and/or activate NKG2C+ NK cells in the treatment and/or prevention of a medical condition associated with pathogenic cells expressing HLA-E and a peptide of the present invention.

The embodiments and associated advantages of the nucleic acid molecules of the present invention for use as a medicament also relate to the method and the cells and other embodiments of the present invention, and vice versa.

The present disclosure also includes kits, packages and multi-container units containing the described nucleic acid molecules and/or pharmaceutical compositions, active ingredients, and/or means for administering the same for use in the prevention and treatment of diseases and other conditions in mammalian subjects.

Embodiments and features of the invention described with respect to the nucleic acid molecules, the peptides, (in vitro) methods and kits are considered to be disclosed with respect to each and every other aspect and embodiment of the disclosure, such that features characterizing the nucleic acid molecule, may be employed to characterize the peptide, or kit according to the invention and vice-versa. The various aspects of the invention are unified by, benefit from, are based on and/or are linked by the common and surprising finding that nucleic acid molecules and pharmaceutical composition of the present invention are capable of inducing the activation and/or expansion of lymphocytes, such as NKG2C+ NK cells and/or T cells.

### DETAILED DESCRIPTION OF THE INVENTION

All cited documents of the patent and non-patent literature are hereby incorporated by reference in their entirety.

### Sequences:

Nucleic acid sequences and amino acid sequences of preferred nucleic acid molecules or polypeptides of the present invention are listed in Table 1.

TABLE 1
Amino acid sequences of preferred peptides encoded
by the nucleic acid molecules of the invention,
and nucleic acid sequences (cDNA) comprised by the by
nucleic acid molecules of the invention.
Amino acid (aa). The (c)DNA sequences disclosed in
the following table also comprise the corresponding
RNA sequence (having T (thymine) exchanged
for U (uracil)).

| SEQ ID | Sequence | Description |
|---|---|---|
| SEQ ID NO: 1 | VMAPRTLXL | Amino acid (aa) sequence, Therein, |
| | | X may be any amino acid, |
| | | preferably an amino acid with a |
| | | hydrophobic side chain (A, I, L, F, |
| | | V, P, G), more preferably V, L, I or |
| | | F. |
| SEQ ID NO: 2 | VMAPRTLFL | Amino acid (aa) sequence of VL9 |
| SEQ ID NO: 3 | VMAPRTLAL | Amino acid (aa) sequence |
| SEQ ID NO: 4 | VMAPRTLIL | Amino acid (aa) sequence of UL40, |
| | | aa 15-23 |
| SEQ ID NO: 5 | VMAPRTLLL | Amino acid (aa) sequence |
| SEQ ID NO: 6 | VMAPRTLVL | Amino acid (aa) sequence |
| SEQ ID NO: 7 | VMAPRTLPL | Amino acid (aa) sequence |
| SEQ ID NO: 8 | VMAPRTLGL | Amino acid (aa) sequence |
| SEQ ID NO: 9 | VMAPRTVLL | Amino acid (aa) sequence |
| SEQ ID NO: 10 | VMAPRALLL | Amino acid (aa) sequence |
| SEQ ID NO: 11 | GTGATGGCGCCGCGCACCCTGT | Nucleic acid sequence (cDNA); |
| | TTCTG | encoding the peptide sequence |
| | | VMAPRTLFL. |
| SEQ ID NO: 12 | GTGATGGCCCCTAGAACCCTGTT | Nucleic acid sequence (cDNA); |
| | CCTGTGA | encoding the peptide sequence |
| | | VMAPRTLFL. |
| SEQ ID NO: 13 | GTNATGGCNCCNMGNACNYTNT | Nucleic acid sequence (cDNA) |
| | TYYTN | encoding the peptide sequence |
| | | VMAPRTLFL; wherein N is G, A, T |
| | | or C, wherein M is A or C and |
| | | wherein Y is T or C. |
| SEQ ID NO: 14 | GTGATGGCGCCGCGCACCCTGG | Nucleic acid sequence (cDNA) |
| | CGCTG | encoding the peptide sequence |
| | | VMAPRTLAL. |
| SEQ ID NO: 15 | GTNATGGCNCCNMGNACNYTNG | Nucleic acid sequence (cDNA) |
| | CNYTN | encoding the peptide sequence |
| | | VMAPRTLAL; wherein N is G, A, T |
| | | or C, wherein M is A or C and |
| | | wherein Y is T or C. |
| SEQ ID NO: 16 | GTGATGGCGCCGCGCACCCTGA | Nucleic acid sequence (cDNA) |
| | TTCTG | encoding the peptide sequence |
| | | VMAPRTLIL. |
| SEQ ID NO: 17 | GTNATGGCNCCNMGNACNYTNA | Nucleic acid sequence (cDNA) |
| | THYTN | encoding the peptide sequence |
| | | VMAPRTLIL; wherein N is G, A, T |
| | | or C, wherein Y is T or C, wherein |
| | | M is A or C and wherein H is A, T or |
| | | C. |
| SEQ ID NO: 18 | GTGATGGCGCCGCGCACCCTGC | Nucleic acid sequence (cDNA) |
| | TGCTG | encoding the peptide sequence |
| | | VMAPRTLLL. |
| SEQ ID NO: 19 | GTNATGGCNCCNMGNACNYTNY | Nucleic acid sequence (cDNA) |
| | TNYTN | encoding the peptide sequence |
| | | VMAPRTLLL; wherein N is G, A, T |
| | | or C, wherein M is A or C and |
| | | wherein Y is T or C. |
| SEQ ID NO: 20 | GTGATGGCGCCGCGCACCCTGG | Nucleic acid sequence (cDNA) |
| | TGCTG | encoding the peptide sequence |
| | | VMAPRTLVL. |
| SEQ ID NO: 21 | GTNATGGCNCCNMGNACNYTNG | Nucleic acid sequence (cDNA) |
| | TNYTN | encoding the peptide sequence |
| | | VMAPRTLVL; wherein N is G, A, T |
| | | or C, wherein M is A or C and |
| | | wherein Y is T or C. |
| SEQ ID NO: 22 | GTGATGGCGCCGCGCACCCTGC | Nucleic acid sequence (cDNA) |
| | CGCTG | encoding the peptide sequence |
| | | VMAPRTLPL. |
| SEQ ID NO: 23 | GTNATGGCNCCNMGNACNYTNC | Nucleic acid sequence (cDNA) |
| | CNYTN | encoding the peptide sequence |
| | | VMAPRTLPL; wherein N is G, A, T |
| | | or C, wherein M is A or C and |
| | | wherein Y is T or C. |
| SEQ ID NO: 24 | GTGATGGCGCCGCGCACCCTGG | Nucleic acid sequence (cDNA) |
| | GCCTG | encoding the peptide sequence |
| | | VMAPRTLGL. |
| SEQ ID NO: 25 | GTNATGGCNCCNMGNACNYTNG | Nucleic acid sequence (cDNA) |
| | GNYTN | encoding the peptide sequence |
| | | VMAPRTLGL; wherein N is G, A, T |
| | | or C, wherein M is A or C and |
| | | wherein Y is T or C. |
| SEQ ID NO: 26 | GCCACCATGCCTAATCATCAGTC | Nucleic acid sequence (cDNA); ER- |
| | CGGGTCACCTACCGGCAGTTCA | targeting sequence is underlined; |
| | GACCTGCTCCTTGATGGCAAGAA | the VMAPRTLFL peptide encoding |
| | ACAACGAGCCCATCTGGCGCTG | region has a bold typeface, and the |
| | AGGAGAAAACGGCGACGGGAAA | stabilizing sequence is shown in |
| | TGCGCAAGATTAACCGAAAGGT | italic typeface. |
| | GAGAAGAATGAATCTCGCACCCA | |
| | TTAAAGAAAAAACAGCCTGGCAG | |
| | CACCTGCAAGCTCTGATCTTCGA | |
| | GGCGGAAGAAGTGTTGAAGACT | |
| | TCTCAAACTCCACAGACCTCCCT | |
| | CACGCTGTTTCTAGCACTCTTGG | |
| | CCGTATTGGCCCCGCCACCTGT | |
| | GAGCGGGGTGATGGCCCCTAGA | |
| | ACCCTGTTCCTGTGAAGCTCGCT | |
| | TTCTTGCTGTCCAATTTCTATTAA | |
| | AGGTTCCTTTGTTCCCTAAGTCC | |
| | AACTACTAAACTGGGGGATATTA | |
| | TGAAGGGCCTTGAGCATCTGGA | |
| | TTCTGCCTAATAAAAAACATTTAT | |
| | TTTCATTGCAGCTCGCTTTCTTG | |
| | CTGTCCAATTTCTATTAAAGGTTC | |
| | CTTTGTTCCCTAAGTCCAACTAC | |
| | TAAACTGGGGGATATTATGAAGG | |
| | GCCTTGAGCATCTGGATTCTGCC | |
| | TAATAAAAAACATTTATTTTCATT | |
| | GC | |
| SEQ ID NO: 27 | X₁X₂PX₃RSLX₄X₅ | Amino acid (aa) sequence, therein |
| | | each X is an amino acid, wherein P |
| | | is proline, R is Arginine, S is serine, |
| | | L is Leucine, X₁ is Threonine or |
| | | Valine, X₂ is Glycine or Asparagine, |
| | | X₃ is Tryptophan or Glycine, X₄ is |
| | | Tryptophan or Phenylalanine, and |
| | | X₅ is Leucine or Isoleucine. |
| SEQ ID NO: 28 | X₁X₆PX₇RX₈X₉FL | Amino acid (aa) sequence, therein |
| | | each X is an amino acid, wherein P |
| | | is proline, R is Arginine, F is |
| | | Phenylalanine, L is Leucine, X₁ is |
| | | Threonine or Valine, X₆ is Alanine |
| | | or Asparagine, X₇ is Alanine or |
| | | Glycine, X₈ is Serine or Threonine, |
| | | and X₉ is Leucine or Methionine. |
| SEQ ID NO: 29 | X₁₀X₁₁X₁₂X₁₃RX₁₄X₁₅X₁₆X₁₇, | Amino acid (aa) sequence, therein |
| | | X₁₀ is Threonine, Isoleucine, |
| | | Phenylalanine, Asparagine, |
| | | Arginine, Glutamine or Valine, X₁₁ is |
| | | Glycine, Asparagine, Alanine, |
| | | Leucine or Methionine, X₁₂ is |
| | | Threonine or Proline, X₁₃ is Alanine, |
| | | Tryptophan, Glycine, Proline, |
| | | Histidine, Asparagine, Aspartic acid, |
| | | Glutamic acid, Lysine or Serine, R |
| | | is Arginine, X₁₄ is Serine, Glycine or |
| | | Threonine, X₁₅ is Leucine, Valine, |
| | | Glutamine or Methionine, X₁₆ is |
| | | Tryptophan, Tyrosine, Alanine, |
| | | Isoleucine, Leucine, Phenylalanine, |
| | | Valine, Proline, Cysteine or Glycine, |
| | | and X₁₇ is Leucine, Phenylalanine |
| | | or Isoleucine. |
| SEQ ID NO: 30 | QMPSRSLLF | aa sequence of CREB3L1, aa 419- |
| | | 427 |
| SEQ ID NO: 31 | TLPKRGLFL | aa sequence of AKAP6, aa 388-396 |
| SEQ ID NO: 32 | TGPWRSLWI | aa sequence of GTF3C5, aa 293- |
| | | 301 |
| SEQ ID NO: 33 | ILTDRSLWL | aa sequence of FBXO41, aa 670- |
| | | 678 |
| SEQ ID NO: 34 | VNPGRSLFL | aa sequence of BFAR, aa 263-271 |
| SEQ ID NO: 35 | WNRLFPPLR | aa sequence of UBAC2, aa 275- |
| | | 279 |
| SEQ ID NO: 36 | TLPERTLYL | aa sequence of ECEL1, aa 269-277 |
| SEQ ID NO: 37 | FLPNRSLLF | aa sequence of SLC52A3, aa 354- |
| | | 362 |
| SEQ ID NO: 38 | VMGDRSVLY | aa sequence of EMC1, aa 725-733 |
| SEQ ID NO: 39 | VMADKSIFY | aa sequence of OR5D14, aa 88-96 |
| SEQ ID NO: 40 | VMPPRTLLL | aa sequence of HLA-A, aa 3-11 |
| SEQ ID NO: 41 | VMPGRTLCF | aa sequence of TACR3, aa 226-234 |
| SEQ ID NO: 42 | RMPPRSVLL | aa sequence of INTS1, aa 260-268 |
| SEQ ID NO: 43 | TAPARTMFL | aa sequence of PISD, aa 55-63 |
| SEQ ID NO: 44 | NMPARTVLF | aa sequence of MTREX, aa 490- |
| | | 498 |
| SEQ ID NO: 45 | VLPHRTQFL | aa sequence of UL120, aa 72-80, |
| | | Merlin |
| SEQ ID NO: 46 | LTDRSLWL | aa sequence of FBXO41, aa 670- |
| | | 678 |
| SEQ ID NO: 47 | ATGCCTAATCATCAGTCCGGGTC | Nucleic acid sequence of a gp70 |
| | ACCTACCGGCAGTTCAGACCTG | ER-targeting sequence of a mouse |
| | CTCCTTGATGGCAAGAAACAACG | mammary tumor virus (MMTV) |
| | AGCCCATCTGGCGCTGAGGAGA | envelope gene. |
| | AAACGGCGACGGGAAATGCGCA | |
| | AGATTAACCGAAAGGTGAGAAGA | |
| | ATGAATCTCGCACCCATTAAAGA | |
| | AAAAACAGCCTGGCAGCACCTG | |
| | CAAGCTCTGATCTTCGAGGCGG | |
| | AAGAAGTGTTGAAGACTTCTCAA | |
| | ACTCCACAGACCTCCCTCACGCT | |
| | GTTTCTAGCACTCTTGGCCGTAT | |
| | TGGCCCCGCCACCTGTGAGCGG | |
| | G | |
| SEQ ID NO: 48 | MPNHQSGSPTGSSDLLLDGKKQR | aa sequence of a gp70 ER- |
| | AHLALRRKRRREMRKINRKVRRM | targeting sequence of MMTV |
| | NLAPIKEKTAWQHLQALIFEAEEVL | (modified/engineered) |
| | KTSQTPQTSLTLFLALLAVLAPPPV | |
| | SG | |
| SEQ ID NO: 49 | AGCTCGCTTTCTTGCTGTCCAAT | Nucleic acid sequence of a beta- |
| | TTCTATTAAAGGTTCCTTTGTTCC | globin-derived stability inducing |
| | CTAAGTCCAACTACTAAACTGGG | sequence |
| | GGATATTATGAAGGGCCTTGAGC | |
| | ATCTGGATTCTGCCTAATAAAAA | |
| | ACATTTATTTTCATTGCAGCTCG | |
| | CTTTCTTGCTGTCCAATTTCTATT | |
| | AAAGGTTCCTTTGTTCCCTAAGT | |
| | CCAACTACTAAACTGGGGGATAT | |
| | TATGAAGGGCCTTGAGCATCTG | |
| | GATTCTGCCTAATAAAAAACATTT | |
| | ATTTTCATTGC | |
| SEQ ID NO: 50 | TTAAAGGTTCCTTTGTTCCCTAA | Nucleic acid sequence of a beta- |
| | GTCCAACTACTAAACTGGGGGAT | globin-derived stability inducing |
| | ATTATGAAGGGCCTTGAG | sequence |
| SEQ ID NO: 51 | TTCCTTTGTTCCCT | Nucleic acid sequence of a beta- |
| | | globin-derived stability inducing |
| | | sequence |
| SEQ ID NO: 52 | GGGGGATATTAT | Nucleic acid sequence of a beta- |
| | | globin-derived stability inducing |
| | | sequence |
| SEQ ID NO 53 | VMAPQSLLL | “PQS”, negative control peptide |

In embodiments, the peptide encoded by the nucleic acid sequence comprised within the nucleic acid molecule of the invention has a length of 5-30 amino acids. In embodiments the peptide encoded by the nucleic acid sequence has a length, for example, of 5-25, 5-20, 5-15, 5-10, 8-30, 8-25, 8-20, 8-15, 8-10, 9-30, 9-29, 9-28, 9-27, 9-26, 9-25, 9-24, 9-23, 9-22, 9-21, 9-20, 9-19, 9-18, 9-17, 9-16, 9-15, 9-14, 9-13, 9-12, 9-11, or 9-10 amino acids. The length of the peptide may be defined by a range formed by any of the above values, for example any one value in combination with any other value, as end points of the range.

In embodiments the expressed or encoded polypeptide has a length of at least 7, 8, 9, 10, 11, 12, 13, 14, 15, 16, 17, 18, 19, 20, 21, 22, 23, 24, 25, 26, 27, 28, 29 or 30 amino acids, preferably of 9 amino acids (aa), optionally +1-10 aa. In embodiments the expressed or encoded polypeptide has a length of between 7 and 30 amino acids, preferably of between 7 and 30 aa, or 9 and 30 aa. In embodiments the encoded polypeptide has a length of less than 100, 90, 80, 70, 60, 50, 40 or 30 aa, preferably of less than 30, 20, 10 or 9 amino acids.

In one embodiment the nucleic acid molecules according to the invention encode a polypeptide as described herein, comprising or consisting of an amino acid sequence selected from the group consisting of:
a) an amino acid sequence comprising or consisting of an amino acid sequence according to any one of SEQ ID NO 1-10 or 27-46; wherein the encoded polypeptide is preferably no longer than 100, 90, 80, 70, 60, 50 or 40, preferably 30, more preferably 20, most preferably no longer than 10 or 9 amino acids;
b) an amino acid sequence comprising or consisting of an amino acid sequence according to any one of SEQ ID NO 1-10 or 27-46, wherein the length of the encoded polypeptide is between 5 and 300 amino acids, 6 and 200 amino acids, 7 and 100, 8 and 50, preferably between 9 and 30 amino acids, wherein the surrounding encoded amino acid sequences are preferably provided as UL-40 sequences flanking the encoded amino acid sequences according to any one of SEQ ID NO 1-10 or 27-46, or as sequences from MHC class I molecules, preferably non-classical MHC class I molecules, most preferably HLA-G, preferably flanking the encoded signal peptide (also called leader sequence or leader peptide or signal sequence) of the respective MHC class I molecule,
c) an amino acid sequence having sufficient sequence identity to be functionally analogous/equivalent to an amino acid sequence according to a), comprising preferably a sequence identity to an amino acid sequence according to a) of at least 70%, 80%, preferably 90%, more preferably 95%; and
d) an amino acid sequence of a), b) or c) which is modified by deletions, additions, substitutions, translocations, inversions and/or insertions and functionally analogous/equivalent to an amino acid sequence according to a), b) or c).

Functionally analogous sequences refer preferably to the ability to induce to induce expansion and/or activation of NKG2C+ natural killer (NK) cells.

Embodiments of the invention may comprise nucleic acid molecules comprising a nucleic acid sequence encoding a polypeptide as described herein comprising or consisting of an amino acid sequence SEQ ID NO 1-10 or 27-46, or variants of these sequences, wherein the sequence variant may comprise a sequence identity to SEQ ID NO 1-10 or 27-46 of 50, 55, 60, 65, 70, 75, 80, 81, 82, 83, 84, 85, 86, 87, 88, 89, 90, 91, 92, 93, 94, 95, 96, 97, 98 or 99%. Sequence identity may be determined using methods known to one skilled in the art, such as BLAST or other sequence alignment tools.

In some embodiments of the invention the peptide, preferably according to sequences disclosed herein, may comprise a 0 to 10 amino acid addition or deletion at the N and/or C terminus of a sequence.

As used herein the term “a 0 to 10 amino acid addition or deletion at the N and/or C terminus of a sequence” means that the polypeptide may have a) 0, 1, 2, 3, 4, 5, 6, 7, 8, 9 or 10 additional amino acids at its N terminus and 0, 1, 2, 3, 4, 5, 6, 7, 8, 9 or 10 amino acids deleted at its C terminus or b) 0, 1, 2, 3, 4, 5, 6, 7, 8, 9 or 10 additional amino acids at its C terminus and 0, 1, 2, 3, 4, 5, 6, 7, 8, 9 or 10 nucleotides deleted at its N terminus, c) 0, 1, 2, 3, 4, 5, 6, 7, 8, 9 or 10 additional amino acids at its N terminus and 0, 1, 2, 3, 4, 5, 6, 7, 8, 9 or 10 additional amino acids at its N terminus or d) 0, 1, 2, 3, 4, 5, 6, 7, 8, 9 or 10 amino acids deleted at its N terminus and 0, 1, 2, 3, 4, 5, 6, 7, 8, 9 or 10 amino acids deleted at its C terminus.

In further preferred embodiments, the invention relates to a nucleic acid molecule comprising a nucleic acid sequence encoding a polypeptide comprising or consisting of an amino acid sequence derived from the UL-40 protein of HCMV.

In further embodiments, the invention relates to a nucleic acid molecule comprising a nucleic acid sequence encoding a polypeptide comprising or consisting of an amino acid sequence derived from the signal sequence of a MHC class I molecule, preferably a non-classical MHC class I molecules, most preferably HLA-E.

Preferably, the amino acid sequence of the encoded peptide has a length of at least 7 amino acids, more preferably 8 amino acids, most preferably 9 amino acids. In some embodiments, the peptide may comprise 10 or more amino acids.

“Sequence variants” or “variants” of proteins or peptides, as defined in the context of the present invention, may be generated that have an amino acid sequence that differs from an original sequence in one or more mutation(s), such as one or more substituted, inserted and/or deleted amino acid(s). In embodiments, the sequence variation described here with respect to conservative substitutions and/or percentage identity, may apply to any one or more embodiments, described throughout the application as a whole.

Preferably, these fragments and/or variants have the same biological function or specific activity compared to the native full length protein, e.g. its specific antigenic property. “Variants” of proteins or peptides, as defined in the context of the present invention, may comprise one or more conservative amino acid substitution(s) compared to their native, i.e. non-mutated, physiological sequence. These amino acid sequences, as well as their coding nucleotide sequences, more particularly fall within the term “variants” as defined herein. Substitutions in which amino acids originating from the same class are exchanged for each other are called conservative substitutions. These are in particular amino acids with aliphatic side chains, positively or negatively charged side chains, aromatic groups in the side chains or amino acids whose side chains can form hydrogen bonds, e.g., side chains that have a hydroxyl function. This means that, for example, an amino acid with a polar side chain is replaced by another amino acid with a side chain that is also polar, or, for example, an amino acid characterised by a hydrophobic side chain is replaced by another amino acid with a side chain that is also hydrophobic (e.g., serine (threonine) by threonine (serine) or leucine (isoleucine) by isoleucine (leucine)).

Insertions and substitutions are possible especially at such sequence positions that do not cause a change in the three-dimensional structure or do not affect the binding region. Modifications to a three-dimensional structure by insertion(s) or deletion(s) can be easily determined, e.g., using circular dichroism spectra (CD spectra) (Urry, 1985, Absorption, Circular Dichroism and ORD of Polypeptides, in: Modern Physical Methods in Biochemistry, Neuberger et al., (eds.), Elsevier, Amsterdam).

Furthermore, variants of proteins or peptides as defined herein that may be encoded by a nucleic acid molecule may also comprise such sequences, wherein nucleotides of the encoding nucleic acid sequence are exchanged according to the degeneracy of the genetic code without any change in the respective amino acid sequence of the protein or peptide, i.e. the amino acid sequence or at least a part thereof may not differ from the original sequence in one or more mutation(s) as defined above.

Protein modifications to the polypeptides of the present invention, which may occur through substitutions in amino acid sequence, and nucleic acid sequences encoding such molecules, are also included within the scope of the invention.

Nucleic acid substitutions as defined herein are modifications made to the nucleic acid sequence of the nucleic acid molecule, whereby one or more nucleic acids are replaced with the same number of (different) nucleic acids, such that the nucleic acid sequence is changed and a protein is encoded, which, in certain embodiments, contains a different amino acid sequence than the primarily protein encoded by the primary nucleic acid. In some embodiments this amendment will not alter the encoded amino acid sequence of the protein. Like additions, substitutions may be natural or artificial. It is well known in the art that nucleic acid substitutions may be made without altering the encoded amino acid sequence of the encoded protein and/or the encoded proteins function.

Amino acid substitutions as defined herein are modifications made to the amino acid sequence of the protein, whereby one or more amino acids are replaced with the same number of (different) amino acids, producing a protein which contains a different amino acid sequence than the primary protein. In some embodiments this amendment will not significantly alter the function of the protein. Like additions, substitutions may be natural or artificial. It is well known in the art that amino acid substitutions may be made without significantly altering the protein's function. This is particularly true when the modification relates to a “conservative” amino acid substitution, which is the substitution of one amino acid for another of similar properties. Such “conserved” amino acids can be natural or synthetic amino acids which because of size, charge, polarity and conformation can be substituted without significantly affecting the structure and function of the protein. Frequently, many amino acids may be substituted by conservative amino acids without deleteriously affecting the protein's function.

In general abbreviations used for amino acids herein are the following: Alanine is Ala or A, Arginine is Arg or R, Asparagine is Asn or N, Aspartic acid is Asp or D, Cysteine is Cys or C, Glutamic acid is Glu or E, Glutamine is Gln or Q, Glycine is Gly or G, Histidine is His or H, Isoleucine is Ile or I, Leucine is Leu or L, Lysine is Lys or K, Methionine is Met or M, Phenylalanine is Phe or F, Proline is Pro or P, Serine is Ser or S, Threonine is Thr or T, Tryptophan is Trp or W, Tyrosine is Tyr or Y and Valine is Val or V. J is Leucine or Isoleucine. In embodiments herein the terms peptide and polypeptide may be used interchangeably. Also, the terms protein and polypeptide may be used herein interchangeably.

In general, the non-polar amino acids Gly, Ala, Val, Ile and Leu; the non-polar aromatic amino acids Phe, Trp and Tyr; the neutral polar amino acids Ser, Thr, Cys, Gln, Asn and Met; the positively charged amino acids Lys, Arg and His; the negatively charged amino acids Asp and Glu, represent groups of conservative amino acids. This list is not exhaustive. For example, it is well known that Ala, Gly, Ser and sometimes Cys can substitute for each other even though they belong to different groups.

A hydrophobic side-chain of an amino acid commonly refers to the chemical side chain of an amino acid which does not like to reside in an aqueous, e.g., water, environment. Hydrophobic side chains are considered to be comprised in the amino acids alanine (Ala), valine (Val), leucine (Leu), isoleucine (Ile), phenylalanine (Phe), methionine (Met), Tyrosine (Tyr) and tryptophan (Trp).

### ER-Targeting Sequence, Cleavage Sites, Stability Inducing Motif, Cap Structure;

As used herein, an endoplasmic reticulum (ER)-targeting sequence refers to a sequence or signal sequence that enables the transport or transition of a nucleic acid molecule or peptide, in which said ER-targeting sequence is comprised, to the endoplasmic reticulum (ER). In the art a signal peptide (also termed signal sequence or targeting sequence) is a short peptide (usually <35 aa) present mostly at the N-terminus (occasionally at the C-terminus or internally) of most newly synthesized proteins destined for the secretory pathway (e.g., through the ER). The skilled person knows how to identify suitable ER-targeting sequences, e.g., at http://www.signalpeptide.de or using state-of-the-art models to predict signal peptides and their cleavage sites (e.g., according to 10.1038/s41587-021-01156-3⁶⁸). In some embodiments, the ER-targeting sequence may comprise or constitute of any suitable ER signal sequence. Herein, the ER-targeting sequence is in certain preferred embodiments the mouse mammary tumor virus (MMTV) envelope glycoprotein gp70. In embodiments, the nucleic acid molecule comprises a nucleic acid sequence encoding an ER-targeting or signaling sequence of a mouse mammary tumor virus (MMTV; e.g., SEQ ID NOs: 26 and 48-49).

In some embodiments the nucleic acid molecule may comprise one or more enzyme cleavage sites, such as e.g., a cleavage site for an ER-associated peptidase. The cleavage of the polypeptide, which is encoded by the nucleic acid molecule, by an ER-associated peptidase enables the ER-targeting sequence to be cleaved from the encoded peptide, e.g., such that the encoded peptide can be loaded onto HLA-E. The skilled person knows how to select suitable cleavage sites, e.g., cleavage sites for an ER-associated peptidase, for example, at http://www.signalpeptide.de, or as described by von Heijne (von Heijne; Patterns of amino acids near signal-sequence cleavage sites. 1983; Eur J Biochem 133 (1) 17-21), or using state-of-the-art models to predict signal peptides and their cleavage sites (e.g., according to 10.1038/s41587-021-01156-3⁶⁸).

Assembly of MHC class I molecules (e.g., human MHC class Ib molecule HLA-E) commonly occurs in the endoplasmic reticulum (ER). HLA-E has been shown to be able to bind signal-sequence-derived peptides, wherein the preferred peptide is a nonamer.

The terms signal peptide, leader peptide, leader sequence and signal sequence are used interchangeably in the context of the present invention and refer to is a short peptide of up to around 30 amino acids length present at the N-terminus of the majority of newly synthesized proteins that are destined towards the secretory pathway. These proteins include those that reside either inside certain organelles (the endoplasmic reticulum, golgi or endosomes), secreted from the cell, or inserted into most cellular membranes. In particular, the terms may be used herein when referring to the signal peptides comprised by classical and non-classical MHC class I molecules.

A stability inducing motif herein may refer to any nucleic acid motive suitable to increase the stabilization of a nucleic acid molecule according to the invention. In embodiments, wherein the nucleic acid molecule is an RNA molecule, the stability of the nucleic acid molecule, may be increased by inserting a stability inducing motif at the 3′UTR of the RNA molecule, e.g., at the 3′UTR stem-loop structure of the RNA molecule. Said stability inducing motif can constitute a sequence of a naturally, e.g., in mammalians or humans, occurring sequence or a modified or artificial nucleic acid sequence.

A cap structure or 5′-cap structure or simple ‘cap’ is a cap-like attachment at the 5′-end of RNA molecules, commonly mRNA molecules, which is, in nature, attached in the nucleus of eukaryotic cells. A RNA-cap structure may also be attached to an RNA molecule under artificial and/or vitro conditions. The 5′-cap commonly not only protects an RNA from degradation but can also be important for the export of an (m) RNA from a cells' nucleus into the cytoplasm and for the translation of the mRNA by ribosomes. The 5′-cap structure is in eukaryotes often a modified guanine nucleotide that is attached to the head end of the RNA via a rare 5′-5′-phosphodiester bond. In nature the chemical capping reaction takes place during the transcription of a gene as soon as an RNA polymerase has linked the first nucleotides of an mRNA. In contrast to the (co-transcriptional) capping of the 5′-end, the polyadenylation of the 3′-end of an mRNA—called tailing—is in nature a post-transcriptional modification that is only carried out after the separation of mRNA and RNA polymerase.

Commonly a three prime untranslated region (3′-UTR) is a section of an RNA molecule, such as a messenger RNA (mRNA), that immediately follows the translation termination codon (“stop codon”). Nucleic acid sequences within the 3′-UTR may in embodiments have the ability to regulate the stability of the RNA molecule, herein preferably stabilize the RNA molecule, which can influence the expression of an encoded peptide.

### NK Cells:

In embodiments of the invention, the nucleic acid molecule is used as a medicament to expand and/or activate NKG2C+ natural killer (NK) cells in the treatment and/or prevention of a medical condition associated with pathogenic cells expressing HLA-E and a peptide comprising an amino acid sequence according to SEQ ID NO 1-10 or 27-46.

Natural killer cells (NK cells) are cytotoxic lymphocytes of the innate immune system. NK cells provide rapid responses to viral-infected cells, acting at around 3 days after infection, and respond to tumor formation. Typically, immune cells detect major histocompatibility complex (MHC) presented on infected cell surfaces, triggering cytokine release, causing lysis or apoptosis. NK cells are unique, however, as they have the ability to recognize stressed cells in the absence of antibodies and MHC, allowing for a much faster immune reaction. They were named “natural killers” because of the initial notion that they do not require activation to kill cells that are missing “self” markers of MHC class 1. This role is especially important because harmful cells that are missing MHC I markers cannot be detected and destroyed by other immune cells, such as T lymphocyte cells.

CD94/NKG2 is a family of C-type lectin receptors which are expressed predominantly on the surface of NK cells. These receptors stimulate or inhibit cytotoxic activity of NK cells, therefore they are divided into activating and inhibitory receptors according to their function. CD94/NKG2 recognize non-classical MHC glycoproteins class I. CD94/NKG2 family includes seven members: NKG2A, B, C, D, E, F and H. NKG2 receptors are transmembrane proteins type II which dimerize with CD94 molecule. CD94 contains a short cytoplasmic domain and it is responsible for signal transduction. Therefore, NKG2 receptors form disulfide bonded heterodimers with CD94. NKG2D represent an exception, since it predominantly forms a homodimer.

NKG2A and NKG2B receptors transmit inhibitory signal. They contain two immuno-receptor tyrosine-based inhibitory motives (ITIM) in their cytoplasmic tail, which transduces the signal upon engagement of a ligand through Src family kinases, and the tyrosine phosphatase SHP-1, SHP-2 or SHIP. As a result, NK cell activation is suppressed. The CD94/NKG2A-dimer is an HLA-E receptor heterodimer and is expressed by NK cells and certain T cells.

NKG2C, NKG2E and NKG2H are activating receptors. Ligand binding enables interaction between receptor and the ITAM-bearing adaptor protein DAP12. Subsequent signaling through Src family kinases, the tyrosine kinases Syk and ZAP-70 can lead to release cytolytic granules containing perforin and granzyme and production of many cytokines and chemokines. NKG2D is activating receptor as well but it couples with adaptor protein DAP10 and triggers actin reorganization (cell polarization) and degranulation upon ligand engagement. The function of NKG2F receptor is not clear. The CD94/NKG2C-dimer is an HLA-E receptor heterodimer and is expressed by NK cells and certain T cells.

Receptors of CD94/NKG2 family bind non-classical MHC glycoproteins class I. Non-classical MHC glycoproteins class I are structurally similar to classical MHC class I molecules, but they present mainly peptides derived from the signal peptides of MHC class I. Therefore, NK cells can indirectly monitor the expression of classical MHC class I molecules through the interaction of CD94/NKG2 with HLA-E. The binding of CD94/NKG2A and CD94/NKG2C to HLA-E is dependent on the presented peptides, and NK cells integrate signals from these and other receptors to modulate their cytotoxic activity against said HLA-E presenting cells.

Non-classical MHC class I molecules comprise HLA-G, HLA-E and HLA-F. For HLA-G, 7 protein isoforms have been described. Four of these isoforms are membrane-bound (HLA-G 1-4) while 3 of them lack exons 5-7, hence and existing as secreted forms (HLA-G 5-7). Of all membrane-bound HLA-G variants, HLA-G1 represents the sole full-length version of the molecule. Conversely, HLA-G2 does not contain exon 3, HLA-G3 is missing exons 3 and 4, and HLA-G4 does not include exon 4. The soluble isoforms of HLA-G (namely, HLA-G5, HLA-G6, and HLA-G7) contain part of intron 4, harboring a stop codon. This results in the expression of truncated proteins lacking exon 5, which encodes the transmembrane domain. HLA-G5, -G6, and -G7 represent the soluble counterparts of HLA-G1, G2, and -G3, respectively. HLA-E consists of 8 exons, wherein the first encodes the leader peptide sequence, exons 2, 3 and 4 encode the MHC immunoglobulin-like a domains 1, 2, and 3, respectively, exon 5 encodes the transmembrane domain and exons 6 and 7 encode the cytoplasmic tail. Similar to HLA-G, HLA-E forms a complex with β2 microglobulin. HLA-E consists of 8 exons. Of these, the first encodes the leader peptide sequence, exons 2, 3 and 4 encode the MHC immunoglobulin-like a domains 1, 2, and 3, respectively, exon 5 encodes the transmembrane domain and exons 6 and 7 encode the cytoplasmic tail. Similar to HLA-G, HLA-E forms a complex with β2 microglobulin.

HLA-G, -E, and -F are important regulators of the immune system and the upregulation of HLA-G, -E, and -F following IFNγ stimulation suggests that non-classical MHC class I molecules may be involved in negative feedback responses to potentially harmful pro-inflammatory responses. While inflammatory responses are required to eliminate cancer cells, they also trigger strong immunoregulatory mechanisms that limit the recognition of malignant cells by the immune system, hence favoring tumor progression. Non-classical MHC class I molecules constitute means whereby malignant cells escape immunosurveillance. Indeed, these molecules inhibit the activity of the immune system by binding to inhibitory receptors expressed by effector cells, hence suppressing their functions or inducing their apoptotic demise (Kochan et al. Oncoimmunology. 2013 Nov. 1; 2(11): e26491; Smyth et al. Oncoimmunology. 2013 Mar. 1; 2(3): e23336).

HLA-E has a very specialized role in cell recognition by NK cells by binding a restricted subset of peptides derived from signal peptides of classical and non-classical MHC class I molecules, such as HLA-A, B, C, G. These peptides are released from the membrane of the endoplasmic reticulum (ER) by the signal peptide peptidase, trimmed by the cytosolic proteasome, transport into the ER lumen by the transporter associated with antigen processing (TAP) and subsequently bound to the groove on the HLA-E molecule. This allows HLA-E to assemble correctly and to be stabilized, leading to expression on the cell surface. NK cells recognize the complex formed by HLA-E+ peptide using the heterodimeric inhibitory receptors CD94/NKG2A, B and/or C. When CD94/NKG2A or CD94/NKG2B is engaged, it produces an inhibitory effect on the cytotoxic activity of the NK cell to prevent cell lysis, whereas binding of HLA-E to CD94/NKG2C results in NK cell activation. This interaction has been shown to trigger expansion of NK cell subsets in antiviral responses.

HLA-G may play a role in immune tolerance in pregnancy, being expressed in the placenta by extravillous trophoblast cells (EVT), while the classical MHC class I genes (HLA-A and HLA-B) are not expressed. HLA-G is a ligand for NK cell inhibitory receptor KIR2DL4, and therefore expression of this HLA by the trophoblast defends it against NK cell-mediated death. Aberrant induction of HLA-G expression has been observed in various malignancies and strongly associated with tumor immune escape, metastasis and poor prognosis. HLA-G, membrane-bound or soluble, strongly binds its inhibitory receptors on immune cells (NK, T, B, monocytes/dendritic cells), inhibits the functions of these effectors, and so induces immune inhibition. HLA-G function may therefore be beneficial and protective when expressed by a fetus or a transplant, but deleterious when expressed by a tumor or cancer cells, because it also protects malignant cells from antitumor immunity. Accordingly, HLA-G can be classified as a checkpoint molecule.

As used herein, expansion and/or activation of NKG2C+ NK cells refers, in embodiments, to the stimulation of NKG2C+ NK cells with an activating signal leading to the execution of effector functions, such as release of cytotoxic granules and production of pro-inflammatory cytokines and chemokines, and/or the induction of survival and/or proliferation of the cells.

### Medical Conditions:

As used herein, “treatment” of a disease or “treating” a subject afflicted with a disorder shall mean slowing, stopping or reversing the disorder's progression. In the preferred embodiment, treating a subject afflicted with a disorder means reversing the disorder's progression, ideally to the point of eliminating the disorder itself. As used herein, ameliorating a disorder and treating a disorder are equivalent. The treatment of the present invention may also, or alternatively, relate to a prophylactic administration of the active agents described herein. Such a prophylactic administration may relate to the prevention of any given medical disorder, or the prevention of development of said disorder, whereby prevention or prophylaxis is not to be construed narrowly under all conditions as absolute prevention. Prevention or prophylaxis may also relate to a reduction of the risk of a subject developing any given medical condition, preferably in a subject at risk of said condition.

The term “medical conditions associated with pathogenic cells expressing HLA-E and a peptide comprising an amino acid sequence according to SEQ ID NO 1-10 or 27-46” relates to several pathologies that share the common feature of the presence of pathological cells expressing HLA-E as well as a peptide of the present invention, wherein these cells are involved in the pathological mechanisms.

Such pathologies include, for example, the situation of active HCMV infection, which may be a new infection or a reactivation of a latent infection, wherein the UL-40 protein of HCMV is expressed in a host cell that expresses HLA-E. Furthermore, such pathologies include certain cancers, wherein the cancer cells express HLA-G. The signal sequence of HLA-G comprises an amino acid sequence corresponding to the peptide of the present invention, and the signal sequence gets processed inside the cancer as described above. Cancers expressing both, HLA-E and HLA-G, have been described in the art for melanoma, choriocarcinoma, breast cancer, endometrial cancer, ovarian cancer, cervical cancer, esophageal squamous cell carcinoma, colorectal cancer, gastric cancer, hepatocellular carcinoma, glioblastoma, lung cancer, nasopharyngeal carcinoma, pancreatic adenocarcinoma, thyroid carcinoma and renal carcinoma (Curigliano G, Criscitiello C, Gelao L, Goldhirsch A. Molecular pathways: human leukocyte antigen G (HLA-G). Clin Cancer Res. 2013; 19(20):5564-71; Lin A, Yan W H. HLA-G expression in cancers: roles in immune evasion, metastasis and target for therapy. Mol Med. 2015; Seliger B, Schlaf G. Structure, expression and function of HLA-G in renal cell carcinoma. Semin Cancer Biol. 2007; 17(6):444-50), and methods and techniques for determining the expression of HLA-E and HLA-G in a pathological cell are known to the skilled person.

Medical conditions and cancers associated with pathogenic cells expressing HLA-E and a peptide comprising an amino acid sequence according to SEQ ID NO 1-10 or 27-46, that are treatable by the effector function of said NKG2C+ NK cells, and/or that are susceptible to NKG2C+ NK cell cytotoxic activity comprise, without limitation, melanoma, choriocarcinoma, breast cancer, endometrial cancer, ovarian cancer, cervical cancer, esophageal squamous cell carcinoma, colorectal cancer, gastric cancer, hepatocellular carcinoma, glioblastoma, lung cancer, nasopharyngeal carcinoma, pancreatic adenocarcinoma, thyroid carcinoma and renal carcinoma, and in particular cancer types previously described to be susceptible to immunotherapy, such as melanoma, renal cell carcinoma and hematological malignancies.

The invention is based on the surprising finding that the nucleic acid molecules of the present invention can induce expansion and activation of NKG2C+ NK cells in vivo and in vitro. Accordingly, the nucleic acid molecules can be used as a medicament in the treatment of medical condition treatable by the effector function of said NKG2C+ NK cells. The receptor complex of CD94/NKG2C is an activating receptor of NKG2C+ NK cells and accordingly, the cells are useful in the treatment of diseases that are associated with pathological cells expressing the ligand of this receptor on their surface, such as the complex of HLA-E with the bound peptide of the present invention.

In addition to cancer cells, pathogenic cells expressing HLA-E and peptides resembling SEQ ID NO 1-10 or 27-46, could plausibly represent immune effector cells exacerbating pathology during inflammatory disorders, such as but not limited to rheumatic diseases and other autoimmune conditions.

In embodiments of the present invention, the peptide is used as a medicament to inhibit reactivation of human cytomegalovirus (HCMV) infections and/or reduce viral titers in an individual infected with HCMV.

Human cytomegalovirus (HCMV) is a species of the Cytomegalovirus genus of viruses, which in turn is a member of the viral family known as Herpesviridae or herpesviruses. It is typically abbreviated as HCMV or, commonly but more ambiguously, as CMV. It is also known as human herpesvirus-5 (HHV-5). HCMV infection is typically unnoticed in healthy people, but can be life-threatening for the immune-compromised, such as HIV-infected persons, organ transplant recipients, or newborn infants. Congenital cytomegalovirus infection can lead to significant morbidity and even death. After infection, HCMV remains latent within the body throughout life and can be reactivated at any time. Eventually, it may cause mucoepidermoid carcinoma and possibly other malignancies such as prostate cancer.

UL-40 is protein of 221 amino acids of HCMV, which plays a role in viral immune evasion. Human CMV has evolved multiple strategies to interfere with immune recognition of the host. A variety of mechanisms target Ag presentation by MHC class I molecules resulting in a reduced class I cell-surface expression. This down-regulation of class I molecules can trigger NK cytotoxicity, which would have to be counteracted by the virus to establish long-term infection. The UL-40 protein of HCMV, which is encoded by the open reading frame UL-40, encodes a canonical ligand for HLA-E, and expression of UL-40 in HLA-E-positive target cells is thought to induce resistance to NK cell lysis via the CD94/NKG2A receptor. However, as disclosed herein, UL-40 can comprise the peptides of the present invention and therefore HCMV infected cells may express the complex of HLA-E and the peptide of the present invention on their surface. Accordingly, such cells can be recognized by CD94/NKG2C, which leads to activation of NKG2C+ NK cells.

Accordingly, the present invention can be used to inhibit reactivation of human cytomegalovirus (HCMV) infections and/or reduce viral titers in an individual infected with HCMV. By means of the present invention, the number of NKG2C+ NK cells in a host can be increased, either by administration of the nucleic acid molecules of the present invention or by administering in vitro expanded NKG2C+ NK cells of the present invention. The invention can be applied to patients that are newly infected with HCMV and suffer from an active infection to reduce the viral load and to stop or accelerate the containment of the active infection. Furthermore, the invention can be applied in the context of a reactivation of HCMV infection in a latently infected individual. Additionally, the invention can be applied to prevent clinical reactivation in individuals at risk, for example because they are latently infected or receive a transplant from a latently infected donor.

In further embodiments, the invention relates to the treatment of cancer associated with elevated expression of HLA-G compared to non-cancerous cells, preferably with elevated expression of HLA-G and HLA-E compared to non-cancerous cells. The expression of HLA-G and HLA-E can be determined by well-known techniques, such as the nucleic acid and protein detection techniques based on qPCR and flow cytometry, which are known to the skilled person. Accordingly, it is possible to determine the expression level of HLA-G and/or HLA-E in a sample comprising pathological cells from a patient to a corresponding sample from a healthy individual or to reference values generated from corresponding samples. Such a comparison represents a routine analysis for a person skilled in the art. By means of such a comparison, it is possible to identify cancers that are susceptible to NKG2C+ NK cell cytotoxic activity.

Medical conditions associated with pathogenic cells expressing HLA-E and a peptide comprising an amino acid sequence according to SEQ ID NO 1-10 or 27-46, can be identified by a skilled person by standard laboratory methods. For example, HLA-E expression on the pathogenic cells can by analyzed by flow cytometry using HLA-E specific antibodies. The additional presence of peptides according to SEQ ID NO 1-10 or 27-46, can be determined for example by mass spectrometry or antibody mediated techniques. Furthermore, the presence of proteins that lead to the generation of these peptides, such as HLA-G or UL-40 of HCMV, can be determined by antibody based techniques such as ELISA or flow cytometry or even by RT-PCR detection expression of proteins encoding such leader peptides.

In the context of the present invention, the term “treatment of a tumor” relates to the treatment of all kinds of cancer, independent of whether the cancer is associated with the formation of a solid tumor or whether the cancer cells do not form a solid tumor, as it is the case for certain leukemias.

Cancer comprises a group of diseases that can affect any part of the body and is caused by abnormal cell growth and proliferation. These proliferating cells have the potential to invade the surrounding tissue and/or to spread to other parts of the body where they form metastasis. Worldwide, there were 14 million new cases of cancer and 8.2 million cancer related deaths in 2012 (World Cancer Report 2014). The majority of cancers is caused by environmental signals involving tobacco use, obesity and infections among others, while around 5-10% are genetic cases. Cancers can be classified into subcategories based on the cell of origin. The most common subcategories are carcinomas from epithelial cells, sarcomas from connective tissue and lymphomas and leukemias from hematopoietic cells. Cancer is associated with a high variety of local and systemic symptoms and cannot be cured in many cases. In light of the high number of new cancer patients and cancer related deaths novel treatment strategies are required.

Cancer according to the present invention refers to all types of cancer or neoplasm or malignant tumors found in mammals, including leukemias, sarcomas, melanomas and carcinomas. Either solid tumors and/or liquid tumors (such as leukemia or lymphoma) may be treated.

Leukemias include, but are not limited to acute nonlymphocytic leukemia, chronic lymphocytic leukemia, acute granulocytic leukemia, chronic granulocytic leukemia, acute promyelocytic leukemia, adult T-cell leukemia, aleukemic leukemia, a leukocythemic leukemia, 36oloney36ma leukemia, blast cell leukemia, bovine leukemia, chronic myelocytic leukemia, leukemia cutis, embryonal leukemia, eosinophilic leukemia, Gross' leukemia, hairy-cell leukemia, hemoblastic leukemia, hemocytoblastic leukemia, histiocytic leukemia, stem cell leukemia, acute monocytic leukemia, leukopenic leukemia, lymphatic leukemia, lymphoblastic leukemia, lymphocytic leukemia, lymphogenous leukemia, lymphoid leukemia, lymphosarcoma cell leukemia, mast cell leukemia, megakaryocytic leukemia, micromyeloblastic leukemia, monocytic leukemia, myeloblastic leukemia, myelocytic leukemia, myeloid granulocytic leukemia, myelomonocytic leukemia, Naegeli leukemia, plasma cell leukemia, plasmacytic leukemia, promyelocytic leukemia, Rieder cell leukemia, Schilling's leukemia, stem cell leukemia, subleukemic leukemia, and undifferentiated cell leukemia.

Sarcomas include, but are not limited to a chondrosarcoma, fibrosarcoma, lymphosarcoma, melanosarcoma, myxosarcoma, osteosarcoma, Abernethy's sarcoma, adipose sarcoma, liposarcoma, alveolar soft part sarcoma, ameloblastic sarcoma, botryoid sarcoma, chloroma sarcoma, chorio carcinoma, embryonal sarcoma, Wilms' tumor sarcoma, endometrial sarcoma, stromal sarcoma, Ewing's sarcoma, fascial sarcoma, fibroblastic sarcoma, giant cell sarcoma, granulocytic sarcoma, Hodgkin's sarcoma, idiopathic multiple pigmented hemorrhagic sarcoma, immunoblastic sarcoma of B cells, lymphoma, immunoblastic sarcoma of T-cells, Jensen's sarcoma, Kaposi's sarcoma, Kupffer cell sarcoma, angiosarcoma, leukosarcoma, malignant mesenchymoma sarcoma, parosteal sarcoma, reticulocytic sarcoma, Rous sarcoma, serocystic sarcoma, synovial sarcoma, and telangiectaltic sarcoma.

Melanomas include, but are not limited to include, for example, acral-lentiginous melanoma, amelanotic melanoma, benign juvenile melanoma, Cloudman's melanoma, S91 melanoma, Harding-Passey melanoma, juvenile melanoma, lentigo maligna melanoma, malignant melanoma, nodular melanoma, subungal melanoma, and superficial spreading melanoma.

Carcinomas include, but are not limited to acinar carcinoma, acinous carcinoma, adenocystic carcinoma, adenoid cystic carcinoma, carcinoma adenomatosum, carcinoma of adrenal cortex, alveolar carcinoma, alveolar cell carcinoma, basal cell carcinoma, carcinoma basocellulare, basaloid carcinoma, basosquamous cell carcinoma, bronchioalveolar carcinoma, bronchiolar carcinoma, bronchogenic carcinoma, cerebriform carcinoma, cholangiocellular carcinoma, chorionic carcinoma, colloid carcinoma, comedo carcinoma, corpus carcinoma, cribriform carcinoma, carcinoma en cuirasse, carcinoma cutaneum, cylindrical carcinoma, cylindrical cell carcinoma, duct carcinoma, carcinoma durum, embryonal carcinoma, encephaloid carcinoma, epiermoid carcinoma, carcinoma epitheliale, exophytic carcinoma, carcinoma exulcere, carcinoma fibrosum, gelatiniform carcinoma, gelatinous carcinoma, giant cell carcinoma, carcinoma gigantocellulare, glandular carcinoma, granulosa cell carcinoma, hair-matrix carcinoma, hematoid carcinoma, hepatocellular carcinoma, Hurthle cell carcinoma, hyaline carcinoma, hypernephroid carcinoma, infantile embryonal carcinoma, carcinoma in situ, intraepidermal carcinoma, intraepithelial carcinoma, Krompecher's carcinoma, Kulchitzky-cell carcinoma, large-cell carcinoma, lenticular carcinoma, carcinoma lenticulare, lipomatous carcinoma, lymphoepithelial carcinoma, carcinoma medullare, medullary carcinoma, melanotic carcinoma, mucinous carcinoma, carcinoma muciparum, carcinoma mucocellulare, mucoepidermoid carcinoma, carcinoma mucosum, mucous carcinoma, carcinoma myxomatodes, nasopharyngeal carcinoma, oat cell carcinoma, carcinoma ossificans, osteoid carcinoma, papillary carcinoma, periportal carcinoma, preinvasive carcinoma, prickle cell carcinoma, pultaceous carcinoma, renal cell carcinoma of kidney, reserve cell carcinoma, carcinoma sarcomatodes, scirrhous carcinoma, carcinoma scroti, signet-ring cell carcinoma, carcinoma simplex, small-cell carcinoma, colon carcinoma, spheroidal cell carcinoma, spindle cell carcinoma, carcinoma spongiosum, squamous carcinoma, squamous cell carcinoma, string carcinoma, carcinoma telangiectaticurn, carcinoma telangiectodes, transitional cell carcinoma, carcinoma tuberosum, tuberous carcinoma, verrucous carcinoma, and carcinoma villosum.

Additional cancers include, but are not limited to Hodgkin's Disease, Non-Hodgkin's Lymphoma, multiple myeloma, neuroblastoma, breast cancer, ovarian cancer, lung cancer, rhabdomyosarcoma, primary thrombocytosis, primary macroglobulinemia, small-cell lung tumors, primary brain tumors, stomach cancer, colon cancer, malignant pancreatic Moloney carcinoma, malignant carcinoid, urinary bladder cancer, premalignant skin lesions, testicular cancer, lymphomas, thyroid cancer, esophageal cancer, genitourinary tract cancer, malignant hypercalcemia, cervical cancer, endometrial cancer, adrenal cortical cancer, and prostate cancer.

In some embodiments, “tumor” shall include, without limitation, a prostate tumor, a pancreatic tumor, a squamous cell carcinoma, a breast tumor, a melanoma, a basal cell carcinoma, a hepatocellular carcinoma, a choloangiocellular carcinoma, testicular cancer, a neuroblastoma, a glioma or a malignant astrocytic tumor such as glioblastma multiforme, a colorectal tumor, an endometrial carcinoma, a lung carcinoma, an ovarian tumor, a cervical tumor, an osteosarcoma, a rhabdo/leiomyosarcoma, a synovial sarcoma, an angiosarcoma, an Ewing sarcoma/PNET and a malignant lymphoma. These include primary tumors as well as metastatic tumors (both vascularized and non-vascularized).

As used herein, the “patient” or “subject” may be a vertebrate animal. In the context of the present invention, the term “subject” includes both humans and animals, particularly mammals, and other organisms. In preferred embodiments the “patient” or “subject” may be a human.

In the context of the present invention, “treatment” or “therapy” generally means the achievement of a desired pharmacological effect and/or physiological effect. The effect may be prophylactic, to prevent all or part of a disease and/or symptom, e.g., by reducing the risk of a person having a disease or symptom, or it may be therapeutic, to partially or completely cure a disease and/or an undesirable effect of the disease.

In the context of the present invention, the term “therapy” includes all treatments of diseases or conditions in mammals, especially in humans, for example, the following treatments (i) to (iii): (i) preventing the onset of a disease, condition or symptom in a patient; (ii) inhibiting a symptom of a condition, i.e., preventing the progression of the symptom; (iii) ameliorating a symptom of a condition, i.e., inducing a regression of the disease or symptom.

In preferred embodiments of the invention, the nucleic acid molecule is administered in combination with an anti-cancer or an anti-viral therapy.

Anti-cancer therapies of the present invention comprise, without limitation, surgery, chemotherapy, radiotherapy, irradiation therapy, hormonal therapy, targeted therapy, immunotherapy, cell therapy and immune cell therapy.

In the context of the present invention, chemotherapy refers to a category of cancer treatment that uses one or more anti-cancer drugs (chemotherapeutic agents) as part of a chemotherapy regimen. Irradiation or radiation therapy or radiotherapy in the context of the present invention relates to a therapeutic approach using ionizing or ultraviolet-visible (UV/Vis) radiation, generally as part of cancer treatment to control or kill malignant cells such as cancer cells or tumor cells.

As used herein, “immunotherapy” comprises any kind of therapeutic approach or treatment directed against a tumor employing means of the immune system to negate or destroy tumor material. This includes, without limitation, immune checkpoint modulators, immune cell therapy, adoptive transfer of immune cells or other cells that modulate the immune response, modulation of the immune cells by small molecules or biopharmaceuticals such as monoclonal antibodies, cytokines, chemokines, and cancer treatment vaccines. Immunotherapies of the present invention further comprise administration of an antibody that binds specifically to a tumor-associated antigen, the administration of a cytokine or chemokine, the administration of a small molecule with anti-tumor immune-stimulating properties, the administration of tumor antigens and/or the administration of patient-derived tumor material.

### Vaccines, Adjuvants, Checkpoint Inhibitors:

The term “vaccine” in the context of the present invention relates to a biological preparation that provides active acquired immunity to a particular disease, such as cancer, a pathogen or an infectious agent, such as bacteria or viruses. In the context of the present invention, NKG2C+ NK cells may be considered to provide adapted or acquired immunity. A vaccine can contain an agent or antigen that resembles or is derived from a disease-causing microorganism. Vaccines can be made from weakened, attenuated, mutated or killed forms of the pathogen, its toxins or one of its surface proteins. The agent stimulates the body's immune system to recognize the agent as a threat, destroy it, and recognize and destroy any pathogens or structures comprising the agent or antigen of the vaccine that it later encounters. Vaccines can be prophylactic (example: to prevent or ameliorate the effects of a future infection by a natural or “wild” pathogen), or therapeutic, such as specific cancer vaccines.

In embodiment of the invention the nucleic acid molecule is administered in combination with an adjuvant. Preferably, the adjuvant enhances the production of pro-inflammatory cytokines. In embodiments, the nucleic acid molecule of the invention is administered in combination with pro-inflammatory cytokines.

As used herein, the term “adjuvant” relates to a compound or composition that is administered in combination with the nucleic acid molecule of the present invention, to enhance the effectiveness of the nucleic acid molecule. In general, an adjuvant is an agent that is given in addition to the primary or initial therapy to maximize its effectiveness. In the context of the present invention, the adjuvant is to be understood as an immunologic adjuvant. Adjuvants in immunology are often used to modify or augment the effects of a compound that modifies the immune system, such as the nucleic acid molecule of the present invention or a vaccine. In embodiments, the nucleic acid molecules of the invention may be regarded as a nucleic acid vaccine, e.g., RNA, mRNA or DNA vaccine. An immunological adjuvant stimulates the immune system to respond more vigorously to an immunological treatment. As a consequence, the combined treatment with an adjuvant provides increased immunity to a particular disease. It is believed that adjuvants accomplish this task by mimicking specific sets of evolutionarily conserved molecules, so called PAMPs, which include liposomes, lipopolysaccharide (LPS), molecular cages for antigen, components of bacterial cell walls, and endocytosed nucleic acids such as double-stranded RNA (dsRNA), single-stranded DNA (ssDNA), and unmethylated CpG dinucleotide-containing DNA. Because immune systems have evolved to recognize these specific antigenic moieties, the presence of an adjuvant can greatly increase the innate immune response to the antigen by augmenting the activities of dendritic cells (DCs), lymphocytes, and macrophages by mimicking a natural infection. Furthermore, the use of such adjuvants that are mimicking PAMPs leads to the production of pro-inflammatory cytokines.

Vaccines are administered in a manner compatible with the dosage formulation and in an amount that is therapeutically effective, protective and immunogenic. The amount to be administered depends on the individual being treated, e.g., the ability of the individual's immune system to synthesize antibodies and, if appropriate, to mount a cell-mediated immune response. The exact amount of vaccine or immunogenic composition to be administered is at the discretion of the responsible physician. However, suitable dosage ranges are readily determined by one skilled in the art and may be on the order of micrograms of the nucleic acid molecule of the invention. In embodiments suitable dosage ranges may be between 30-50 μg applied in 500 μl (60-100 μg/mL), dosage ranges between 10-200 μg/mL, 25-100 g/mL, 25-50 μg/mL, 50-150 g/mL, or 10, 15, 20, 25, 30, 35, 40, 45, 50, 55, 65, 70, 75, 80, 85, 90, 95, 100, 105, 110, 115, 120, 125, 130, 135, 140, 145, 150 μg/mL or dosages of 5, 10, 15, 20, 25, 30, 35, 40, 45, 50, 55, 65, 70, 75, 80, 85, 90, 95, or 100 μg.

Suitable schedules for initial administration and booster doses are also variable, but may include an initial administration followed by subsequent administrations (“booster” administration). The dosage of the vaccine may also depend on the route of administration and will vary according to the size of the host.

Immunological adjuvants for use in the context of the present invention comprise, without limitation, inorganic adjuvants, such as aluminium salts (aluminium phosphate and aluminium hydroxide), squalene, AS02, AS03, AS04, oil-based adjuvants (emulsions), MF59, QS21, cytokines, virosomes, pathogen components, such as monophosphryl lipid A, Poly(IC:C) and CpG DNA adjuvants.

As known in the art, a pro-inflammatory cytokine or an immune response-stimulating cytokine is to be understood as a cytokine that leads to or produces either directly or indirectly the induction, activation and/or enhancement of an immune response, preferably directed against an antigen, for example a tumor or CMV antigen.

Cytokines are a diverse group of non-antibody proteins that act as mediators between cells. Cytokines are currently being clinically used as biological response modifiers for the treatment of various disorders. The term cytokine is a general term used to describe a large group of proteins. Particular kinds of cytokines may include Monokines, namely cytokines produced by mononuclear phagocytic cells, Lymphokines, namely cytokines produced by activated lymphocytes, especially Th cells, Interleukins, namely cytokines that act as mediators between leukocytes and

Chemokines, namely small cytokines primarily responsible for leucocyte migration. Cytokine signaling is flexible and can induce both protective and damaging responses. They can produce cascades, or enhance or suppress production of other cytokines. Despite the various roles of cytokines, a skilled person is aware of which cytokines may be considered as immune response stimulating and therefore applied in the treatment of a tumor disease as described herein.

Cytokines have the ability to modulate immune responses and are often utilized by a tumor to allow it to grow and manipulate the immune response. These immune-modulating effects allow them to be used as drugs to provoke an immune response against the tumor. Chemokines refer to a sub-group of cytokines (signaling proteins) secreted by cells. Chemokines have the ability to induce directed chemotaxis in nearby responsive cells; they are chemotactic cytokines.

Immune-response stimulatory or immune response-modulatory cytokines and chemokines comprise, without limitation, type 1 interferons (IFN alpha and IFN beta), type 2 (IFN gamma), type III interferons (IFN lambda), IFN gamma, TNF-alpha, IL-1, IL-2, IL-12, IL-18, IL-23, IL-15 and IL-21, CXCL-8, CCL2, CCL3, CCL4, CCL5, CCL11 and CXCL10, CXCL1, CXCL2, CCL2, CCL1, CCL22, CCL17, CXCL13, CX3CL1, SDF-1, CXCL12, CCL23, MIP-3, MPIF-1, CCL19, MIP-3-beta and MIP-1β.

The terms “stimulation” and “activation” of the “immune system” or of an “immune response” may be used interchangeably.

In preferred embodiments of the invention, the nucleic acid molecule is administered in combination with a check point inhibitor, preferably an inhibitor of a receptor selected from the group comprising LILRB1, inhibitory KIRs, NKG2A, PD-1, CTLA-4, TIM-3, TIGIT and LAG-3.

Immune checkpoint molecules are molecules in the immune system that either turn up a signal (co-stimulatory molecules) or turn down a signal provided to immune effector cells. Thus, immune checkpoint molecules can be subdivided into co-stimulatory checkpoint molecules or co-inhibitory checkpoint molecules. Co-stimulatory checkpoint molecules include co-stimulatory lymphocyte receptors, which are lymphocyte surface-receptors that can lead to an activation or stimulation of lymphocyte effector functions. Co-inhibitory checkpoint molecules include co-inhibitory lymphocyte receptors, which are lymphocyte surface-receptors that can lead to an inhibition of lymphocyte effector functions.

An inhibitor of a receptor prevents the generation of a signal by the respective receptor. Accordingly, an inhibitor of a co-inhibitory lymphocyte receptor is a molecule that prevents the activation of the respective receptor and thereby prevents the generation of an inhibitory signal. Conversely, an activator of a receptor induces the generation of a signal by the respective receptor and an activator of a co-stimulatory lymphocyte receptor leads to the generation of a stimulatory signal. Checkpoint modulators are molecules that interfere with the activity of immune checkpoint molecules, either by stimulating or inhibiting the activity of immune checkpoint molecules.

Lymphocyte-stimulating checkpoint modulators are molecules that lead to an activation of lymphocytes, preferably effector T cells, either through activation of a co-stimulatory checkpoint molecule, or through inhibition of co-inhibitory checkpoint molecules. Checkpoint modulators can be naturally occurring molecules or engineered molecules with the respective function interfering with or modulating the activity of an immune checkpoint molecule. Checkpoint modulators include, for example, antibodies or antibody-fragments activity directed against immune checkpoint molecule with agonistic or antagonistic, and ligands or modified ligands of immune checkpoint molecules.

Co-inhibitory checkpoint molecules comprise, without limitation, LILRB1, A2AR, B7-H3, B7-H4, BTLA, CTLA-4, IDO, KIR, LAG3, PD-1, TIM-3, TIGIT and VISTA.

Leukocyte immunoglobulin-like receptor subfamily B member 1 (LILRB1) is a protein that in humans is encoded by the LILRB1 gene. The protein belongs to the subfamily B class of LIR receptors which contain two or four extracellular immunoglobulin domains, a transmembrane domain, and two to four cytoplasmic immunoreceptor tyrosine-based inhibitory motifs (ITIMs). The receptor is expressed on immune cells where it binds to MHC class I molecules on antigen-presenting cells and transduces a negative signal that inhibits stimulation of an immune response. It is involved in the control of inflammatory responses and cytotoxicity to help focus the immune response and limit autoreactivity.

A2AR (Adenosine A2A receptor) is regarded as an important checkpoint in cancer therapy because adenosine in the immune microenvironment, leading to the activation of the A2a receptor, is negative immune feedback loop and the tumor microenvironment has relatively high concentrations of adenosine.

B7-H3, also called CD276, was originally understood to be a co-stimulatory molecule but is now regarded as co-inhibitory. MacroGenics is working on MGA271 (Enoblituzumab), which is an Fc-optimized monoclonal antibody that targets B7-H3.

B7-H4 (or VTCN1) is expressed by tumor cells and tumor-associated macrophages and plays a role in tumor evasion.

BTLA (B and T Lymphocyte Attenuator, also called CD272) is a co-inhibitory receptor, which has HVEM (Herpesvirus Entry Mediator) as its ligand. Surface expression of BTLA is gradually downregulated during differentiation of human CD8+ T cells from the naïve to effector cell phenotype, however tumor-specific human CD8+ T cells express high levels of BTLA.

CTLA-4 (Cytotoxic T-Lymphocyte-Associated protein 4, also called CD152) is expressed on Treg cells and serves to control T cell proliferation. CTLA-4 (CD152) is a protein receptor functioning as an immune checkpoint and is expressed by activated T cells and transmits an inhibitory signal to T cells. CTLA4 is homologous to the T-cell co-stimulatory protein CD28, and both molecules bind to CD80 and CD86 (B7-1 and B7-2 respectively), on antigen-presenting cells. CTLA-4 has a greater affinity and avidity to CD80 and CD86 with than CD28. CTLA4 transmits an inhibitory signal to T cells. Antagonistic antibodies directed against CTLA4 include ipilimumab and tremelimumab.

IDO (Indoleamine 2,3-dioxygenase) is a tryptophan catabolic enzyme with immune-inhibitory properties. Another important molecule is TDO, tryptophan 2,3-dioxygenase. IDO is known to suppress T and NK cells, generate and activate Tregs and myeloid-derived suppressor cells, and promote tumor angiogenesis.

KIR (Killer-cell Immunoglobulin-like Receptor) is a receptor for MHC Class I molecules on Natural Killer cells. Lirilumab is a monoclonal antibody to KIR.

LAG-3 (Lymphocyte Activation Gene-3) works to suppress an immune response by action to Tregs as well as direct effects on CD8+ T cells.

PD-1 (Programmed Death 1, or CD279) is a cell surface receptor that plays an important role in down-regulating the immune system and promoting self-tolerance by suppressing T cell inflammatory activity. PD-1 has two ligands, PD-L1 and PD-L2. An advantage of targeting PD-1 is that it can restore immune function in the tumor microenvironment. PD-L1, the ligand for PD1, is highly expressed in several cancers and can lead to the inhibition of anti-cancer immune response by T cells. A number of cancer immunotherapy agents that target the PD-1 receptor have been developed, including the antagonistic antibodies nivolumab, (Opdivo-Bristol Myers Squibb), Pembrolizumab (Keytruda, MK-3475, Merck), Pidilizumab (CT-011, Cure Tech) and BMS-936559 (Bristol Myers Squibb). Both Atezolizumab (MPDL3280A, Roche) and Avelumab (Merck KgaA, Darmstadt, Germany & Pfizer) are monoclonal antibodies directed against PD-L1, the ligand of PD-1.

TIM-3 (T-cell Immunoglobulin domain and Mucin domain 3) expresses on activated human CD4+ T cells and regulates Th1 and Th17 cytokines. TIM-3 acts as a negative regulator of Th1/Th17 function by triggering cell death upon interaction with its ligand, galectin-9.

VISTA (V-domain Ig suppressor of T cell activation) is a protein that is primarily expressed on hematopoietic cells so that consistent expression of VISTA on leukocytes within tumors may allow VISTA blockade to be effective across a broad range of solid tumors.

TIGIT (T cell immunoreceptor with Ig and ITIM domains, also called WUCAM and Vstm3) is an immune receptor present on some T cells and Natural Killer Cells and regulates T cell mediated immunity. TIGIT could bind to CD155 on DCs and macrophages with high affinity and to CD112 with lower affinity.

Co-stimulatory checkpoint molecules comprise, without limitation, HVEM, CD27, CD40, OX40, GITR, CD137, CD28 and ICOS.

### Gene Therapy, Nucleic Acids, Polypeptides:

In embodiments of the invention, the nucleic acid molecule for use as a medicament is administered by or comprised within a vector comprising or encoding the nucleic acid molecule of the present invention. Therefore, the present invention encompasses gene therapy comprising the administration of a therapeutic gene encoding the polypeptide described herein.

The term gene therapy preferably refers to the transfer of nucleic acids into a subject to treat a disease. The person skilled in the art knows strategies to perform gene therapy using gene therapy vectors. Such gene therapy vectors are optimized to deliver foreign DNA into the host cells of the subject. In a preferred embodiment the gene therapy vectors may be a viral vector.

Viruses have naturally developed strategies to incorporate DNA into the genome of host cells and may therefore be advantageously used. Preferred viral gene therapy vectors may include but are not limited to retroviral vectors such as Moloney murine leukemia virus (MMLV), adenoviral vectors, lentiviral, adenovirus-associated viral (AAV) vectors, pox virus vectors, vaccinia virus, herpes simplex virus vectors or human immunodeficiency virus vectors (HIV-1). Furthermore, the vector of the present invention may be an attenuated HCMV virus or vector, which has been genetically modified to be less harmful to the infected host than the unmodified wild-type version of the virus. The viral vectors of the invention are preferably genetically modified.

However also non-viral vectors may be preferably used for the gene therapy such as plasmid expression vectors driven by eukaryotic promoters or liposomes encapsulating the transfer DNA. Furthermore, preferred gene therapy vectors may also refer to methods to transfer of the DNA such as electroporation or direct injection of nucleic acids into the subject. Moreover, it may be preferred that the gene therapy vectors for example a viral gene therapy vector is adapted to target suitable cells of the body, such as for example bone marrow cells, hematopoietic cells, or immune cells or progenitor cells of immune cells, preferably NK cells, NK cell progenitors or NK cell subsets, such as NKG2C+ NK cells. To this end the viral capsid may be conjugated with ligands binding to the specific target cells, such as bone marrow cells, such as hematopoietic cells, or immune cells or progenitor cells of immune cells, preferably NK cells, NK cell progenitors or NK cell subsets, such as NKG2C+ NK cells, such as monoclonal antibodies. It may also be preferred that the viral gene therapy vectors are genetically modified using inducible promoters or promoters that are specific for the target cells of interest, such as bone marrow cells, such as hematopoietic cells, or immune cells or progenitor cells of immune cells, preferably NK cells, NK cell progenitors or NK cell subsets, such as NKG2C+ NK cells, to enhance the expression of the nucleic acid specifically in the target cells. Preferred gene therapy vectors may therefore comprise vectors for an inducible or conditional expression of the polypeptides. The person skilled in the art knows how to choose preferred gene therapy vectors according to the need of application as well as the methods on how to implement the nucleic acid into the gene therapy vector. (P. Seth et al., 2005, N. Koostra et, al. 2009., W. Walther et al. 2000, Waehler et al. 2007).

As used herein, “nucleic acid” shall mean any nucleic acid molecule, including, without limitation, DNA, RNA and hybrids or modified variants thereof. An “exogenous nucleic acid” or “exogenous genetic element” or “heterologous nucleic acid” relates to any nucleic acid introduced into the cell, which is not a component of the cells “original” or “natural” genome. Exogenous nucleic acids may be integrated or non-integrated, or relate to stably transfected nucleic acids. The terms exogenous and heterologous may in embodiments be used interchangeably.

Hence the term “nucleic acid molecule” might likewise refer to a deoxyribonucleic acid (DNA) molecule and a ribonucleic acid (RNA) molecule. In embodiments the term “nucleic acid molecule” relates to an RNA molecule. In embodiments the term “nucleic acid molecule” relates to a messenger RNA (mRNA) molecule. The nucleic acid molecule can be single-stranded or double-stranded, or partially single-stranded and partially double-stranded. In some preferred embodiments the nucleic acid molecule is single-stranded RNA. The nucleic acid molecule may in embodiments comprise or consist of an artificial, a partially artificial, and/or an engineered nucleic acid sequence. Herein, preferably a nucleic acid molecule comprises a nucleic acid sequence.

Herein any nucleic acid sequence given as DNA or cDNA sequence, comprising the nucleobases A, T, C and/or G, may also comprise or refer to a corresponding RNA sequence, comprising the nucleobases A, U, C and/or G, and may in embodiments represent the cDNA sequence of said RNA sequence. The same applies for each RNA sequence, which may in embodiments also comprise or refer to the corresponding (c) DNA sequence. In other words, any DNA sequence disclosed herein also comprises the corresponding RNA sequence (having T exchanged for U), and vice versa. Nucleic acid sequences disclosed herein are also intended to comprise the nucleic acid sequence complementary thereto.

Ribonucleic acids (RNAs) are linear molecules composed of four different kinds of ribonucleotide bases, namely Cytosine (C), Adenine (A), Guanine (G) and Uracil (U). A ribonucleotide base is composed of three building blocks, namely a ribose sugar, a phosphate group, and a nitrogenous base. In an RNA molecule the ribonucleotide bases are linked through phosphodiester bonds. RNA is generally single-stranded, contrary to DNA, which commonly occurs as single or double-stranded molecule. During the process of transcription RNA is synthesized from DNA by an RNA polymerase enzyme, wherein the nucleic acid sequence of the obtained RNA molecule is complementary to the DNA template. During the process of translation RNA molecules are translated into proteins. Different types of RNA are known, such as messenger RNA (mRNA), transfer RNA (IRNA), and ribosomal RNA (rRNA). In some preferred embodiments herein the nucleic acid molecule according to the invention is an mRNA molecule.

Deoxyribonucleic acids (DNAs) are linear molecules composed of four different kinds of ribonucleotide bases, namely Cytosine (C), Adenine (A), Guanine (G) and thymine (T). DNA is in nature rarely present in form of a single strand, but of two strands that form a double helix. The DNA double helix is stabilized mainly by two forces: Hydrogen bonds between the nucleotides and base-stack interactions between the aromatic nucleobases. Generally, a DNA sequence is transcribed by a Polymerase enzyme into an RNA sequence.

Herein a “vector” refers to a nucleic acid molecule capable of transporting another nucleic acid to which it has been linked. The vector can integrate into a host DNA or be capable of autonomous replication. Non-limiting examples of a vector are a plasmid, cosmid, or viral vector. In embodiments a vector can comprise a nucleic acid in a form suitable for expression of the nucleic acid in a host cell.

The present invention may in embodiments relate to a nucleic acid molecule encoding a peptide of the invention. The nucleic acid according to the invention and preferred embodiments thereof, in particular a nucleic acid molecule encoding a polypeptide of the present invention, is particularly efficient for gene therapy due to a high therapeutic potential at a small size. This ensures a stable integration at high expression levels over extended periods of times.

In one embodiment the invention relates to a cell for use as a medicament to expand and/or activate NKG2C+ natural killer (NK) cells in the treatment and/or prevention of a medical condition treatable by the effector function of said NKG2C+ NK cells. Therein the cell may be a NKG2C+ NK cell generated by the method of the present invention for cultivating and/or expanding NKG2C+ natural killer (NK) cells, or a cell, which is genetically modified and comprises an exogenous nucleic acid region encoding for a polypeptide according to the invention or preferred embodiments thereof and wherein the exogenous nucleic acid region is operably linked to a promoter.

The person skilled in the art knows how to genetically modify cells in order to express the polypeptides according to the invention. In certain embodiments the modification may be the delivery of the nucleic acid molecule according to the invention to a cell and/or tissue, such that the polypeptides according to the invention may be expressed in said cells and/or tissue. Advantageously by expressing the therapeutically effective polypeptides according to the invention the cells may act as bio pump or drug factory that continuously expresses and provides the polypeptides to the subject. Thereby the amount of the polypeptides can be held at a therapeutic level over long periods. The person skilled in the art knows which cells may be preferably used to this end. In a preferred embodiment the cells are stem cells, characterized by a stable expression of the polypeptides. Stem cells may include but are not limited to, embryonic stem cells such as early embryonic stem cells and blastocyst embryonic stem cells; fetal stem cells; umbilical cord stem cells; and adult stem cells such as mesenchymal stem cells, hematopoietic stem cells, endothelial stem cells, peripheral blood stem cells, and multipotent somatic stem cells.

The cells may migrate to the site of NK cells, NK progenitor cells or NKG2C+ NK cells in order to locally express the polypeptides of the invention in vicinity of the cells to be activated and/or expanded. Advantageously the cells may however in embodiments also be transplanted at a different location as the polypeptides of the invention can also be transported by the vascular system throughout the body of the subject. In embodiments local administration of the cells e.g., by a subcutaneous injection may therefore contribute in a systemic manner largely irrespective of the location of the cells within the body of the subject.

### Administration, Compositions:

In one embodiment the nucleic acid molecules for use as a medicament as described herein are characterized by introducing a therapeutically effective number of said nucleic acid molecules either directly or comprised by a suitable vector as described herein, such as a viral vector or a cell carrying a nucleic acid molecule of the invention, e.g., encoding the peptide of the invention, to a subject within a biocompatible matrix. Preferred materials for the biocompatible matrix are agarose, carrageenan, alginate, chitosan, gellan gum, hyaluronic acid, collagen, cellulose and its derivatives, gelatin, elastin, epoxy resin, photo cross-linkable resins, polyacrylamide, polyester, polystyrene and polyurethane or polyethylene glycol (PEG). It is further preferred that the biocompatible matrix is a semi-permeable hydrogel matrix and the peptides, nucleic acid molecules or vectors carrying the peptide and/or a nucleic acid molecule encoding the peptide are entrapped by said matrix. Advantageously the biocompatible matrix allows for an efficient diffusion of nutrients, oxygens and other biomolecules to ensure a long lasting persistence of the nucleic acid molecules, peptides or vectors carrying the peptide and/or a nucleic acid molecule encoding the peptide, while immobilizing the nucleic acid molecules, peptides or vectors carrying the peptide and/or a nucleic acid molecule encoding the peptide. Thereby the cells can be concentrated at preferred locations within the subject. For instance, the nucleic acid molecules, peptides or vectors carrying the peptide and/or a nucleic acid molecule encoding the peptide cells can be transplanted subcutaneously and/or in proximity of diseased regions of the subject.

In a preferred embodiment the invention further relate to pharmaceutical composition for use as a medicament, preferably to expand and/or activate NKG2C+ natural killer (NK) cells in the treatment and/or prevention of a medical condition associated with pathogenic cells expressing HLA-E and a peptide comprising an amino acid sequence according to SEQ ID NO 1-10 or 27-46 as described herein, wherein the pharmaceutical composition comprises the nucleic acid molecule or polypeptide, the nucleic acid, the gene therapy vector and/or the cell according to the invention, and optionally a pharmaceutically accepted carrier. Preferably the pharmaceutical composition is administered to the subject at a therapeutically effective amount at any administration route as described herein. In the context of the present invention, a cell comprising or encoding a nucleic acid molecule and/or peptide of the present invention may be considered a vector.

In a preferred embodiment the pharmaceutical composition for use as a medicament as described herein is administered by introducing a therapeutically effective amount of the composition into the blood stream of a subject. In a further preferred embodiment the pharmaceutical composition for use as a medicament as described herein is administered locally, for example by administration to a site of the subject's body in proximity to a site where pathogenic cells expressing HLA-E and a peptide comprising an amino acid sequence according to SEQ ID NO 1-10 or 27-46 are localized. As used herein, in “proximity with” a tissue/site includes, for example, within 50 mm, 20 mm, 10 mm, 5 mm, within 1 mm of the tissue, within 0.5 mm of the tissue and within 0.25 mm of the tissue/site.

The polypeptides, nucleic acid molecules, gene therapy vectors or cells described herein may comprise different types of carriers depending on whether they are to be administered in solid, liquid or aerosol form, and whether they need to be sterile for such routes of administration as injection.

The active agent of present invention can be administered intravenously, intradermally, intraarterially, intraperitoneally, intralesionally, intracranially, intraarticularly, intraprostaticaly, intrapleurally, intratracheally, intranasally, intravitreally, intravaginally, intrarectally, topically, intratumorally, intramuscularly, intraperitoneally, subcutaneously, subconjunctival, intravesicularlly, mucosally, intrapericardially, intraumbilically, intraocularally, orally, topically, locally, inhalation (e.g., aerosol inhalation), injection, infusion, continuous infusion, directly, via a catheter, via a lavage, in cremes, in lipid compositions (e.g., liposomes), locally applied by sponges or by other method or any combination of the forgoing as would be known to one of ordinary skill in the art (see, for example, Remington's Pharmaceutical Sciences, 18th Ed. Mack Printing Company, 1990, incorporated herein by reference).

Such administering can also be performed, for example, once, a plurality of times, and/or over one or more extended periods. A single injection is preferred, but repeated injections over time (e.g., quarterly, half-yearly or yearly) may be necessary in some instances. Such administering is also preferably performed using an admixture of polypeptides, nucleic acids, gene therapy vectors or cells and a pharmaceutically acceptable carrier. Pharmaceutically acceptable carriers are well known to those skilled in the art and include, but are not limited to, 0.01-0.1 M and preferably 0.05 M phosphate buffer or 0.8% saline.

As used herein, “pharmaceutically acceptable carrier” includes any and all solvents, dispersion media, coatings, antibacterial and antifungal agents, isotonic and absorption delaying agents and the like. The use of such media and agents for pharmaceutical active substances is well known in the art. Except insofar as any conventional media or agent is incompatible with the active ingredient, its use in the therapeutic compositions is contemplated. Supplementary active ingredients can also be incorporated into the compositions.

Additionally, such pharmaceutically acceptable carriers can be aqueous or non-aqueous solutions, suspensions, and emulsions, most preferably aqueous solutions. Aqueous carriers include water, alcoholic/aqueous solutions, emulsions and suspensions, including saline and buffered media. Parenteral vehicles include sodium chloride solution, Ringer's dextrose, dextrose and sodium chloride, lactated Ringer's and fixed oils. Intravenous vehicles include fluid and nutrient replenishers, electrolyte replenishers such as Ringer's dextrose, those based on Ringer's dextrose, and the like. Fluids used commonly for i.v. administration are found, for example, in Remington: The Science and Practice of Pharmacy, 20^{th} Ed., p. 808, Lippincott Williams S-Wilkins (2000). Preservatives and other additives may also be present, such as, for example, antimicrobials, antioxidants, chelating agents, inert gases, and the like.

The phrase “pharmaceutically acceptable” refers to molecular entities and compositions that do not produce an allergic or similar untoward reaction when administered to a human. The preparation of an aqueous composition that contains a protein as an active ingredient is well understood in the art. Typically, such compositions are prepared as injectables, either as liquid solutions or suspensions; solid forms suitable for solution in, or suspension in, liquid prior to injection can also be prepared. The preparation can also be emulsified.

The composition can be formulated in a neutral or salt form. Pharmaceutically acceptable salts, include the acid addition salts (formed with the free amino groups of the protein) and which are formed with inorganic acids such as, for example, hydrochloric or phosphoric acids, or such organic acids as acetic, oxalic, tartaric, mandelic, and the like. Salts formed with the free carboxyl groups can also be derived from inorganic bases such as, for example, sodium, potassium, ammonium, calcium, or ferric hydroxides, and such organic bases as isopropylamine, trimethylamine, histidine, procaine and the like. Upon formulation, solutions will be administered in a manner compatible with the dosage formulation and in such amount as is therapeutically effective. The formulations are easily administered in a variety of dosage forms such as injectable solutions, drug release capsules and the like. As used herein, a “therapeutically effective amount” for the pharmaceutical composition includes, without limitation, the following amounts and ranges of amounts:

Excipients, as an example of pharmaceutically acceptable carrier, for liquid formulations intended for injection are known in the art and can be selected appropriately by a skilled person. Excipients have been used to increase the stability of a wide range of protein and peptide-based formulations by reducing protein dynamics and motion, increasing the conformational stability of vaccine gene or vaccine protein especially at high concentrations and inhibiting interface-dependent aggregation. Excipients usually inhibit aggregation and protects the protein by adsorbing to the air-liquid interface; for example, the use of surfactants (e.g., polysorbate 20 and 80), carbohydrates (e.g., cyclodextrin derivatives) and amino acids (e.g., arginine and histidine) can help prevent aggregation by this mechanism. Cyclodextrin has been reported to stabilize commercially available antibody-based drugs in a hydrogel formulation. Some of the generally recognized as safe (GRAS) excipients include pluronic F68, trehalose, glycine and amino acids such as arginine, glycine, glutamate and histidine, which are found in a number of commercial protein therapeutic products. By way of example, bevacizumab, 25 mg/mL, contains trehalose dehydrate, sodium phosphate and polysorbate 20. Excipients in subcutaneous trastuzumab, 600 mg, are rHuPH20, histidine hydrochloride, histidine, trehalose dehydrate, polysorbate 20, methionine and water for injection.

When a therapeutically effective amount of the active substance (nucleic acid molecule) of the invention is administered by intramuscular, intravenous, cutaneous or subcutaneous injection, the active substance may be in the form of a solution, preferably a pyrogen-free, parenterally acceptable aqueous solution. The preparation of such parenterally acceptable solutions, having due regard to pH, isotonicity, stability, and the like, is within the skill in the art. A preferred pharmaceutical composition for intramuscular, intravenous, cutaneous, or subcutaneous injection should contain, in addition to the active substance, an isotonic vehicle such as Sodium Chloride Injection, Ringer's Injection, Dextrose Injection, Dextrose and Sodium Chloride Injection, Lactated Ringer's Injection, or other vehicle as known in the art. The pharmaceutical composition of the present invention may also contain stabilizers, preservatives, buffers, antioxidants, or other additives known to those of skill in the art.

For a composition comprising a nucleic acid molecule or polypeptide according to the invention or preferred embodiment thereof: (i) from about 1×10⁻³ to about 1×10⁶ μg/kg body weight; (ii) from about 1×10⁻² to about 1×10⁵ μg/kg body weight; (iii) from about 1×10⁻¹ to about 1×10⁴ μg/kg body weight; (iv) from about 1×10⁻¹ to about 1×10³ μg/kg body weight; (v) from about 1×10⁻¹ to about 1×10² μg/kg body weight; (vi) from about 1×10⁻¹ to about 0.5×10² μg/kg body weight; (vii) about 1×10⁻² μg/kg body weight; (viii) about 1×10¹ μg/kg body weight; (ix) about 10 μg/kg body weight (x) about 1×10² μg/kg body weight; (xi) about 5×10³ μg/kg body weight.

For a composition comprising cells according to the invention or preferred embodiment thereof: (i) from about 1×10² to about 1×10⁸ cells/kg body weight; (ii) from about 1×10³ to about 1×10⁷ cells/kg body weight; (iii) from about 1×10⁴ to about 1×10⁶ cells/kg body weight; (iv) from about 1×10⁴ to about 1×10⁵ cells/kg body weight; (v) from about 1×10⁵ to about 1×10⁶ cells/kg body weight; (vi) from about 5×10⁴ to about 0.5×10⁵ cells/kg body weight; (vii) about 1×10³ cells/kg body weight; (viii) about 1×10⁴ cells/kg body weight; (ix) about 5×10⁴ cells/kg body weight; (x) about 1×10⁵ cells/kg body weight; (xi) about 5×10⁵ cells/kg body weight; (xii) about 1×10⁶ cells/kg body weight; and (xiii) about 1×10⁷ cells/kg body weight.

Human body weights envisioned include, without limitation, about 5 kg, 10 kg, 15 kg, 30 kg, 50 kg, about 60 kg; about 70 kg; about 80 kg, about 90 kg; about 100 kg, about 120 kg and about 150 kg.

Dosages of the viral gene therapy vector will depend primarily on factors such as the condition being treated, the selected gene, the age, weight and health of the patient, and may thus vary among patients. For example, a therapeutically effective human dosage of the viral vectors may be preferably in the range of from about 1 to about 1000 ml, preferably 10 to 100 ml, preferably 20 to 50 ml of saline solution containing concentrations of from about 1×10⁵ to 1×10¹² preferably 1×10⁶ to 1×10¹¹ more preferably 1×10⁷ to 1×10¹⁰ plaque forming units (pfu)/ml viruses. The dosage will be adjusted to balance the therapeutic benefit against any side effects. The levels of expression of the selected gene can be monitored to determine the selection, adjustment or frequency of dosage administration.

As used herein “inducible expression” or “conditional expression” relates to a state, multiple states or system of an expression of the polypeptide according to the invention, wherein the polypeptide is preferably not expressed, or in some embodiments expressed at negligible or relatively low levels, unless there is the presence of one or more molecules (an inducer) or other set of conditions in the cell that allows for polypeptide expression. Inducible promoters may relate to either naturally occurring promoters that are expressed at a relatively higher level under particular biological conditions, or to other synthetic promoters comprising any given inducible element. Inducible promoters may refer to those induced by particular tissue- or micro-environments or combinations of biological signals present in particular tissue- or micro-environments, or to promoters induced by external factors, for example by administration of a small drug molecule or other externally applied signal.

“Combined administration” may relate to concurrent and/or sequential administration of the nucleic acid molecule or polypeptide of the invention prior to, during and/or subsequent to said adjuvant, check point inhibitor and/or further treatment. Combined treatment shall also include a combination treatment regimens comprising multiple administrations of either therapeutic component of the treatment. Further embodiments of combined administration are provided herein.

Combined administration encompasses simultaneous treatment, co-treatment or joint treatment, and includes the administration of separate formulations of the nucleic acid molecule or polypeptide of the present invention with said adjuvant, check point inhibitor and/or further treatment, whereby treatment may occur within minutes of each other, in the same hour, on the same day, in the same week or in the same month or within 3 months as one another. Sequential administration of any given combination of combined agents is also encompassed by the term “combined administration”. A combination medicament, comprising one or more of said nucleic acid molecule or polypeptide of the invention, said adjuvant, check point inhibitor and/or further treatment, may also be used in order to co-administer the various components in a single administration or dosage.

As used herein, the term “sample” is a biological sample that is obtained or isolated from the patient or subject. “Sample” as used herein may, e.g., refer to a sample of bodily fluid or tissue obtained for the purpose of diagnosis, prognosis, or evaluation of a subject of interest, such as a patient. Preferably herein, the sample is a sample of a bodily fluid, such as blood, serum, plasma, cerebrospinal fluid, urine, saliva, sputum, pleural effusions, cells, a cellular extract, a tissue sample, a tissue biopsy, a stool sample and the like. In the context of the present invention, any kind of sample comprising pathogenic cells potentially expressing HLA-E and a peptide comprising an amino acid sequence according to SEQ ID NO 1-10 or 27-46, such as cancer cells or cells that may comprise reactivated HCMV.

As used herein, the terms “comprising” and “including” or grammatical variants thereof shall be understood to specify the specified features, integers, steps, or components, without excluding the addition of one or more additional features, steps, integers, components, or groups thereof. This term includes the terms “consisting of” and “consisting essentially of.” Thus, the terms “consisting of”/“including”/“with” mean that any additional component (or features, steps, integers, and the like) may be used or included.

### FIGURES

The invention is further described by the following figures. These are not intended to limit the scope of the invention but represent preferred embodiments of aspects of the invention provided for greater illustration of the invention described herein.

### BRIEF DESCRIPTION OF THE FIGURES

FIG. 1: mRNA-UL40 is not sufficient for peptide presentation on HLA-E

FIG. 2: mRNA-LFL prototype induces potent activation of NKG2C+ NK cells.

FIG. 3: Peptide stabilization of HLA-E surface expression.

FIG. 4: NK cell stimulation assays.

FIG. 5: mRNA-LFL prototype induces activation of NKG2C⁺ NK cells in an autologous setting.

### DETAILED DESCRIPTION OF THE FIGURES

FIG. 1: mRNA-UL40 is not sufficient for peptide presentation on HLA-E. (A) Initial mRNA design containing the complete HCMV UL40 (“mRNA-UL40”) with the VMAPRTLFL peptide variant. (B) HLA-E surface upregulation (left) and eGFP expression (right) 16 h after electroporation of 400,000 K562/HLA-E cells with 500 ng of mRNA-UL40 or a parallel construct encoding for eGFP (“mRNA-eGFP”). Synthetic VMAPRTLFL peptide was pulsed at 300 μM as positive control for HLA-E upregulation.

FIG. 2: mRNA-LFL prototype induces potent activation of NKG2C⁺ NK cells. (A) mRNA design. (B) HLA-E surface upregulation 6 h and 24 h after electroporation of 400,000 K562/HLA-E cells with 10 ng, 100 ng or 500 ng of mRNA-LFL. (C) Activation of NKG2C⁺ NK cells after 6 h of co-culture with K562/HLA-E target cells electroporated with mRNA-LFL compared to a control mRNA encoding eGFP (500 ng/400,000 cells). (D) Comparison of activation by target cells pulsed with 300 μM of synthetic VMAPRTLFL peptide (“Peptide”) or electroporated with mRNA-LFL. Activation is displayed as the mean difference between these conditions and control conditions (inactive VMAPQSLLL (SEQ ID NO 53) peptide for Peptide condition, mRNA-eGFP electroporation for mRNA-LFL condition), error bars indicate standard error. Significance tested by Friedman test comparing against control conditions.

FIG. 3: Peptide stabilization of HLA-E surface expression. Assessment of peptide-HLA-E binding via HLA-E surface stabilization assay with RMA-S/HLA-E cells incubated with 30 μM peptide. HLA-E expression is detected with an anti-HLA-E antibody. Measurements from replicate experiments are shown, with solid black lines indicating mean values.

FIG. 4: NK cell stimulation assays. Assessment of effects of peptides on NK cell activity through incubation of NK cells, peptides, and a) K562/HLA-E or b) RMA-S/HLA-E target cells. Peptides were derived from the human or CMV proteomes or are included as positive (LFL, LIL) or negative controls (PQS). Replicates for individual peptides are from different donors, with solid black lines indicating mean values.

FIG. 5: mRNA-LFL prototype induces activation of NKG2C⁺ NK cells in an autologous setting. Activation of NKG2C⁺ NK cells after 6 h of co-culture with autologous monocyte-derived dendritic cells (moDCs) either left untreated or transfected with mRNA-eGFP or mRNA-LFL 16 h before co-culture.

### EXAMPLES

The invention is further described by the following examples. These are not intended to limit the scope of the invention but represent preferred embodiments of aspects of the invention provided for greater illustration of the invention described herein.

### Example 1: Proof of Function for Exemplary Nucleic Acid “mRNA-LFL” VMAPRTLFL

While the administration of synthetic peptides is one potential strategy to translate the inventors previous findings¹⁴ into a therapy, peptide vaccinations have had only limited success in the prevention of infectious diseases or as immunotherapeutic interventions for the treatment of malignant diseases. Potential limitations are poor immunogenicity without adjuvants, antigen-uptake and peptide stability^{23,24}.

In contrast, mRNA immunization approaches have recently been shown to be highly efficient tools for immunization. The inventors initially designed an mRNA vaccine prototype encoding for the HCMV UL40 protein containing the VMAPRTLFL (SEQ ID NO: 2) peptide (“mRNA-UL40”, FIG. 1A). For mRNA stabilization and efficient translation, the inventors placed two copies of the beta-globin 3′UTR in a head-to-tail orientation at the 3′ end of the construct, as previously described²⁶. Surprisingly, this construct did not result in the upregulation of HLA-E on the surface of K562/HLA-E cells, despite efficient expression and delivery of an analogous mRNA encoding for enhanced green fluorescent protein (“mRNA-eGFP”, FIG. 1B). These findings indicated that either the processing of the UL40 protein or the delivery of the peptide into the endoplasmatic reticulum (ER) for loading onto HLA-E was inefficient or required additional factors.

To improve both of these aspects, the inventors designed a new minimal construct for the expression and delivery of the VMAPRTLFL peptide into the ER, to be presented on HLA-E and induce the activation of NKG2C⁺ NK cells (“mRNA-LFL” according to the VMAPRTLFL peptide variant of VMAPRTLXL, FIG. 2A, SEQ ID NO:1). For the efficient presentation of the peptide, a signal sequence targeting the ER is placed in front of the peptide-encoding sequence to shuttle it into the ER, where it is loaded onto HLA-E. To circumvent the requirement for additional processing apart from cleavage of the signal sequence, the inventors decided to only further include the sequence encoding the VMAPRTLFL peptide, without the rest of the UL40 protein.

The mRNA-construct was stabilized by two copies of the beta-globin 3′UTR in a head-to-tail orientation as described above and elsewhere herein. Importantly, the exemplary nucleic acid molecule construct “mRNA-LFL” induced efficient upregulation of HLA-E on K562/HLA-E cells (FIG. 2B). HLA-E stabilization could be observed for at least 24 hours after treatment, indicating clearly prolonged peptide presentation in comparison to the synthetic peptide, which was not detectable after only 6 hours¹⁴ in prior experiments. As a result, co-cultures of primary human NK cells with K562/HLA-E cells transfected with “mRNA-LFL” resulted in efficient and specific activation of NKG2C⁺ NK cells, to a similar level as with high concentrations of the synthetic peptide (FIG. 2C-D). The present mRNA-based delivery therefore enables unexpected improvements over earlier peptide-based approaches. Together, these findings demonstrate efficient expression, processing and presentation of “mRNA-LFL”, supporting its use as a novel mRNA vaccine targeting NKG2C⁺ NK cells.

Finally, to test whether mRNA-LFL can also induce NKG2C⁺ NK cell activation in a more physiological setting with primary, autologous antigen-presenting cells, the inventors established an experimental system in which mRNA-LFL is delivered into monocyte-derived dendritic cells (moDCs) by chemical transfection. Indeed, upon co-culture, moDCs transfected with mRNA-LFL induced activation of NKG2C⁺ NK cells compared to untreated moDCs or moDCs transfected with mRNA-eGFP as negative controls (FIG. 5). These findings demonstrate the ability of mRNA-LFL to engage NKG2C⁺ NK cells when translated and processed by autologous, professional antigen-presenting cells, which underlines its functionality under physiological conditions and therefore further supports its therapeutic potential.

### Example 2: HLA-E Binding and Modulation of NK Cell Activation by Sequence Variants

The inventors investigated which UL-40 derived peptide sequences would be effective in binding HLA-E, upregulation and stabilization of HLA-E, and accordingly achieve activation of NKG2C⁺ NK cells and the corresponding therapeutic effect. To do so, various sequence variants were assessed for their ability to stabilize surface-expressed HLA-E on RMA-S/HLA-E cells, a TAP-deficient mouse tumor cell line engineered to express HLA-E and human β2M (FIG. 3). These experiments support the functionality of sequence variants of such peptides to be expressed from corresponding nucleic acids of the invention.

As shown in FIG. 3, ten human peptides showed strong stabilization of HLA-E (Human peptides INTS1 (aa) 260-268 (RMPPRSVLL; SEQ ID NO:42), HLA-A (aa) 3-11 (VMPPRTLLL; SEQ ID NO: 40), ECEL1 (aa) 269-277 (TLPERTLYL; SEQ ID NO: 36), TACR3 (aa) 226-234 (VMPGRTLCF; SEQ ID NO:41), CREB3L1 (aa) 419-427 (QMPSRSLLF; SEQ ID NO 30), AKAP6 (aa) 388-396 (TLPKRGLFL; SEQ ID NO: 31), MTREX (aa) 490-498 (NMPARTVLF; SEQ ID NO: 44), FBXO41 (aa) 670-678 (ILTDRSLWL: SEQ ID NO:33), SLC52A3 (aa) 354-362 (FLPNRSLLF; SEQ ID NO:37), PISD (aa) 55-63 (TAPARTMFL, SEQ ID NO 43). Of the CMV peptides, UL120 (aa) 72-80, Merlin (VLPHRTQFL; SEQ ID NO:45) showed strong HLA-E binding. Results are consistent with thermal stability measurements taken on a subset of human and CMV proteome-derived predicted peptides. The peptides UBAC₂₂₇₅₋₂₇₉ (SEQ ID NO:35), EMC1 (SEQ ID NO: 38), OR5D14 (SEQ ID NO:39), PQS (SEQ ID NO: 53) as well as the peptide variants UL120_{71-79 AD169} or_{BE/33/2010} (derived from Uniprot UP000008991 and UP000100992), which have an amino acid sequence different from the one of SEQ ID NO: 29 and the peptides according to the invention, however showed no stabilizing effects (FIG. 3 and data not shown), e.g., potentially due to disfavored residues such as, e.g., a Tyrosine in aa position 9 in EMC1 and OR5D14.

The inventors further assessed if peptides, which may be expressed by the nucleic acid molecules according to the invention, possess the ability to modulate NK cell activation, and whether said peptides could affect NK cell activation through interactions with CD94/NKG2A and CD94/NKG2C. These experiments support the functionality of sequence variants of such peptides to be expressed from corresponding nucleic acids of the invention.

Cell-based activation assays were performed with primary NK cells (FIG. 4). Therein, NK cells were co-incubated with K562 peptide-loaded HLA-E expressing cells, as described previously¹⁴. The K562 human MLL cell line is a common tool for the analysis of NK cell cytotoxicity and activation, as said cells facilitate the analysis of both NKG2C-mediated activation and NKG2A-mediated inhibition. The PQS peptide served as a negative control.

Peptides that bound and stabilized HLA-E in the cell-based stability experiment (FIG. 3) were selected and assessed regarding their effect on NKG2A+/NKG2C− or NKG2A−/NKG2C+ NK cell activity. The effect was quantified by the ability to induce NK cell degranulation and IFN-g, TNF and CCL3 production, compared to VMAPRTLIL (“LIL”; SEQ ID NO: 4) and VMAPRTLFL (“LFL”; SEQ ID NO: 2) peptides (class I MHC-derived) as positive control, and VMAPQSLLL (“PQS”; (SEQ ID NO: 53) as negative control peptide (FIG. 4). NKG2A/NKG2C NK cells served as an internal control. NKG2A/NKG2C NK cells have been shown previously to not be affected by peptide-HLA-E complexes.

These experiments revealed that the analyzed peptides according to the invention had both a strong inhibitory effect on NKG2A+ cells and a strong activating effect on NKG2C+ cells, e.g., human peptides INTS1 amin acid (aa) 260-268 (RMPPRSVLL; SEQ ID NO:42), HLA-A aa 3-11 (VMPPRTLLL; SEQ ID NO:40), ECEL1 aa 269-277 (TLPERTLYL; SEQ ID NO: 36), TACR3 aa 226-234 (VMPGRTLCF; SEQ ID NO:41), MTREX aa 490-498 (NMPARTVLF; SEQ ID NO:44); CMV peptide UL120 aa 72-80 (“Merlin”; VLPHRTQFL; SEQ ID NO:45), moderate inhibitory effects on NKG2A⁺ cells while maintaining strong activating effects on NKG2C⁺ cells (Human peptides CREB3L1 aa 419-427 (QMPSRSLLF; SEQ ID NO 30), AKAP6 aa 388-396 (TLPKRGLFL; SEQ ID NO: 31), FBXO41 aa 670-678 (ILTDRSLWL: SEQ ID NO:33), SLC52A3 aa 354-362 (FLPNRSLLF; SEQ ID NO:37). As such, sequence variation of the exemplary VMAPRTLFL peptide is possible without a loss in the inventive properties of the peptide, as shown herein.

### Example 3: Adaptive NKG2C+ NK Cells Differentially Recognize HCMV-Encoded Peptides During Infection

To further ascertain whether distinct UL40-encoded peptides could be differentially recognized by adaptive NKG2C+ NK cells during infection, we inserted the immunomodulatory US2-6 genes into the TB40 BAC4 and genetically modified the UL40 locus of the resulting repaired TB40 (TB40R) HCMV strain to encode either VMAPQSLLL, VMAPRTLIL, or VMAPRTLFL peptides (as described previously, for example in EP3539553B1).

Upon infection of primary human umbilical vein endothelial cells (HUVEC), UL40 transcripts were abundantly present as early as 16 hours post infection, and the genetically modified viruses were equally efficient in infecting HUVEC as well as in modulating HLA class I levels. In line with previous reports, HCMV infection of HUVEC did not result in HLA-E up-regulation. Importantly, adaptive NKG2C+ NK cells displayed significantly elevated TNF, IFN-gamma, CD107 and CCL3 expression in response to HUVEC infected with TB40R^{UL40_VMAPRTLFL} compared to the other strains, while TB40R^{UL40_VMAPRTLIL} elicited slightly increased activation of selected effector functions. Differential activation by HUVEC infected with distinct HCMV strains was further enhanced by IFN-γ treatment, which can prime anti-viral NK-cell functions. In contrast, NKG2C-NK cells were not affected by virus variants even after IFN-α priming.

These findings suggest that single amino acid exchanges within the UL40 protein can be differentially recognized by adaptive NKG2C+ NK cells during infection and imply that selected mutations in UL40 modulate adaptive NKG2C+ NK-cell responses. Nevertheless, sequence variation with the scope of the present invention and the sequences disclosed herein appears to not detrimentally influence the relevant therapeutic effect obtained by the inventive peptides. As outlined in Example 1, the peptide to elicit the relevant biological effect can also be effectively administered and expressed in the form of a nucleic acid encoding said peptide.

### Example 4: Peptide Recognition Controls Relative Accumulation of NKG2C+ NK Cells from HCMV-Individuals in the Presence of Pro-Inflammatory Signals

To examine whether peptide recognition not only affects the differential activation of adaptive NKG2C+ NK cells from HCMV+ individuals but could also influence the extent of NKG2C+ NK-cell proliferation from HCMV-individuals, purified CD56dim NK cells from HCMV-donors were co-cultured with peptide-pulsed target cells in the presence of IL-15 (as described previously, for example in EP3539553B1). Furthermore, sequence variants were tested in this context to support functionality of various UL40 peptides.

In the absence of co-stimulation, only the VMAPRTLFL peptide significantly induced preferential cell division of NKG2C+ NK cells. However, co-engagement of CD2 synergized with peptide recognition and enabled both the VMAPRTLIL and the VMAPRTLFL peptides to drive consistently higher proliferation of NKG2C+ NK cells compared to VMAPQSLLL. Elevated cell division was reflected in increased absolute counts, although not in frequency, of NKG2C+ NK cells after 7 and 14 days of culture. In vivo, CMV generates a systemic inflammatory milieu, which is required for the generation of MCMV-specific adaptive Ly49H+ mouse NK cells. Integration of pro-inflammatory signals by short-term addition of IL-12 and IL-18 (IL-12/18) to VMAPRTLVL-, VMAPRTLIL-, or VMAPRTLFL-pulsed targets resulted in an increase of NKG2C+ NK-cell absolute counts as well as frequencies compared to the non-activating VMAPQSLLL peptide, indicating a permissive role for pro-inflammatory cytokine signals in the accumulation of NKG2C+ cells in a peptide-dependent fashion. In the early culture period, presence of IL-12/18 lead to progressive NK-cell loss independent of peptide recognition, while at later time points, engagement of NKG2C by VMAPRTLFL rescued cell numbers, resulting in increasing frequencies of NKG2C+ NK cells.

In order to gain a quantitative understanding of NKG2C+ NK-cell proliferation and accumulation dynamics in this setting, we modified a Gett-Hodgkin model to explicitly take cell division and cell death as well as non-dividing, dying cells into account. Using experimentally determined precursor frequencies, division times as well as death rates as fitting parameters, the model could describe the data experimentally obtained in the first week of culture, both in the presence and absence of IL-12/18 signaling. Without taking varying cell division times and death rates into account, experimental differences in precursor frequencies alone could not explain the observed dynamics of NKG2C+ NK cells. In the absence of IL-12/18, model analysis revealed shortened division times in the presence of VMAPRTLFL-compared to VMAPQSLLL-pulsed targets, while inferred cell death rates were similar. Provision of pro-inflammatory cytokines during the initial phase of culture resulted in dramatically accelerated NKG2C+ NK-cell division induced by VMAPRTLFL, while death rates were higher in the presence of VMAPQSLLL compared to VMAPRTLFL. Thus, mathematical model analysis suggests that the experimentally observed fast proliferation onset and increased absolute NKG2C+ NK-cell numbers in response to VMAPRTLFL-pulsed targets can be largely explained by accelerated cell division; and presence of IL-12/18 results in slightly decreased cell death upon pulsing with VMAPRTLFL compared to VMAPQSLLL.

Collectively, these data show that combined recognition of distinct HCMV peptides and pro-inflammatory cytokines control the relative accumulation of NKG2C+ NK cells from HCMV-individuals, potentially contributing to the variable size of the adaptive NKG2C+ NK-cell population observed in healthy HCMV+ individuals. Although sequence variation in distinct HCMV peptides leads to some difference in NKG2C+ NK-cell numbers, the various sequences tested all lead to beneficial accumulation of NKG2C+ NK cells in a therapeutic context.

### Example 5: Peptide Recognition and Pro-Inflammatory Cytokines Co-Operate in Guiding the Differentiation of Adaptive NKG2C+ NK Cells

Bac Remodeled receptor repertoires and epigenetic landscapes are hallmarks of adaptive NKG2C+ NK cells in HCMV+ individuals. Since peptide recognition was required to enable relative accumulation of NKG2C+ NK-cells from HCMV-individuals in the presence of IL-12/18, the inventors examined the individual and combined contributions of the potent VMAPRTLFL peptide and of pro-inflammatory cytokines to initiate adaptive NK-cell differentiation (as described previously, for example in EP3539553B1).

While culture with VMAPRTLFL alone only had mild effects, the combination of VMAPRTLFL with IL-12/18 skewed expression of several markers including CD2, Siglec-7, educating KIR, Syk, and CD161 preferentially in NKG2C+ cells. Moreover, short stimulation with IL-12/18 played a predominant role in inducing down-regulation of FcεR1γ and CD7 in both NKG2C+ and NKG2C− cells, while other factors might control NKG2A and DNAM-1 dynamics, since their expression was variable after culture. Notably, levels of DNA methylation of the IFNG conserved non-coding sequence (CNS1), which is hypo-methylated in adaptive NK cells, were strikingly reduced after combined treatment with VMAPRTLFL and IL-12/18. Overall, NKG2C engagement co-operated with pro-inflammatory cytokines in guiding the differentiation of NKG2C+ NK cells from HCMV− individuals. As VMAPRTLFL+IL-12/18 stimulation of NKG2C+ NK cells from HCMV− individuals appeared to most efficiently favor the phenotypic skewing towards adaptive NK cells, we next analyzed the global transcriptional imprinting induced by the combined stimuli. Apart from SIGLEC7, CD7, SYK, and CD2, VMAPRTLFL+IL-12/18 resulted in consistent transcriptional modulation of several other markers associated with adaptive NK cells including NCR3 (NKp30), SH2DB1 (EAT2) and ZBTB32 (PLZP), while the expression of other adaptive genes such as ZBTB16 (PLZF), ZBTB20, ITGAL or CRTAM was altered only in some individuals. Functionally, VMAPRTLFL+IL-12/18 promoted sustained up-regulation of activation and exhaustion markers such as HLA-DR, TNFRSF9 (4-1BB), LAG3, CTLA4, and PDCD1 (PD1) as well as of effector functions including IFNG, TNF, CCL3, CCL4, IL8, CSF2, IL10, GZMB, and TNFSF10 (TRAIL), indicating that the combination of these two stimuli promote broad transcriptional imprinting of phenotypic and effector features typical of adaptive NK cells.

Finally, to substantiate the data obtained from in vitro systems, we monitored a cohort of hematopoietic stem cell transplantation (HSCT) patients, which did or did not reactivate HCMV. Upon detection of HCMV reactivation, the peptide-encoding UL40 region of the strain causing the acute infection was sequenced. Next, the NK-cell phenotype was comparatively analyzed after resolution of acute infection selectively in patients infected with VMAPRTLFL- or VMAPRTLIL-encoding viruses. In line with HCMV causing the expansion of NKG2C+ NK cells, percentages of NKG2C-expressing CD56dim NK cells were elevated in patients with HCMV reactivation, although this was not consistently impacted by HCMV strains and NKG2C+ NK-cell frequencies were dynamic in time among patients infected with both VMAPRTLIL or VMAPRTLFL HCMV variants. Notably, frequencies of NK cells displaying the adaptive phenotype CD2+ Siglec-7-NKG2A-FcεR1γ—were elevated in NKG2C+ NK cells derived from patients infected with VMAPRTLFL-encoding viruses. Conversely, patients infected with VMAPRTLIL variants displayed varying degrees of adaptive NK-cell differentiation. Phenotypic alterations were acquired early after HCMV-reactivation within the NKG2C+ compartment and remained relatively stable over time, implying that this phenomenon is largely uncoupled from NK-cell maturation after HSCT.

Together with the data from in vitro experiments, these findings imply that recognition of distinct UL40-encoded peptides contributes to the accumulation and differentiation of adaptive NKG2C+NK cells during infection. This data provides evidence for sensitive recognition of HCMV-encoded peptides by adaptive NKG2C+ NK cells, which—in co-operation with pro-inflammatory cytokine cues-drives their activation and shapes their population size as well as their phenotypic features in response to HCMV infection.

### Example 6: Induction of a Protective Anti-Tumor Response

The HCMV peptide VMAPRTLFL is also found in the leader sequence of HLA-G, another non-classical MHC class I molecule which is not expressed in most healthy tissues but frequently upregulated by tumors. HLA-G inhibits immune cells by directly binding to inhibitory receptors of the LIR-family, such as LILRB1, which allows tumor cells to evade immune recognition. On the other hand, VMAPRTLFL is cleaved off the signal sequence of HLA-G and presented by HLA-E, making HLA-G/HLA-E co-expressing tumors susceptible to recognition by NKG2C+ NK cells.

As described previously in EP3539553B1, the inventors demonstrated this effect using a cell line transfected with HLA-G. Compared to the untransfected control, HLA-G-expressing cells specifically activated NKG2C+ NK cells, even in the context of low HLA-E expression. The activation was blocked by an antibody against CD94, demonstrating its role in recognition. This data demonstrated the reactivity of NKG2C+ NK cells against HLA G expressing targets. Since in this system HLA-G is extrinsically overexpressed to very high levels and the endogenous expression of HLA-E is low, the inventors assessed activation under blockade of the inhibitory HLA-G receptor LILRB1. The blockade further increased the activation, arguing that the in vivo NK cell response might be even more pronounced, as endogenous expression of HLA-G will not be that much higher than of HLA-E and therefore the inhibitory signal through LILRB1 will be less dominant. Based on these findings, the in vivo expansion of NKG2C+ NK cells, as achieved by the nucleic acid of the invention, represents an immunotherapy to treat HLA-G/HLA-E co-expressing tumors.

### SUMMARY OF THE EXAMPLES

As described herein, the Examples thus demonstrate that the inventive peptide sequences, comprising an amino acid sequence as disclosed herein and expressed from a nucleic acid molecule of the invention, contribute to the accumulation of adaptive NKG2C+ NK cells as a therapeutic intervention against HCMV infection and HLA-G/HLA-E co-expressing tumors.

Additionally, various HCMV-derived peptide sequence variants (optionally in combination with pro-inflammatory cytokines) control the accumulation of NKG2C+ NK cells, also in HCMV-individuals. Furthermore, sequence variation within the scope of the present invention and the sequences disclosed herein appears to not detrimentally influence the relevant therapeutic effect obtained by the inventive peptides. Further examples, technical guidance and supporting evidence for the biological effect of the inventive peptides may be found in EP3539553B1 or U.S. Pat. No. 10,864,245B2, which are incorporated by reference in their entirety.

As outlined in Example 1, the peptide to elicit the relevant biological effect can be effectively expressed from a nucleic acid encoding said peptide. Employing a nucleic acid molecule, for example encoding the peptide sequence X₁₀X₁₁X₁₂X₁₃RX₁₄X₁₅X₁₆X₁₇ (SEQ ID NO:29) or other preferred sequences as disclosed herein, together with an endoplasmic reticulum (ER)-targeting sequence, and preferably a stability inducing motif at its 3′-end, represents effective means to express the peptides, induce upregulation and stabilization of HLA-E, and accordingly achieve activation of NKG2C⁺ NK cells and the corresponding therapeutic effect.

### REFERENCES

1. André, P. et al. Anti-NKG2A mAb Is a Checkpoint Inhibitor that Promotes Anti-tumor Immunity by Unleashing Both T and NK Cells. Cell 175, 1731-1743.e13 (2018).
2. Bukowski, J. F., Woda, B. A., Habu, S., Okumura, K. & Welsh, R. M. Natural killer cell depletion enhances virus synthesis and virus-induced hepatitis in vivo. J Immunol 131, 1531-1538 (1983).
3. Daniels, K. A. et al. Murine cytomegalovirus is regulated by a discrete subset of natural killer cells reactive with monoclonal antibody to Ly49H. J Exp Med 194, 29-44 (2001).
4. Biron, C. A., Byron, K. S. & Sullivan, J. L. Severe herpesvirus infections in an adolescent without natural killer cells. N Engl J Med 320, 1731-1735 (1989).
5. Arase, H., Mocarski, E. S., Campbell, A. E., Hill, A. B. & Lanier, L. L. Direct Recognition of Cytomegalovirus by Activating and Inhibitory NK Cell Receptors. Science 296, 1323-1326 (2002).
6. Dokun, A. O. et al. Specific and nonspecific NK cell activation during virus infection. Nat Immunol 2, 951-956 (2001).
7. Smith, H. R. C. et al. Recognition of a virus-encoded ligand by a natural killer cell activation receptor. Proceedings of the National Academy of Sciences 99, 8826-8831 (2002).
8. Guma, M. et al. Imprint of human cytomegalovirus infection on the NK cell receptor repertoire. Blood 104, 3664-3671 (2004).
9. Schlums, H. et al. Cytomegalovirus infection drives adaptive epigenetic diversification of NK cells with altered signaling and effector function. Immunity 42, 443-456 (2015).
10. Luetke-Eversloh, M. et al. Human cytomegalovirus drives epigenetic imprinting of the IFNG locus in NKG2Chi natural killer cells. PLOS Pathog 10, e1004441 (2014).
11. Lee, N. et al. HLA-E is a major ligand for the natural killer inhibitory receptor CD94/NKG2A. Proc Natl Acad Sci USA 95, 5199-5204 (1998).
12. Valés-Gómez, M., Reyburn, H. T., Erskine, R. A., López-Botet, M. & Strominger, J. L. Kinetics and peptide dependency of the binding of the inhibitory NK receptor CD94/NKG2-A and the activating receptor CD94/NKG2-C to HLA-E. EMBO J 18, 4250-4260 (1999).
13. Heatley, S. L. et al. Polymorphism in Human Cytomegalovirus UL40 Impacts on Recognition of Human Leukocyte Antigen-E (HLA-E) by Natural Killer Cells. J Biol Chem 288, 8679-8690 (2013).
14. Hammer, Q. et al. Peptide-specific recognition of human cytomegalovirus strains controls adaptive natural killer cells. Nat Immunol 19, 453-463 (2018).
15. Cao, K. et al. Donor NKG2C Copy Number: An Independent Predictor for CMV Reactivation After Double Cord Blood Transplantation. Front Immunol 9, 2444 (2018).
16. Yu, X.-X. et al. Donor NKG2C homozygosity contributes to CMV clearance after haploidentical transplantation. JCI Insight 7, e149120 (2022).
17. Vietzen, H., Pollak, K., Honsig, C., Jaksch, P. & Puchhammer-Stöckl, E. NKG2C Deletion Is a Risk Factor for Human Cytomegalovirus Viremia and Disease After Lung Transplantation. J Infect Dis 217, 802-806 (2018).
18. Redondo-Pachón, D. et al. Adaptive NKG2C+ NK Cell Response and the Risk of Cytomegalovirus Infection in Kidney Transplant Recipients. J Immunol 198, 94-101 (2017).
19. Cichocki, F. et al. CD56dimCD57+ NKG2C+ NK cell expansion is associated with reduced leukemia relapse after reduced intensity HCT. Leukemia 30, 456-463 (2016).
20. Romagnani, C., Rückert, T. & Hammer, Q.; EP3539553B1; activation and expansion of NKG2C+ NK cells. at (2021).
21. Romagnani, C., Rückert, T. & Hammer, Q. Activation and expansion of NKG2C+ NK cells US 2019/0314445 A1. at (2019).
22. Capuano, C. et al. Memory NK Cell Features Exploitable in Anticancer Immunotherapy. J Immunol Res 2019, U.S. Pat. No. 8,795,673 (2019).
23. Li, W., Joshi, M. D., Singhania, S., Ramsey, K. H. & Murthy, A. K. Peptide Vaccine: Progress and Challenges. Vaccines (Basel) 2, 515-536 (2014).
24. Di Natale, C., La Manna, S., De Benedictis, I., Brandi, P. & Marasco, D. Perspectives in Peptide-Based Vaccination Strategies for Syndrome Coronavirus 2 Pandemic. Front Pharmacol 11, 578382 (2020).
25. Gejman, R. S. et al. Identification of the Targets of T-cell Receptor Therapeutic Agents and Cells by Use of a High-Throughput Genetic Platform. Cancer Immunol Res 8, 672-684 (2020).
26. Holtkamp, S. et al. Modification of antigen-encoding RNA increases stability, translational efficacy, and T-cell stimulatory capacity of dendritic cells. Blood 108, 4009-4017 (2006).
27. Sahin, U., Holtkamp, S., TÜRECI, Ö. & Kreiter, S. Modifikationen von RNA, die zu einer erhöhten Transkriptstabilität und Translationseffizienz führen. (2012).
28. Russell, J. E. Generation of hyperstable mRNAs. (2009).
29. Gejman, R. & Scheinberg, D. A. Methods and compositions for T-cell epitope screening. (2020).
30. Lanier, L. L. & Hildebrand, W. H. CMV Vaccine and Method of Making and Using the Same. (2020).
31. Vivier, E., Tomasello, E., Baratin, M., Walzer, T. & Ugolini, S. Functions of natural killer cells. Nature immunology 9, 503-510 (2008).
32. Biron, C. A., Byron, K. S. & Sullivan, J. L. Severe herpesvirus infections in an adolescent without natural killer cells. The New England journal of medicine 320, 1731-1735 (1989).
33. O'Sullivan, T. E., Sun, J. C. & Lanier, L. L. Natural Killer Cell Memory. Immunity 43, 634-645 (2015).
34. Jackson, S. E., Mason, G. M. & Wills, M. R. Human cytomegalovirus immunity and immune evasion. Virus Res 157, 151-160 (2011).
35. Braud, V. M. et al. HLA-E binds to natural killer cell receptors CD94/NKG2A, B and C. Nature 391, 795-799 (1998).
36. Lee, N. et al. HLA-E is a major ligand for the natural killer inhibitory receptor CD94/NKG2A. Proceedings of the National Academy of Sciences of the United States of America 95, 5199-5204 (1998).
37. Brooks, A. G. et al. Specific recognition of HLA-E, but not classical, HLA class I molecules by soluble CD94/NKG2A and NK cells. Journal of immunology 162, 305-313 (1999).
38. Guma, M. et al. Expansion of CD94/NKG2C+ NK cells in response to human cytomegalovirus-infected fibroblasts. Blood 107, 3624-3631 (2006).
39. Rolle, A. et al. IL-12-producing monocytes and HLA-E control HCMV-driven NKG2C+ NK cell expansion. The Journal of clinical investigation 124, 5305-5316 (2014).
40. Braud, V., Jones, E. Y. & McMichael, A. The human major histocompatibility complex class Ib molecule HLA-E binds signal sequence-derived peptides with primary anchor residues at positions 2 and 9. European journal of immunology 27, 1164-1169 (1997).
41. Michaelsson, J. et al. A signal peptide derived from hsp60 binds HLA-E and interferes with CD94/NKG2A recognition. The Journal of experimental medicine 196, 1403-1414 (2002).
42. Ulbrecht, M. et al. Cutting edge: the human cytomegalovirus UL40 gene product contains a ligand for HLA-E and prevents NK cell-mediated lysis. Journal of immunology 164, 5019-5022 (2000).
43. Tomasec, P. et al. Surface expression of HLA-E, an inhibitor of natural killer cells, enhanced by human cytomegalovirus gpUL40. Science 287, 1031 (2000).
44. Cerboni, C. et al. Synergistic effect of IFN-gamma and human cytomegalovirus protein UL40 in the HLA-E-dependent protection from NK cell-mediated cytotoxicity. European journal of immunology 31, 2926-2935 (2001).
45. Wang, E. C. et al. UL40-mediated NK evasion during productive infection with human cytomegalovirus. Proceedings of the National Academy of Sciences of the United States of America 99, 7570-7575 (2002).
46. Beziat, V. et al. CMV drives clonal expansion of NKG2C+ NK cells expressing self-specific KIRs in chronic hepatitis patients. European journal of immunology 42, 447-457 (2012).
47. Vivier, E., Tomasello, E., Baratin, M., Walzer, T. & Ugolini, S. Functions of natural killer cells. Nature immunology 9, 503-510 (2008).
48. Biron, C. A., Byron, K. S. & Sullivan, J. L. Severe herpesvirus infections in an adolescent without natural killer cells. The New England journal of medicine 320, 1731-1735 (1989).
49. O'Sullivan, T. E., Sun, J. C. & Lanier, L. L. Natural Killer Cell Memory. Immunity 43, 634-645 (2015).
50. Jackson, S. E., Mason, G. M. & Wills, M. R. Human cytomegalovirus immunity and immune evasion. Virus Res 157, 151-160 (2011).
51. Braud, V. M. et al. HLA-E binds to natural killer cell receptors CD94/NKG2A, B and C. Nature 391, 795-799 (1998).
52. Lee, N. et al. HLA-E is a major ligand for the natural killer inhibitory receptor CD94/NKG2A. Proceedings of the National Academy of Sciences of the United States of America 95, 5199-5204 (1998).
53. Brooks, A. G. et al. Specific recognition of HLA-E, but not classical, HLA class I molecules by soluble CD94/NKG2A and NK cells. Journal of immunology 162, 305-313 (1999).
54. Beaulieu, A. M., Zawislak, C. L., Nakayama, T. & Sun, J. C. The transcription factor Zbtb32 controls the proliferative burst of virus-specific natural killer cells responding to infection. Nature immunology 15, 546-553 (2014).
55. Sun, J. C., Beilke, J. N. & Lanier, L. L. Adaptive immune features of natural killer cells. Nature 457, 557-561 (2009). Rolle, A. et al. IL-12-producing monocytes and HLA-E control HCMV-driven NKG2C+ NK cell expansion. The Journal of clinical investigation 124, 5305-5316 (2014).
56. Braud, V., Jones, E. Y. & McMichael, A. The human major histocompatibility complex class Ib molecule HLA-E binds signal sequence-derived peptides with primary anchor residues at positions 2 and 9. European journal of immunology 27, 1164-1169 (1997).
57. Michaelsson, J. et al. A signal peptide derived from hsp60 binds HLA-E and interferes with CD94/NKG2A recognition. The Journal of experimental medicine 196, 1403-1414 (2002).
58. Ulbrecht, M. et al. Cutting edge: the human cytomegalovirus UL40 gene product contains a ligand for HLA-E and prevents NK cell-mediated lysis. Journal of immunology 164, 5019-5022 (2000).
59. Tomasec, P. et al. Surface expression of HLA-E, an inhibitor of natural killer cells, enhanced by human cytomegalovirus gpUL40. Science 287, 1031 (2000).
60. Cerboni, C. et al. Synergistic effect of IFN-gamma and human cytomegalovirus protein UL40 in the HLA-E-dependent protection from NK cell-mediated cytotoxicity. European journal of immunology 31, 2926-2935 (2001).
61. Wang, E. C. et al. UL40-mediated NK evasion during productive infection with human cytomegalovirus. Proceedings of the National Academy of Sciences of the United States of America 99, 7570-7575 (2002).
62. Pietra G, Romagnani C, et al., “HLA-E-restricted recognition of cytomegalovirus-derived peptides by human CD8+ cytolytic T lymphocytes”. Proc Natl Acad Sci USA. 2003 Sep. 16; 100(19):10896-901.
63. Romagnani C, Pietra G, Falco M, Millo E, Mazzarino P, Biassoni R, Moretta A, Moretta L, Mingari M C. Identification of HLA-E-specific alloreactive T lymphocytes: a cell subset that undergoes preferential expansion in mixed lymphocyte culture and displays a broad cytolytic activity against allogeneic cells. Proc Natl Acad Sci USA. 2002 Aug. 20; 99(17):11328-33. doi: 10.1073/pnas.172369799. Epub 2002 Aug. 7. PMID: 12167676; PMCID: PMC123256.
64. He, W, Gea-Mallorqui, E, Colin-York, H, Fritzsch, M, Gillespie, G M, Brackenridge, S, Borrow, P. Intracellular trafficking of HLA-E and its regulation; JEM. 2023 May 4.
65. Sher Y P, Lin S I, Chai K M, Chen I H, Liu S J. Endoplasmic reticulum-targeting sequence enhanced the cellular immunity of a tumor-associated antigen L6-based DNA vaccine. Am J Cancer Res. 2019 Sep. 1; 9(9):2028-2036. PMID: 31598403; PMCID: PMC6780668.
66. Stevens, J., Joly, E., Trowsdale, J. et al. Peptide binding characteristics of the non-classical class Ib MHC molecule HLA-E assessed by a recombinant random peptide approach. BMC Immunol 2, 5 (2001). https://doi.org/10.1186/1471-2172-2-5.
67. Joseph D. Miller, Dominique A. Weber, Chris Ibegbu, Jan Pohl, John D. Altman, Peter E. Jensen; Analysis of HLA-E Peptide-Binding Specificity and Contact Residues in Bound Peptide Required for Recognition by CD94/NKG2 1. J Immunol 1 Aug. 2003; 171 (3): 1369-1375. https://doi.org/10.4049/jimmunol.171.3.1369.
68. Teufel F, Almagro Armenteros J J, Johansen A R, Gíslason M H, Pihl S I, Tsirigos K D, Winther O, Brunak S, von Heijne G, Nielsen H. SignalP 6.0 predicts all five types of signal peptides using protein language models. Nat Biotechnol. 2022 July; 40(7):1023-1025. doi: 10.1038/s41587-021-01156-3. Epub 2022 Jan. 3. PMID: 34980915; PMCID: PMC9287161.

1. A nucleic acid molecule, comprising:
a. a nucleic acid sequence encoding a peptide sequence X₁₀X₁₁X₁₂X₁₃RX₁₄X₁₅X₁₆X₁₇ (SEQ ID NO:29), wherein
X₁₀ is Threonine, Phenylalanine, Isoleucine, Asparagine, Arginine, Glutamine or Valine,
X₁₁ is Glycine, Asparagine, Alanine, Leucine or Methionine,
X₁₂ is Threonine or Proline,
X₁₃ is Alanine, Tryptophan, Glycine, Proline, Histidine, Asparagine, Aspartic acid, Glutamic acid, Lysine or Serine, R is Arginine,
X₁₄ is Serine, Glycine or Threonine,
X₁₅ is Leucine, Valine, Glutamine or Methionine,
X₁₆ is Tryptophan, Tyrosine, Alanine, Isoleucine, Leucine, Phenylalanine, Valine, Proline, Cysteine or Glycine, and
X₁₇ is Leucine, Phenylalanine or Isoleucine, and
b. an endoplasmic reticulum (ER)-targeting sequence.

2. The nucleic acid molecule according to claim 1, comprising additionally a stability inducing motif at its 3′-end.

3. The nucleic acid molecule according to claim 1, wherein the ER-targeting sequence comprises or consists of a nucleic acid sequence of a mouse mammary tumor virus envelope gene.

4. The nucleic acid molecule according to claim 2, wherein the stability inducing motif comprises or consists of at least one copy of a 3′UTR of a beta-globin gene.

5. The nucleic acid molecule according to claim 1, wherein the nucleic acid molecule is an RNA molecule.

6. The nucleic acid molecule according to claim 1, wherein the nucleic acid sequence comprises or consists of a nucleic acid sequence encoding VMAPRTLXL (SEQ ID NO: 1), wherein X is an amino acid with a hydrophobic side chain selected from A, I, L, F, V, P and G.

7. The nucleic acid molecule according to claim 1, wherein the nucleic acid sequence encodes any one of VMAPRTLFL (SEQ ID NO: 2), VMAPRTLAL (SEQ ID NO: 3), VMAPRTLIL (SEQ ID NO: 4), VMAPRTLLL (SEQ ID NO: 5), VMAPRTLVL (SEQ ID NO: 6), VMAPRTLPL (SEQ ID NO: 7), VMAPRTLGL (SEQ ID NO: 8).

8. The nucleic acid molecule according to claim 1, wherein the nucleic acid sequence comprising the ER-targeting sequence and the stability inducing motif comprises or consist of a nucleic acid sequence according to SEQ ID NO 26, 47, 49-52, respectively.

9. The nucleic acid molecule according to claim 1, wherein the nucleic acid molecule comprises a stop codon before its 3′ UTR, namely 3′ (downstream) of the nucleic acid sequence encoding X₁₀X₁₁X₁₂X₁₃RX₁₄X₁₅X₁₆X₁₇ and 5′ (upstream) of the stability inducing motif.

10. The nucleic acid molecule according to claim 1, wherein the nucleic acid molecule comprises a cleavage site 5′ (upstream) of the nucleic acid sequence encoding X₁₀X₁₁X₁₂X₁₃RX₁₄X₁₅X₁₆X₁₇.

11. The nucleic acid molecule according to claim 1, wherein the nucleic acid molecule comprises a 5′-cap structure.

12. The nucleic acid molecule according to claim 1, wherein the nucleic acid molecule comprises one or more spacer sequences between the nucleic acid sequence encoding X₁₀X₁₁X₁₂X₁₃RX₁₄X₁₅X₁₆X₁₇ and the stability inducing motif and/or the ER-targeting sequence.

13. A method of treating and/or reducing the risk of a medical condition associated with pathogenic cells expressing HLA-E, comprising administering a therapeutically effective amount of the nucleic acid molecule according to claim 1 to a patient to expand and/or activate NKG2C+ natural killer (NK) cells in said patient.

14. A method of treating and/or reducing the risk of a medical condition associated with a human cytomegalovirus (HCMV) infection, comprising administering a therapeutically effective amount of an immunogenic composition, comprising the nucleic acid molecule according to claim 1, to a patient.

15. A method of treating cancer comprising administering a therapeutically effective amount of the nucleic acid molecule according to claim 1 to a patient, wherein said cancer expresses HLA-E.

16. A pharmaceutical composition comprising the nucleic acid molecule according to claim 1, and at least one pharmaceutically acceptable carrier.

17. A pharmaceutical composition according to claim 16, wherein the composition is configured for delivery by nanoparticle, lipofection and/or lipo-nanoparticles.

18. The nucleic acid molecule according to claim 10, wherein the cleavage site is an enzyme cleavage site for an ER-associated peptidase.

19. The nucleic acid molecule according to claim 12, wherein the spacer sequence encodes a cleavage site for an ER-associated peptidase.

20. The method according to claim 14, wherein the risk of reactivation of human cytomegalovirus (HCMV) infections is reduced and/or viral titers in an individual infected with HCMV are reduced.
